(19) United States
(12) Patent Application Publication
(10) Pub. No.: US 2023/0000828 A1
(43) Pub. Date: Jan. 5, 2023

(54) IMPROVING RENAL FUNCTION AFTER KIDNEY TRANSPLANTATION

(71) Applicant: Angion Biomedica Corp., Uniondale, NY (US)

(72) Inventors: Tracy J. Mayne, San Francisco, CA (US); Robert Julian Nordyke, Topanga, CA (US); John Francis Neylan, III, Wellesley, MA (US); Jay R. Venkatesan, Park City, UT (US); Itzhak D. Goldberg, Englewood, NJ (US)

(21) Appl. No.: 17/773,091

(22) PCT Filed: Oct. 30, 2020
(86) PCT No.: PCT/US2020/058403

§ 371 Date: Apr. 29, 2022

Related U.S. Application Data
(60) Provisional application No. 63/042,483, filed on Jun. 22, 2020.
(60) Provisional application No. 62/928,530, filed on Oct. 31, 2019.

(51) Int. Cl. A61K 31/4155; A61K 9/00; A61K 47/10; A61K 47/26; A61P 43/00

(52) U.S. Cl. CPC A61K 31/4155; A61K 9/0019; A61K 47/10; A61K 47/26; A61P 43/00

(57) ABSTRACT

Provided herein are methods of improving kidney function after renal transplantation comprising administering (E)-3-[2-(2-thienyl)vinyl]-1H-pyrazole.

### RELATED APPLICATIONS

This application claims priority to U.S. Provisional Patent Appl. No. 62/928,530, filed Oct. 31, 2019, and U.S. Provisional Patent Appl. No. 63/042,483, filed Jun. 22, 2020, the entire contents of each of which are hereby incorporated by reference.

### BACKGROUND

Delayed graft function (DGF) after kidney transplantation is associated with lower graft survival, higher mortality and greater healthcare costs. Patients displaying DGF lack acceptable autonomous kidney function and require renal replacement therapy in the first week after transplant. Most transplant centers report a DGF rate of 20 to 50% (Boom H, Mallat M J, de Fijter J W, Zwinderman A H, Paul L C. Delayed graft function influences renal function but not survival. Transplant Proc. 2001; 33(1-2):1291). Kidneys that manifest DGF have a higher frequency of adverse outcomes, including decreased graft function, decreased graft survival, and result in increased patient mortality. Additionally, the duration of DGF is an important prognostic factor for graft survival, and studies have reported that prolonged DGF (>6 days) has a deleterious effect on graft survival and that DGF is the single most important determinant of 1 year graft survival (Giral-Classe M, Hourmant M, Cantarovich D, et al. Delayed graft function of more than six days strongly decreases long-term survival of transplanted kidneys. Kidney Int. 1998; 54(3):972-978). Though there are currently no approved therapies for treating DGF, current strategies for managing DGF include supporting patients with dialysis and monitoring for rejection with serial biopsies, typically while patients continue to be administered drugs such as calcineurin inhibitors (CNI), corticosteroids, and mycophenolate mofetil (MMF; an inosine monophosphate dehydrogenase inhibitor). As a result, DGF increases transplant-associated costs because of longer hospital stays, more frequent out-patient clinic visits, increased imaging, increased invasive procedures including inpatient and outpatient dialysis, and pharmacologic therapies. Moreover, if a patient experiences graft failure (e.g., related to DGF), the patient must begin dialysis again, restarting the adverse health and economic cycles.

### SUMMARY

The present disclosure provides certain technologies for improved treatment of delayed graft function (DGF).

In some embodiments, the present disclosure provides methods of treating (e.g., lessening the severity of, such as by delaying onset and/or reducing degree and/or frequency of one or more features of) DGF, which methods may comprise, for example administering a small molecule mimetic of hepatocyte growth factor (HGF, also known as scatter factor (SF)). HGF/SF is a pleiotropic growth factor that stimulates cell growth, cell motility, morphogenesis, and angiogenesis. Certain small molecule mimetics of HGF/SF have been shown to be useful for treating or lessening severity of a variety of diseases, disorders, and conditions.

The present disclosure provides methods of improving renal function in a subject or a population of subjects who have undergone renal transplantation and/or are at risk of DGF. The present disclosure encompasses the recognition that particular modes of administering an HGF/SF mimetic achieve certain desirable outcomes across a population of subjects at risk of DGF. For example, the present disclosure demonstrates that administration of an HGF/SF mimic to a relevant population according to particular regimen(s) can achieve certain treatment effects across that population. In some embodiments, such population may be or comprise subjects who have undergone renal transplantation with a cadaveric kidney (e.g., a kidney from a donor after cardiac death or a kidney from a donor after brain death).

In particular, among other things, the present disclosure provides an insight that provided methods can achieve greater long-term success for kidney transplants (e.g., after 6 months or 12 months) in subjects receiving an HGF/SF mimetic. Thus, the present disclosure encompasses the recognition that administration of HGF/SF mimetics shortly after renal transplantation (e.g., within about 36 hours or within about 30 hours) can provide significant long-term benefits for the health and quality of life for subjects receiving provided therapies.

The present disclosure also provides the insight that provided methods demonstrate durability of particular effects (e.g., of better renal function after transplantation) in subjects receiving an HGF/SF mimetic. For example, in some embodiments, increased renal function, as measured by, e.g., increased estimated glomerular filtration rate and/or decreased serum creatinine concentration, is maintained for at least 28 days, at least 6 months, or at least 12 months after renal transplantation. Without wishing to be bound by any particular theory, subjects with an estimated glomerular filtration rate after at least 28 days, at least 6 months, or at least 12 months that achieves a lower chronic kidney disease (CKD) stage on the National Kidney Foundation's predictive CKD scale are expected to have increased life expectancies, compared to those with a higher CKD stage.

The present disclosure also provides insight that, in some embodiments, Compound 1 may be administered to a subject or population of subjects in need thereof, regardless of the subject's malignancy status (e.g., current malignancy status and/or history of malignancy).

### BRIEF DESCRIPTION OF THE DRAWINGS

FIG. 1 shows the disposition of subjects in the human clinical trial of Example 1.

FIG. 2 shows Kaplan-Meier curves for time to production of ≥1200 cc urine over 24 h by study arm.

FIG. 3 shows mean total daily urine output (in cc) from post-transplant Day 1 to Day 14 by study arm.

FIG. 4 shows least squares mean change from baseline (Day 1) in total daily urine output (in cc) from post-transplant Day 2 to Day 14 by study arm.

FIG. 5 shows least squares mean serum creatinine (mg/dL) over time by study arm.

FIG. 6 shows mean 24-hour urine creatinine clearance (mL/min/1.73 m²) over time by study arm.

FIG. 7 shows serum c-reactive protein (CRP) (mg/dL) by study arm at Day 1 and Day 3 post-transplant.

FIG. 8 shows mean neutrophil gelatinase-associated lipocalin (NGAL) (ng/mL) by study arm at Day 1 and Day 3 post-transplant.

FIG. 9 shows the time to first dialysis session in the first 7 days post-transplant by study arm.

FIG. 10 shows the cumulative number of dialysis sessions through Day 28 by treatment group.

FIG. 11 shows the length of transplant hospitalization (in days) per subject by treatment arm.

FIG. 12 shows least squares mean estimated glomerular filtration rate (eGFR) (mL/min/1.73 m²) by study arm at screening, Day 3, Day 7, Day 14, Day 28, Month 6 and Month 12.

FIG. 13 shows the duration of dialysis through Day 28 by treatment arm.

FIG. 14 shows incidence of graft failure over time by study arm.

FIG. 15 shows mean eGFR (mL/min/1.73 m²) by study arm at screening, Day 3, Day 7, Day 14, Day 28, Month 6 and Month 12, overlaid with the National Kidney Foundation's predictive CKD stages.

FIG. 16A shows percent survival of Compound 1 treatment group vs vehicle treatment group in a first study of an orthotopic glioma model.

FIG. 16B shows percent survival of Compound 1 treatment group vs vehicle treatment group in a second study of an orthotopic glioma model.

FIG. 17A shows colon tumor size of Compound 1 treatment group vs vehicle treatment group in a human colon tumor xenograft model.

FIG. 17B shows colon tumor weight of Compound 1 treatment group vs vehicle treatment group in a human colon tumor xenograft model. (ns=not significant).

FIG. 18A shows pancreatic tumor volume of Compound 1 treatment group vs vehicle treatment group in a human pancreatic tumor xenograft model.

FIG. 18B shows pancreatic tumor weight of Compound 1 treatment group vs vehicle treatment group in a human pancreatic tumor xenograft model.

FIG. 19A shows the lowest two quintiles (solid lines) of patients with worst urine output within 24 hours after a kidney transplantation have a significantly decreased chance of their new kidney surviving for five years compared to the highest quintile (dashed line) (Schnuelle, P. et al. Nephrol. Dial. Transplant (2007) 22:235-45).

FIG. 19B shows patients with the highest eGFR (small dotted line) at discharge after a kidney transplantation have better cumulative kidney transplant survival as compared with patients with lower eGFR at discharge (large dotted and solid lines) (based on data reported in Schnuelle, P. et al. Nephrol. Dial. Transplant (2007) 22:235-45).

FIG. 20 provides XRPD pattern of Compound 1 Lot I.

FIG. 21 provides TGA curve of Compound 1 Lot I.

FIG. 22 provides DSC thermogram of Compound 1 Lot I.

FIG. 23 provides single crystal X-ray crystallography of Compound 1 Form A. N and S atoms are labeled; unlabeled non-hydrogen atoms are carbon.

FIG. 24 provides XRPD pattern of Compound 1 Form A calculated from single crystal X-ray diffraction data.

FIG. 25 provides XRPD pattern of Compound 1 Form A.

FIG. 26 provides TGA curve of Compound 1 Form A.

FIG. 27 provides DSC thermogram of Compound 1 Form A.

FIG. 28 provides a comparison of XRPD patterns of Compound 1 Lot I and Compound 1 Single Crystal Form A.

FIG. 29A shows BUN levels in rats after 24 hours of reperfusion. Data are presented as mean±SEM (male, n=16 and female, n=10).

FIG. 29B shows SCr levels in rats after 24 hours of reperfusion. Data are presented as mean±SEM (male, n=16 and female, n=10).

FIG. 30A shows BUN levels in rats treated QD with Compound 1 vs. vehicle initiated 24 hours post-reperfusion. Data are presented as mean±SEM. Normal rats are untreated rats that were not subjected to renal ischemia-reperfusion.

FIG. 30B shows SCr levels in rats treated QD with Compound 1 vs. vehicle initiated 24 hours post-reperfusion. Data are presented as mean±SEM. Normal rats are untreated rats that were not subjected to renal ischemia-reperfusion.

FIG. 30C shows urine output in rats treated QD with Compound 1 vs. vehicle initiated 24 hours post-reperfusion. Data are presented as mean±SEM. Normal rats are untreated rats that were not subjected to renal ischemia-reperfusion.

FIG. 30D shows survival of rats treated QD with Compound 1 vs. vehicle initiated 24 hours post-reperfusion.

FIG. 31A shows BUN levels in dogs treated QD with Compound 1 vs. vehicle initiated at onset of reperfusion or 1 day post-reperfusion.

FIG. 31B shows SCr levels in dogs treated QD with Compound 1 vs. vehicle initiated at onset of reperfusion or 1 day post-reperfusion.

### DETAILED DESCRIPTION

### Definitions

The term “about”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the term “administering” or “administration” typically refers to the administration of a composition to a subject to achieve delivery of an active agent to a site of interest (e.g., a target site which may, in some embodiments, be a site of disease or damage, and/or a site of responsive processes, cells, tissues, etc.) As will be understood by those skilled in the art, reading the present disclosure, in some embodiments, one or more particular routes of administration may be feasible and/or useful in the practice of the present disclosure. For example, in some embodiments, administration may be parenteral. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. As described herein, in many embodiments, administration is parenteral, e.g., via intravenous (IV) administration, which in some embodiments may be or comprise IV perfusion); in some embodiments, one or more instances of perfusion may be performed. In some embodiments, amount perfused and/or rate of perfusion may be selected, for example, in light of a characteristic such as subject weight, age, presence and/or extent of one or more relevant symptom(s), timing relative to transplant procedure, etc.

As used herein, the term “comparable” refers to two or more agents, entities, situations, sets of conditions, circumstances, individuals, or populations, etc., that may not be identical to one another but that are sufficiently similar to permit comparison there between so that one skilled in the art will appreciate that conclusions may reasonably be drawn based on differences or similarities observed. In some embodiments, comparable agents, entities, situations, sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, circumstances, individuals, or populations, etc. to be considered comparable. For example, those of ordinary skill in the art will appreciate that sets of circumstances, agents, entities, situations, individuals, or populations are comparable to one another when characterized by a sufficient number and type of substantially identical features to warrant a reasonable conclusion that differences in results obtained or phenomena observed under or with different agents, entities, situations, sets of circumstances, individuals, or populations are caused by or indicative of the variation in those features that are varied.

As used herein, the term “pharmaceutical composition” refers to a composition comprising a pharmaceutical active (which may be, comprise, or otherwise become an active agent upon administration of the composition), formulated together with one or more pharmaceutically acceptable carriers. In some embodiments, a pharmaceutical composition is or comprises a pharmaceutical active present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, as described herein, a pharmaceutical composition is formulated for parenteral administration (e.g., for IV administration such as by infusion).

The term “pharmaceutically acceptable salt form,” as used herein, refers to a form of a relevant compound as a salt appropriate for use in pharmaceutical contexts, i.e., salts which are, within the scope of sound medical judgment, suitable for use in contact with the tissues of humans and/or lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

As used herein, the term “reference” describes a standard or control relative to which a comparison is performed. For example, in some embodiments, an agent, individual, population, sample, sequence or value of interest is compared with a reference or control agent, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

As will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

As used herein, the term “subject” refers an organism, typically a mammal (e.g., a human). In some embodiments, a subject is suffering from a relevant disease, disorder or condition. In some embodiments, a human subject is an adult, adolescent, or pediatric subject. In some embodiments, a subject is at risk of (e.g., susceptible to), e.g., at elevated risk of relative to an appropriate control individual or population thereof, a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is an individual to whom diagnosis and/or therapy and/or prophylaxis is and/or has been administered. The terms “subject” and “patient” are used interchangeably herein.

### Hepatocyte Growth Factor Mimetics

PCT Application No. PCT/US2003/040917, filed Dec. 19, 2003 and published as WO2004/058721 on Jul. 15, 2004, the entirety of which is hereby incorporated by reference, describes certain compounds that act as HGF/SF mimetics. Such compounds include Compound 1:

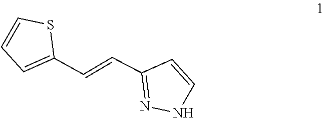 [SMILES: C(=C\c1cccs1)/c1cc[nH]n1]

or a pharmaceutically acceptable salt thereof (i.e., Compound 1 in a pharmaceutically acceptable salt form). Compound 1 has been demonstrated to be remarkably useful for treatment of a variety of conditions including, for example, fibrotic liver disease, ischemia-reperfusion injury, cerebral infarction, ischemic heart disease, renal disease, lung fibrosis, damaged and/or ischemic organs, transplants or grafts, stroke, cerebrovascular disease, and renal fibrosis, among others (see, for example, WO 2004/058721, WO 2010/005580, US 2011/0230407, U.S. Pat. No. 7,879,898, and WO 2009/064422, each of which is hereby incorporated by reference.) In particular, Compound 1 is or has been the subject of clinical trials for delayed graft function in recipients of a deceased donor kidney (Clinicaltrials.gov identifier: NCT02474667), as well as acute kidney injury after cardiac surgery involving cardiopulmonary bypass (Clinicaltrials.gov identifier: NCT02771509), and COVID-19 pneumonia (Clinicaltrials.gov identifier: NCT04459676). Compound 1 is, inter alia, useful in methods provided herein, e.g., for treating DGF and/or improving renal function after kidney transplantation.

Compound 1 has a CAS Registry No. of 1070881-42-3 and is also known by at least the following names:
Terevalefim;
3-[(1E)-2-(thiophen-2-yl)ethen-1-yl]-1H-pyrazole; and
(E)-3-[2-(2-thienyl)vinyl]-1H-pyrazole.

A synthesis of Compound 1, (E)-3-[2-(2-thienyl)vinyl]-1H-pyrazole, is described in detail in Example 7 of WO2004/058721, as well as in Example 2 herein. An alternative synthesis of Compound 1 is provided in Example 6 herein.

Those skilled in the art will appreciate that Compound 1 has a structure that can exist in various tautomeric forms, including (E)-3-[2-(2-thienyl)vinyl]-1H-pyrazole and (E)-5-[2-(2-thienyl)vinyl]-1H-pyrazole, or any mixture thereof. Moreover, those skilled in the art, reading the present disclosure will appreciate that, in many embodiments, teachings described herein are not limited to any particular tautomeric form. Accordingly, in some embodiments, Compound 1 may be referred to as (E)-3(5)-[2-(2-thienyl)vinyl]-1H-pyrazole. The present disclosure contemplates use of all tautomeric forms of Compound 1.

In some embodiments, Compound 1 is provided and/or utilized (e.g., for inclusion in a composition and/or for delivery to a subject) in accordance with the present disclosure in a form such as a salt form. As already noted herein, pharmaceutically acceptable salts are well known in the art.

In some embodiments, Compound 1 is provided and/or utilized (e.g., for inclusion in (e.g., during one or more steps of manufacturing of) a composition and/or for delivery to a subject) in accordance with the present disclosure in a form such as a solid form. Certain solid forms of Compound 1 are described in PCT Application No. PCT/US2020/027710, filed Apr. 10, 2020 and published as WO 2020/210657 on Oct. 15, 2020, the entirety of which is hereby incorporated by reference. In some embodiments, Compound 1 is provided and/or utilized in accordance with the present disclosure in an amorphous solid form, in a crystalline solid form, or in a mixture thereof. In some embodiments, a composition is substantially free of amorphous Compound 1. As used herein, the term “substantially free” means lacking a significant amount (e.g., less than about 10%, less than about 5%, less than about 3%, less than about 2%, or less than about 1%). In some embodiments, a composition comprises at least about 90% by weight of crystalline Compound 1. In some embodiments, a composition comprises at least about 95% by weight of crystalline Compound 1. In some embodiments, a composition comprises at least about 97%, about 98%, or about 99% by weight of crystalline Compound 1. In some embodiments, a crystalline solid form may be or comprise a solvate, hydrate, or an unsolvated form. The use of any and all such forms are contemplated by the present disclosure.

In some embodiments, a crystalline solid form of Compound 1 is Form A. In some embodiments, Form A of Compound 1 is unsolvated (e.g., anhydrous).

In some embodiments, Form A is characterized by one or more peaks in its XRPD pattern selected from those at about 8.64, about 11.04, about 17.34, about 25.06, and about 25.70 degrees 2-theta. In some embodiments, Form A is characterized by two or more peaks in its XRPD pattern selected from those at about 8.64, about 11.04, about 17.34, about 25.06, and about 25.70 degrees 2-theta. In some embodiments, Form A is characterized by three or more peaks in its XRPD pattern selected from those at about 8.64, about 11.04, about 17.34, about 25.06, and about 25.70 degrees 2-theta.

In some embodiments, Form A is characterized by peaks in its XRPD pattern at about 8.64, about 11.04, about 17.34, about 25.06, and about 25.70 degrees 2-theta. In some embodiments, Form A is characterized by peaks in its XRPD pattern at about 8.64, about 11.04, about 17.34, about 25.06, and about 25.70 degrees 2-theta, corresponding to d-spacing of about 10.22, about 8.01, about 5.11, about 3.55, and about 3.46 angstroms.

In some embodiments, Form A is characterized by substantially all of the peaks (degrees 2-theta) in its XRPD pattern, optionally corresponding to d-spacing (angstroms), at about:

| 2θ (°) | d-spacing (Å) |
|---|---|
| 8.64 | 10.22 |
| 11.04 | 8.01 |
| 11.67 | 7.57 |
| 16.06 | 5.51 |
| 17.34 | 5.11 |
| 18.27 | 4.85 |
| 18.69 | 4.74 |
| 19.49 | 4.55 |
| 20.66 | 4.30 |
| 21.09 | 4.21 |
| 21.70 | 4.09 |
| 22.10 | 4.02 |
| 22.76 | 3.90 |
| 23.46 | 3.79 |
| 23.74 | 3.74 |
| 25.06 | 3.55 |
| 25.70 | 3.46 |
| 26.12 | 3.41 |
| 26.32 | 3.38 |
| 27.64 | 3.23 |
| 27.78 | 3.21 |
| 28.31 | 3.15 |
| 28.49 | 3.13 |
| 29.04 | 3.07 |
| 29.95 | 2.98 |
| 31.59 | 2.83 |
| 31.82 | 2.81 |
| 32.25 | 2.77 |
| 33.22 | 2.69 |
| 34.21 | 2.62 |
| 34.42 | 2.60 |
| 35.08 | 2.56 |
| 35.53 | 2.52 |
| 36.33 | 2.47 |
| 36.70 | 2.45 |
| 37.16 | 2.42 |
| 37.65 | 2.39 |
| 39.02 | 2.31 |
| 39.60 | 2.27 |
| 39.81 | 2.26 |

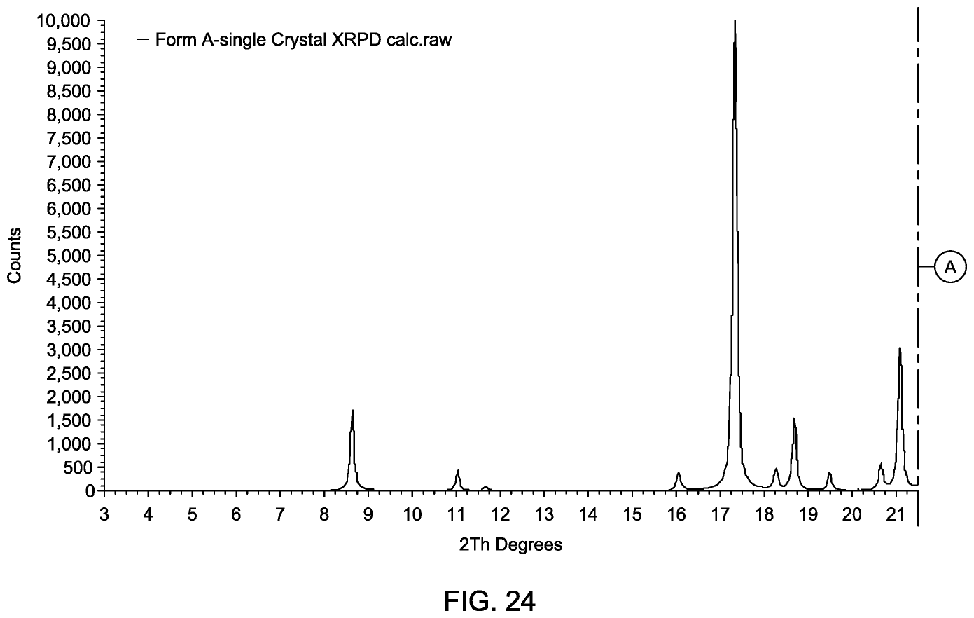

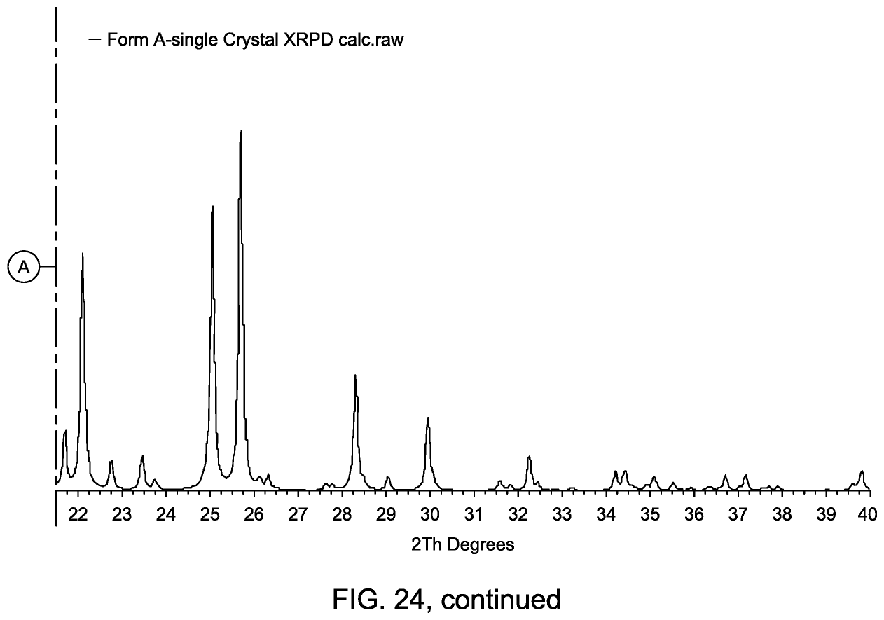

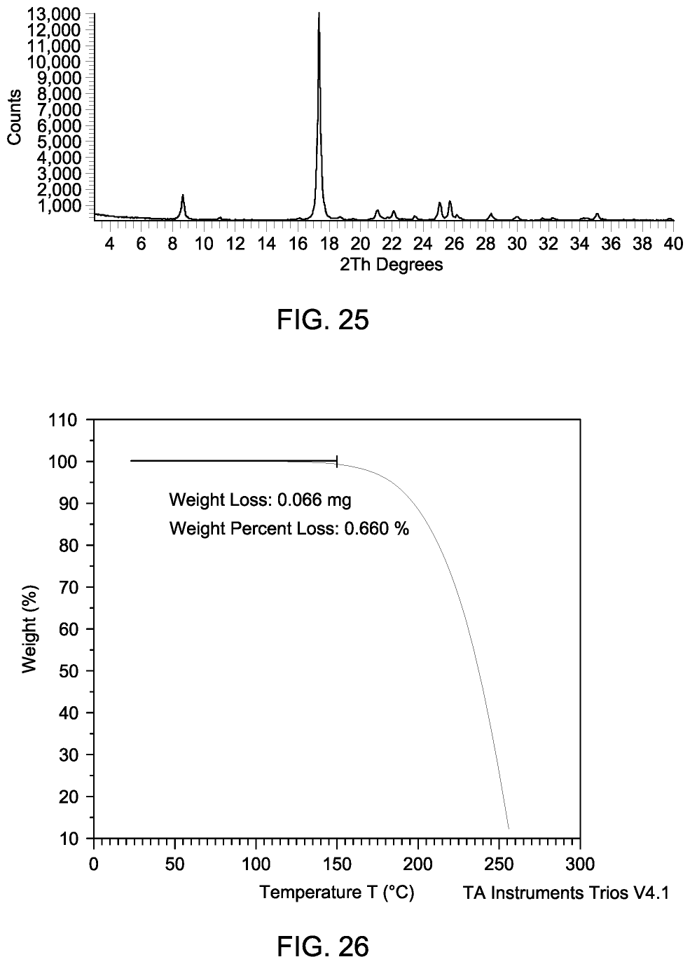

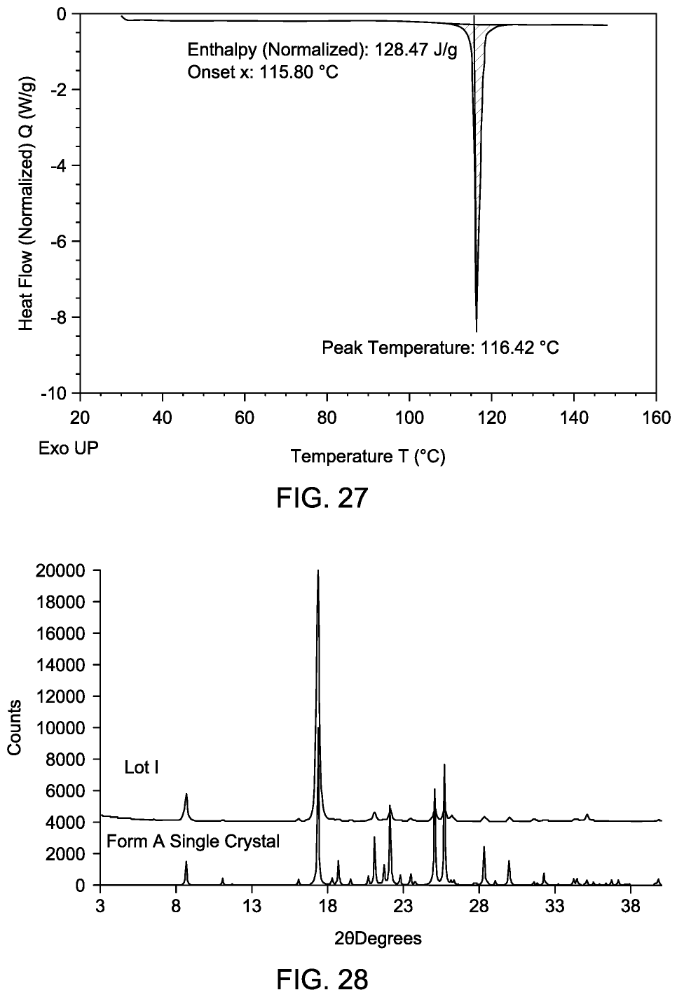

In some embodiments, Form A is characterized by one or more of the following:
(i) an XRPD pattern substantially similar to that depicted in FIG. 24 and/or FIG. 25;
(ii) a TGA pattern substantially similar to that depicted in FIG. 26;
(iii) a DSC pattern substantially similar to that depicted in FIG. 27; and
(iv) a melting point of about 116.42° C.

As used herein, the term “about” when used in reference to a degree 2-theta value refers to the stated value±0.2 degree 2-theta. In some embodiments, “about” refers to the stated value±0.1 degree 2-theta.

Unless otherwise indicated, as used herein “Compound 1” refers to (E)-3-[2-(2-thienyl)vinyl]-1H-pyrazole in any available form, such as, e.g., a tautomer, salt form, and/or solid form thereof.

Certain liquid (e.g., for intravenous or intraperitoneal administration) and solid (e.g., for oral administration) formulations of Compound 1 have been described. See, for example, PCT Application No. PCT/US2009/004014, filed Jul. 9, 2009 and published as WO2010/005580 on Jan. 14, 2010, the entirety of which is hereby incorporated by reference.

In some embodiments, Compound 1 is provided and/or utilized in accordance with the present disclosure as a liquid formulation. In some embodiments, a liquid formulation comprises Compound 1 in a concentration of from about 0.8 mg/mL to about 10 mg/mL.

In some embodiments, a liquid formulation comprising Compound 1 further comprises polyethylene glycol (e.g., polyethylene glycol 300). In some embodiments, a liquid formulation comprises from about 40% (w/v) to about 60% (w/v) polyethylene glycol (e.g., polyethylene glycol 300). In some embodiments, a liquid formulation comprises about 50% (w/v) polyethylene glycol (e.g., polyethylene glycol 300).

In some embodiments, a liquid formulation comprising Compound 1 further comprises polysorbate (e.g., polysorbate 80). In some embodiments, a liquid formulation comprises from about 5% (w/v) to about 15% (w/v) polysorbate (e.g., polysorbate 80). In some embodiments, a liquid formulation comprises about 10% (w/v) polysorbate (e.g., polysorbate 80).

In some embodiments, a liquid formulation comprising Compound 1 is aqueous. In some embodiments, a liquid formulation comprising Compound 1 further comprises saline solution, buffer, or buffered saline solution (e.g., phosphate-buffered saline).

In some embodiments, a liquid formulation comprises Compound 1 and further comprises about 50% (w/v) polyethylene glycol (e.g., polyethylene glycol 300) and about 10% (w/v) polysorbate (e.g., polysorbate 80). In some such embodiments, the liquid formulation is aqueous. In some such embodiments, the liquid formulation further comprises phosphate-buffered saline and/or normal saline.

In some embodiments, Compound 1 is provided and/or utilized in accordance with the present disclosure as a formulation comprising:
about 6 mg/mL to about 10 mg/mL Compound 1;
about 20% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, Compound 1 is provided and/or utilized in accordance with the present disclosure as a formulation comprising:
about 10 mg/mL Compound 1;
about 50% (w/v) polyethylene glycol (e.g., polyethylene glycol 300);
about 10% (w/v) polysorbate (e.g., polysorbate 80); and
one or more aqueous components selected from phosphate buffered saline and normal saline.

In some embodiments, Compound 1 is provided and/or utilized in accordance with the present disclosure as a formulation comprising:
about 10 mg/mL Compound 1;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, Compound 1 is provided and/or utilized in accordance with the present disclosure as a formulation comprising:
about 10 mg/mL Compound 1;
about 50% (w/v) polyethylene glycol (e.g., polyethylene glycol 300);
about 10% (w/v) polysorbate (e.g., polysorbate 80); and about 40% (w/v) phosphate buffered saline.

In some embodiments, Compound 1 is provided and/or utilized in accordance with the present disclosure as a formulation comprising:
about 6 mg/mL Compound 1;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, Compound 1 is provided and/or utilized in accordance with the present disclosure as a formulation comprising:
about 6 mg/mL Compound 1;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

In some embodiments, Compound 1 is provided and/or utilized in accordance with the present disclosure as a formulation comprising:
about 6 mg/mL Compound 1;
about 30% (w/v) polyethylene glycol (e.g., polyethylene glycol 300);
about 6% (w/v) polysorbate (e.g., polysorbate 80);
about 24% (w/v) phosphate buffered saline; and
about 40% (w/v) normal saline.

In some embodiments, liquid formulations of Compound 1 provided herein are prepared by a process comprising a step of combining:
crystalline (E)-3-[2-(2-thienyl)vinyl]-1H-pyrazole (e.g., Form A);
polyethylene glycol (e.g., polyethylene glycol 300);
polysorbate (e.g., polysorbate 80); and
one or more aqueous components (e.g., phosphate buffered saline and/or normal saline) to obtain the formulation.

In some embodiments, a liquid formulation has a pH of about 5 to about 9. In some embodiments, a liquid formulation has a pH of about 6 to about 8. In some embodiments, a liquid formulation has a pH of about 7 (e.g., about 7.4). In some embodiments, a liquid formulation has a pH of about 6.4 to about 8.4 or about 7.4 to about 7.9.

In some embodiments, a liquid formulation is suitable for intravenous administration. In some embodiments, a liquid formulation is suitable for intravenous administration over about 10 min, about 20 min, about 30 min, or about 40 min. In some embodiments, a liquid formulation is suitable for intravenous administration of about 0.5 mg/kg, about 1 mg/kg, about 2 mg/kg, about 3 mg/kg, about 4 mg/kg, about 6 mg/kg, or about 8 mg/kg Compound 1.

### Provided Methods

Provided herein are methods of treating DGF in a subject or in a population of subjects, comprising administering Compound 1 (e.g., by administering a composition that comprises and/or delivers Compound 1 as described herein) to the subject in need thereof. DGF is a severe form of acute kidney injury (AKI) resulting from ischemia-reperfusion injury (caused by oxygen deprivation and reintroduction) following kidney transplantation and defined as the need for dialysis within seven days following transplantation. It is distinct from transplant rejection and is most commonly seen in recipients of deceased-donor kidneys, in part due to the longer periods of warm ischemia (ischemia occurring at body temperature) and cold ischemia (ischemia occurring during kidney preservation and transport) typical for deceased-donor kidney transplants. DGF is most commonly defined as the need for dialysis (i.e., the extracorporeal removal of waste products from the blood when the kidneys are in a state of failure) within seven days following transplantation. In the United States, 70% of the 23,000 kidney transplant procedures performed annually use deceased-donor kidneys, and nearly one-third of these transplant recipients, or more than 5,000 patients per year, are diagnosed with DGF. DGF has a very high clinical and economic burden, and there are no approved therapies.

One of the challenges with DGF stems from the timing of the injury to the kidney, which can occur before the transplantation surgery. For example, in donors who die suddenly (e.g., cardiac death) or who have brain death, the kidney injury occurs when blood flow to the kidney is reduced or stopped, which occurs at or before the time of organ recovery. From that point, the lack of oxygen and nutrients continues to damage the donor kidney until the point at which it is successfully implanted into a recipient, which often takes place between 12 and 24 hours later. After the kidney is transplanted, it undergoes further damage as a result of reperfusion injury caused by the formation of free radicals as oxygen flow returns to the oxygen-starved organ. This combination of damage and its timing makes it difficult for interventions that only block damage pathways, for instance by targeting inflammatory pathways, to work effectively to improve kidney function and reduce the severity of DGF.

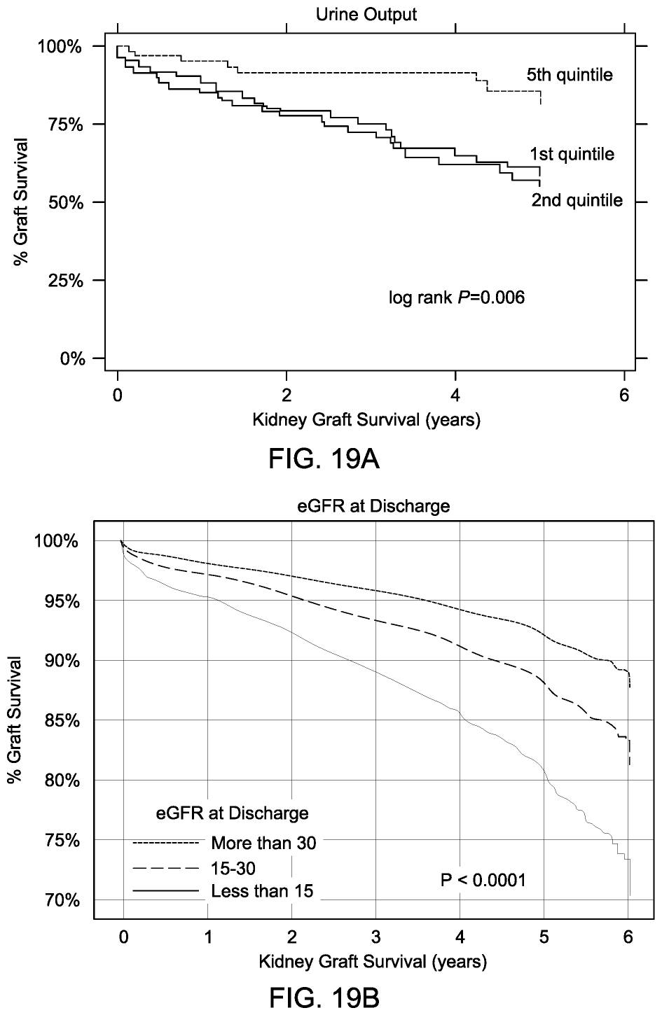

Certain key metrics, including urine output and estimated glomerular filtration rate (eGFR), can be indicative of how a transplanted kidney is performing. Adverse readings in these key metrics can be indicative of kidney damage and more severe forms of DGF, and can be predictive of longer-term negative outcomes, such as reduced survival of the transplanted kidney and increased patient morbidity and mortality (Schnuelle, P. et al. Nephrol. Dial. Transplant (2007) 22:235-45). For example, FIG. 19A shows that the lowest two quintiles of patients (solid lines) with the worst urine output within 24 hours after a kidney transplantation have a significantly decreased chance of their new kidney surviving for five years compared to the highest quintile (dashed line). See Schnuelle, P. et al. 2007. FIG. 19B shows that patients with the highest eGFR at discharge (small dotted line) have better cumulative kidney transplant survival than patients with lower eGFR at discharge (large dotted and solid lines) (based on data reported in Schnuelle, P. et al. 2007). Regardless of the specific measurement used, the occurrence of more severe DGF is associated with an approximate 50% reduction in median graft survival time, from approximately ten years for patients with less severe DGF to approximately five years for patients who experienced more severe DGF. See Schnuelle, P. et al. 2007. Thus, there is a need for therapies for treating (e.g., reducing severity of) DGF.

In some embodiments, the present disclosure provides methods of administering to a subject or population of subjects who have undergone renal transplantation and are at risk of DGF a composition providing Compound 1, according to a regimen established to achieve one or more desirable outcomes. In some embodiments, the regimen is or has been established to achieve one or more desirable outcomes, relative to that observed for a comparable reference population that has not received Compound 1 (e.g., that has received a reference composition which does not deliver Compound 1).

In some embodiments, certain parameters may be evaluated to determine if a desirable outcome is achieved. For example, in some embodiments, certain measures of kidney function, such as serum creatinine (SCr) and/or estimated glomerular filtration rate (eGFR) can be obtained from a subject's blood sample; in some embodiments, such measures can be used to assess if renal function is improving after kidney transplantation. eGFR can be calculated from SCr using, e.g., the MDRD or MDRD4 formula, or the CKD-EPI formula. In some embodiments, eGFR is calculated from SCr using the MDRD or MDRD4 formula. In some embodiments, eGFR is calculated from SCr using the CKD-EPI formula. See Chen, Y.-W., et al. World J. Gastroenterol. 2011 Oct. 28; 17(40):4532-4538. Additionally or alternatively, other indications of kidney function may be evaluated, including urine output, number of dialysis sessions, duration of dialysis, length of hospitalization, and/or incidence of graft failure. Any one or more of parameters such as these may, in some embodiments, be useful for determining short-term and/or long-term efficacy of Compound 1 after renal transplantation.

In some embodiments, the present disclosure provides a method comprising:
administering to a subject or population of subjects who have undergone renal transplantation and are at risk of delayed graft function
a composition providing Compound 1
according to a regimen established to achieve one or more of:
   (i) a greater mean estimated glomerular filtration rate at about 7 days, about 14 days, about 28 days, about 6 months or about 12 months after renal transplantation; (ii) a greater mean increase in estimated glomerular filtration rate from baseline at about 7 days, about 14 days, about 28 days, about 6 months or about 12 months after renal transplantation;
   (iii) a lesser mean serum creatinine concentration at about 7 days, about 14 days, about 28 days, about 6 months or about 12 months after renal transplantation; (iv) a greater mean decrease in serum creatinine concentration from baseline at about 7 days, about 14 days, about 28 days, about 6 months or about 12 months after renal transplantation;
   (v) a greater incidence of achieving 1200 cc urine output over a 24 hour period within 28 days after renal transplantation;
   (vi) a shorter median time to achieve 1200 cc urine output over a 24 hour period within 28 days after renal transplantation;
   (vii) a lesser mean number of dialysis sessions per subject within 28 days after renal transplantation;
   (viii) a shorter mean duration of dialysis within 28 days after renal transplantation;

(ix) a shorter mean length of hospitalization after renal transplantation; and

(x) a lower incidence of graft failure within about 12 months after renal transplantation,

relative to a comparable reference population.

In some embodiments, the present disclosure provides a method comprising:
administering to a subject or population of subjects who have undergone renal transplantation and are at risk of delayed graft function
a composition providing Compound 1
according to a regimen established to achieve one or more of:
   (i) a greater mean estimated glomerular filtration rate at about 6 months or about 12 months after renal transplantation;
   (ii) a greater mean increase in estimated glomerular filtration rate from baseline at about 6 months or about 12 months after renal transplantation;
   (iii) a lesser mean serum creatinine concentration at about 6 months or about 12 months after renal transplantation; and
   (iv) a greater mean decrease in serum creatinine concentration from baseline at about 6 months or about 12 months after renal transplantation,
      relative to a comparable reference population.

In some embodiments, the present disclosure provides a method comprising:
administering to a subject or population of subjects who have undergone renal transplantation and are at risk of delayed graft function
a composition providing Compound 1,
according to a regimen established to achieve one or more of:
   (i) a greater mean estimated glomerular filtration rate at about 12 months after renal transplantation;
   (ii) a greater proportion of patients with an estimated glomerular filtration rate greater than about 30 mL/min/1.73 m² at about 30 days, about 90 days, about 6 months or about 12 months after renal transplantation;
   (iii) a lesser proportion of patients with (1) PNF (defined as a continuous requirement for dialysis for at least 60 days after renal transplantation); (2) DGF (assessed by proportion of patients requiring dialysis within 7 days after renal transplantation); or (3) SGF (defined as having a SCr<3 mg/dL within the first 7 days after renal transplantation without dialysis required);
   (iv) a shorter mean length of hospitalization after renal transplantation; and
   (v) a lesser number of days a patient remains dialysis dependent within the first 30 days after renal transplantation,
      relative to a comparable reference population.

In some embodiments, in methods provided herein, a reference population has not received a composition providing Compound 1. In some embodiments, in methods provided herein, a reference population has received an otherwise comparable reference composition that does not provide Compound 1 (e.g., a placebo, such as normal saline). In some embodiments, in methods provided herein, a reference composition may be or comprise normal saline. In some embodiments, in methods provided herein, a reference composition may be or may have been administered at the same intervals and/or volumes as a composition providing Compound 1.

As used herein, “mean” may refer to an average and/or a least squares mean (LS mean). In some embodiments, “mean” may refer to a LS mean (e.g., a MMRM LS mean). For example, in some embodiments, a regimen has been established to achieve one or both of (i) a greater mean (e.g., a LS mean) estimated glomerular filtration rate at about 6 months or about 12 months after renal transplantation; and (ii) a lesser mean (e.g., a LS mean) serum creatinine concentration at about 6 months or about 12 months after renal transplantation, relative to a comparable reference population.

In some embodiments, a composition providing Compound 1 is administered according to a regimen established to achieve a particular effect, e.g., at a particular time point (e.g., about 7 days, about 14 days, about 28 days, about 6 months and/or about 12 months after renal transplantation). In some embodiments, a composition providing Compound 1 is administered according to a regimen established to achieve a particular effect, e.g., at a particular time point, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise a particular mean estimated glomerular filtration rate (eGFR) at a particular time point. In some embodiments, a particular effect may be or comprise a greater mean estimated glomerular filtration rate (eGFR) at a particular time point, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration); in some embodiments, such time point may be, for example, about 7 days, about 14 days, about 28 days, about 6 months and/or about 12 months after renal transplantation. In some embodiments, a particular effect may be or comprise a greater mean estimated glomerular filtration rate (eGFR) at about 6 months after renal transplantation, relative to an appropriate reference as described herein. In some embodiments, a particular effect may be or comprise a greater mean estimated glomerular filtration rate (eGFR) at about 12 months after renal transplantation, relative to an appropriate reference as described herein. In some embodiments, a particular effect may be or comprise a greater mean estimated glomerular filtration rate (eGFR), calculated using the CKD-EPI equation based on serum creatinine, at about 12 months after renal transplantation, relative to an appropriate reference as described herein.

In some embodiments, the particular effect may be or comprise, for example, a mean eGFR of greater than about 12 mL/min/1.73 m², greater than about 13 mL/min/1.73 m², or greater than about 15 mL/min/1.73 m² at about 7 days after renal transplantation. In some embodiments, the particular effect may be or comprise, for example, a mean eGFR of greater than about 20 mL/min/1.73 m², greater than about 25 mL/min/1.73 m², or greater than about 30 mL/min/1.73 m² at about 14 days after renal transplantation. In some embodiments, the particular effect may be or comprise, for example, a mean eGFR of greater than about 30 mL/min/1.73 m², greater than about 32 mL/min/1.73 m², or greater than about 35 mL/min/1.73 m² at about 28 days after renal transplantation. In some embodiments, the particular effect may be or comprise, for example, a mean eGFR of greater than about 40 mL/min/1.73 m², greater than about 45 mL/min/1.73 m², or greater than about 48 mL/min/1.73 m2 at about 6 months or about 12 months after renal transplantation.

In some embodiments, the particular effect may be or comprise, for example, a mean eGFR of from about 12 mL/min/1.73 m² to about 18 mL/min/1.73 m², from about 13 mL/min/1.73 m² to about 17 mL/min/1.73 m², or from about 14 mL/min/1.73 m² to about 16 mL/min/1.73 m2 at about 7 days after renal transplantation. In some embodiments, the particular effect may be or comprise, for example, a mean eGFR of from about 22 mL/min/1.73 m² to about 36 mL/min/1.73 m², from about 24 mL/min/1.73 m² to about 34 mL/min/1.73 m², or from about 28 mL/min/1.73 m² to about 34 mL/min/1.73 m² at about 14 days after renal transplantation. In some embodiments, the particular effect may be or comprise, for example, a mean eGFR of from about 32 mL/min/1.73 m² to about 44 mL/min/1.73 m², from about 34 mL/min/1.73 m² to about 42 mL/min/1.73 m², or from about 36 mL/min/1.73 m² to about 40 mL/min/1.73 m² at about 28 days after renal transplantation. In some embodiments, the particular effect may be or comprise, for example, a mean eGFR of from about 40 mL/min/1.73 m² to about 55 mL/min/1.73 m², from about 40 mL/min/1.73 m² to about 50 mL/min/1.73 m2, from about 45 mL/min/1.73 m² to about 55 mL/min/1.73 m², from about 45 mL/min/1.73 m² to about 50 mL/min/1.73 m², from about 48 mL/min/1.73 m² to about 55 mL/min/1.73 m², or about 48 mL/min/1.73 m² to about 50 mL/min/1.73 m² at about 6 months or about 12 months after renal transplantation.

In some embodiments, the particular effect may be or comprise, for example, a mean eGFR of about 15.1 mL/min/1.73 m² at about 7 days after renal transplantation. In some embodiments, the particular effect may be or comprise, for example, a mean eGFR of about 32.1 mL/min/1.73 m² at about 14 days after renal transplantation. In some embodiments, the particular effect may be or comprise, for example, a mean eGFR of about 39.0 mL/min/1.73 m² at about 28 days after renal transplantation. In some embodiments, the particular effect may be or comprise, for example, a mean eGFR of about 50.0 mL/min/1.73 m² at about 6 months or about 12 months after renal transplantation. In some embodiments, the particular effect may be or comprise, for example, a mean eGFR of about 50.0 mL/min/1.73 m² at about 6 months after renal transplantation. In some embodiments, the particular effect may be or comprise, for example, a mean eGFR of about 50.1 mL/min/1.73 m² at about 12 months after renal transplantation.

In some embodiments, the particular effect may be or comprise, for example, a mean eGFR that is from about 5 mL/min/1.73 m² to about 15 mL/min/1.73 m², from about 8 mL/min/1.73 m² to about 15 mL/min/1.73 m², from about 8 mL/min/1.73 m² to about 12 mL/min/1.73 m², from about 8 mL/min/1.73 m² to about 13 mL/min/1.73 m², or from about 10 mL/min/1.73 m2 to about 15 mL/min/1.73 m2 greater than that of an appropriate reference as described herein at a particular time point (e.g., at about 7 days, about 14 days, about 28 days, about 6 months or about 12 months after renal transplantation).

In some embodiments, the particular effect may be or comprise, for example, a mean eGFR that is about 5 mL/min/1.73 m², about 8 mL/min/1.73 m², about 10 mL/min/1.73 m2, about 12 mL/min/1.73 m², or about 15 mL/min/1.73 m² greater than that of an appropriate reference as described herein at a particular time point (e.g., at about 14 days, about 28 days, about 6 months or about 12 months after renal transplantation). In some embodiments, the particular effect may be or comprise, for example, a mean eGFR that is about 11.5 mL/min/1.73 m² greater than that of an appropriate reference as described herein at about 14 days after renal transplantation. In some embodiments, the particular effect may be or comprise, for example, a mean eGFR that is about 8.4 mL/min/1.73 m² greater than that of an appropriate reference as described herein at about 28 days after renal transplantation. In some embodiments, the particular effect may be or comprise, for example, a mean eGFR that is about 10.5 mL/min/1.73 m² greater than that of an appropriate reference as described herein at about 6 months after renal transplantation. In some embodiments, the particular effect may be or comprise, for example, a mean eGFR that is about 12.3 mL/min/1.73 m² greater than that of an appropriate reference as described herein at about 12 months after renal transplantation.

In some embodiments, a particular effect may be or comprise achieving a particular CKD stage on the National Kidney Foundation's predictive scale at a particular time point (See, e.g., Example 3). In some embodiments, a particular effect may be or comprise achieving a lower CKD stage on the National Kidney Foundation's predictive scale, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration); in some embodiments, such time point may be, for example, about 7 days, about 14 days, about 28 days, about 6 months and/or about 12 months after renal transplantation.

In some embodiments, the particular effect may be or comprise, for example, a CKD stage on the National Kidney Foundation's predictive scale of 3B or lower, 3A or lower, 2 or lower, or 1 or lower at about 28 days, about 6 months, or about 12 months after renal transplantation. In some embodiments, the particular effect may be or comprise, for example, a CKD stage on the National Kidney Foundation's predictive scale of 3A or lower, 2 or lower, or 1 or lower at about 6 months or about 12 months after renal transplantation.

In some embodiments, a particular effect may be or comprise, for example, a particular mean (e.g., LS mean) increase in eGFR from baseline at a particular time point. In some embodiments, a particular effect may be or comprise a greater mean increase in eGFR from baseline at a particular time point, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration); in some embodiments, such time point may be, for example, about 7 days, about 14 days, about 28 days, about 6 months and/or about 12 months after renal transplantation. In some embodiments, a particular effect may be or comprise a greater mean increase in eGFR from baseline at about 6 months after renal transplantation, relative to an appropriate reference as described herein. In some embodiments, a particular effect may be or comprise a greater mean increase in eGFR from baseline at about 12 months after renal transplantation, relative to an appropriate reference as described herein.

In some embodiments, the particular effect may be or comprise, for example, a mean increase in eGFR from baseline of greater than about 3 mL/min/1.73 m², greater than about 5 mL/min/1.73 m², or greater than about 8 mL/min/1.73 m² at about 7 days after renal transplantation. In some embodiments, the particular effect may be or comprise, for example, a mean increase in eGFR from baseline of greater than about 10 mL/min/1.73 m², greater than about 18 mL/min/1.73 m², or greater than about 22 mL/min/1.73 m² at about 14 days after renal transplantation. In some embodiments, the particular effect may be or comprise, for example, a mean increase in eGFR from baseline of greater than about 20 mL/min/1.73 m², greater than about 25 mL/min/1.73 m², or greater than about 30 mL/min/1.73 m² at about 28 days after renal transplantation. In some embodiments, the particular effect may be or comprise, for example, a mean increase in eGFR from baseline of greater than about 32 mL/min/1.73 m², greater than about 36 mL/min/1.73 m², or greater than about 40 mL/min/1.73 m2 at about 6 months after renal transplantation. In some embodiments, the particular effect may be or comprise, for example, a mean increase in eGFR from baseline of greater than about 27 mL/min/1.73 m², greater than about 36 mL/min/1.73 m2, or greater than about 40 mL/min/1.73 m2 at about 12 months after renal transplantation.

In some embodiments, the particular effect may be or comprise, for example, a mean increase in eGFR from baseline of from about 3 mL/min/1.73 m² to about 10 mL/min/1.73 m2, from about 5 mL/min/1.73 m² to about 10 mL/min/1.73 m², or from about 7 mL/min/1.73 m² to about 9 mL/min/1.73 m² at about 7 days after renal transplantation. In some embodiments, the particular effect may be or comprise, for example, a mean increase in eGFR from baseline of from about 12 mL/min/1.73 m² to about 30 mL/min/1.73 m², from about 15 mL/min/1.73 m² to about 25 mL/min/1.73 m², or from about 20 mL/min/1.73 m² to about 25 mL/min/1.73 m² at about 14 days after renal transplantation. In some embodiments, the particular effect may be or comprise, for example, a mean increase in eGFR from baseline of from about 20 mL/min/1.73 m² to about 35 mL/min/1.73 m², from about 25 mL/min/1.73 m² to about 35 mL/min/1.73 m², or from about 28 mL/min/1.73 m² to about 32 mL/min/1.73 m² at about 28 days after renal transplantation. In some embodiments, the particular effect may be or comprise, for example, a mean increase in eGFR from baseline of from about 32 mL/min/1.73 m² to about 45 mL/min/1.73 m², from about 36 mL/min/1.73 m² to about 45 mL/min/1.73 m², or from about 38 mL/min/1.73 m² to about 42 mL/min/1.73 m² at about 6 months after renal transplantation. In some embodiments, the particular effect may be or comprise, for example, a mean increase in eGFR from baseline of from about 30 mL/min/1.73 m² to about 45 mL/min/1.73 m², from about 36 mL/min/1.73 m² to about 45 mL/min/1.73 m², or from about 39 mL/min/1.73 m² to about 43 mL/min/1.73 m² at about 12 months after renal transplantation.

In some embodiments, a particular effect may be or comprise, for example, a mean increase in eGFR from baseline of about 8.25 mL/min/1.73 m² at about 7 days after renal transplantation. In some embodiments, a particular effect may be or comprise, for example, a mean increase in eGFR from baseline of about 23.36 mL/min/1.73 m² at about 14 days after renal transplantation. In some embodiments, a particular effect may be or comprise, for example, a mean increase in eGFR from baseline of about 30.26 mL/min/1.73 m² at about 28 days after renal transplantation. In some embodiments, a particular effect may be or comprise, for example, a mean increase in eGFR from baseline of about 40.47 mL/min/1.73 m² at about 6 months after renal transplantation. In some embodiments, a particular effect may be or comprise, for example, a mean increase in eGFR from baseline of about 41.23 mL/min/1.73 m² at about 12 months after renal transplantation.

In some embodiments, a particular effect may be or comprise, for example, a mean increase in eGFR from baseline that is from about 5 mL/min/1.73 m² to about 15 mL/min/1.73 m², from about 8 mL/min/1.73 m² to about 15 mL/min/1.73 m², from about 8 mL/min/1.73 m² to about 12 mL/min/1.73 m², from about 8 mL/min/1.73 m² to about 13 mL/min/1.73 m², or from about 10 mL/min/1.73 m² to about 15 mL/min/1.73 m² greater than that than that of an appropriate reference as described herein at a particular time point (e.g., at about 7 days, about 14 days, about 28 days, about 6 months or about 12 months after renal transplantation).

In some embodiments, a particular effect may be or comprise, for example, a mean increase in eGFR from baseline that is about 6 mL/min/1.73 m², about 10 mL/min/1.73 m², about 11 mL/min/1.73 m², about 12 mL/min/1.73 m², or about 13 mL/min/1.73 m² greater than that of an appropriate reference as described herein at a particular time point (e.g., at about 14 days, about 28 days, about 6 months or about 12 months after renal transplantation). In some embodiments, the particular effect may be or comprise, for example, a mean increase in eGFR from baseline that is about 5.79 mL/min/1.73 m² greater than that of an appropriate reference as described herein at about 7 days after renal transplantation. In some embodiments, the particular effect may be or comprise, for example, a mean increase in eGFR from baseline that is about 13.12 mL/min/1.73 m² greater than that of an appropriate reference as described herein at about 14 days after renal transplantation. In some embodiments, the particular effect may be or comprise, for example, a mean increase in eGFR from baseline that is about 10.65 mL/min/1.73 m² greater than that of an appropriate reference as described herein at about 28 days after renal transplantation. In some embodiments, the particular effect may be or comprise, for example, a mean increase in eGFR from baseline that is about 10.08 mL/min/1.73 m² greater than that of an appropriate reference as described herein at about 6 months after renal transplantation. In some embodiments, the particular effect may be or comprise, for example, a mean increase in eGFR from baseline that is about 12.57 mL/min/1.73 m² greater than that of an appropriate reference as described herein at about 12 months after renal transplantation.

In some embodiments, a particular effect may be or comprise, for example, a particular proportion of patients with an eGFR greater than a particular value (e.g., greater than about 30 mL/min/1.73 m²) at a particular time point. In some embodiments, the particular effect may be or comprise, for example, a greater proportion of patients with an eGFR greater than a particular value (e.g., greater than about 30 mL/min/1.73 m²) at a particular time point, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration); in some embodiments, such time point may be, for example, about 30 days, about 90 days, about 6 months and/or about 12 months after renal transplantation.

In some embodiments, a particular effect may be or comprise, for example, a particular mean serum creatinine concentration (SCr) at a particular time point. In some embodiments, the particular effect may be or comprise, for example, a lesser mean SCr at a particular time point, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration); in some embodiments, such time point may be, for example, about 7 days, about 14 days, about 28 days, about 6 months and/or about 12 months after renal transplantation. In some embodiments, the particular effect may be or comprise, for example, a lesser mean SCr at about 6 months after renal transplantation, relative to an appropriate reference as described herein. In some embodiments, the particular effect may be or comprise, for example, a lesser mean SCr at about 12 months after renal transplantation, relative to an appropriate reference as described herein.

In some embodiments, the particular effect may be or comprise, for example, a mean SCr of less than about 6.25 mg/dL, about 6.0 mg/dL, or about 5.90 mg/dL at about 7 days after renal transplantation. In some embodiments, the particular effect may be or comprise, for example, a mean SCr of less than about 4.0 mg/dL, about 3.50 mg/dL, or about 3.30 mg/dL at about 14 days after renal transplantation. In some embodiments, the particular effect may be or comprise, for example, a mean SCr of less than about 2.90 mg/dL, about 2.50 mg/dL, or about 2.40 mg/dL at about 28 days after renal transplantation. In some embodiments, the particular effect may be or comprise, for example, a mean SCr of less than about 1.90 mg/dL, about 1.85 mg/dL, about 1.80 mg/dL, about 1.75 mg/dL, about 1.70 mg/dL, about 1.65 mg/dL, or about 1.60 mg/dL at about 6 months or about 12 months after renal transplantation.

In some embodiments, the particular effect may be or comprise, for example, a mean SCr of from about 5.40 mg/dL to about 6.40 mg/dL, from about 5.50 mg/dL to about 6.0 mg/dL, or from about 5.70 mg/dL to about 5.90 mg/dL at about 7 days after renal transplantation. In some embodiments, the particular effect may be or comprise, for example, a mean SCr of from about 3.0 mg/dL to about 4.0 mg/dL, from about 3.10 mg/dL to about 3.50 mg/dL, or from about 3.20 mg/dL to about 3.30 mg/dL at about 14 days after renal transplantation. In some embodiments, the particular effect may be or comprise, for example, a mean SCr of from about 2.0 mg/dL to about 2.95 mg/dL, from about 2.10 mg/dL to about 2.50 mg/dL, or from about 2.20 mg/dL to about 2.35 mg/dL at about 28 days after renal transplantation. In some embodiments, the particular effect may be or comprise, for example, a mean SCr of from about 1.40 mg/dL to about 1.90 mg/dL, from about 1.55 mg/dL to about 1.85 mg/dL, from about 1.50 mg/dL to about 1.80 mg/dL, from about 1.45 mg/dL to about 1.75 mg/dL, from about 1.40 mg/dL to about 1.65 mg/dL, from about 1.45 mg/dL to about 1.55 mg/dL, or from about 1.55 mg/dL to about 1.65 mg/dL at about 6 months or about 12 months after renal transplantation.

In some embodiments, the particular effect may be or comprise, for example, a mean SCr of about 5.82 mg/dL at about 7 days after renal transplantation. In some embodiments, the particular effect may be or comprise, for example, a mean SCr of about 3.24 mg/dL at about 14 days after renal transplantation. In some embodiments, the particular effect may be or comprise, for example, a mean SCr of about 2.29 mg/dL at about 28 days after renal transplantation. In some embodiments, the particular effect may be or comprise, for example, a mean SCr of about 1.60 mg/dL at about 6 months after renal transplantation. In some embodiments, the particular effect may be or comprise, for example, a mean SCr of about 1.48 mg/dL at about 12 months after renal transplantation.

In some embodiments, the particular effect may be or comprise, for example, a mean SCr that is from about 0.50 mg/dL to about 1.0 mg/dL, from about 0.60 mg/dL to about 0.90 mg/dL, or from about 0.65 mg/dL to about 0.85 mg/dL less than that of an appropriate reference as described herein at a particular time point (e.g., at about 7 days, about 14 days, or about 28 days after renal transplantation). In some embodiments the particular effect may be or comprise, for example, a mean SCr that is from about 0.20 mg/dL to about 0.50 mg/dL, from about 0.25 mg/dL to about 0.45 mg/dL, from about 0.25 mg/dL to about 0.35 mg/dL, from about 0.35 mg/dL to about 0.45 mg/dL, from about 0.28 mg/dL to about 0.32 mg/dL, or from about 0.40 mg/dL to about 0.45 mg/dL less than that of an appropriate reference as described herein at a particular time point (e.g., at about 6 months or about 12 months after renal transplantation).

In some embodiments, the particular effect may be or comprise, for example, a mean SCr that is about 0.90 mg/dL, about 0.85 mg/dL, about 0.80 mg/dL, about 0.75 mg/dL, about 0.70 mg/dL, or about 0.65 mg/dL less than that of an appropriate reference as described herein at a particular time point (e.g., at about 7 days, about 14 days, or about 28 days after renal transplantation). In some embodiments, the particular effect may be or comprise, for example, a mean SCr that is about 0.20 mg/dL, about 0.25 mg/dL, about 0.30 mg/dL, about 0.35 mg/dL, about 0.40 mg/dL, about 0.45 mg/dL, or about 0.50 mg/dL less than that of an appropriate reference as described herein at a particular time point (e.g., at about 6 months or about 12 months after renal transplantation).

In some embodiments, the particular effect may be or comprise, for example, a mean SCr that is about 0.67 mg/dL less than that of an appropriate reference as described herein at about 7 days after renal transplantation. In some embodiments, the particular effect may be or comprise, for example, a mean SCr that is about 0.79 mg/dL less than that of an appropriate reference as described herein at about 14 days after renal transplantation. In some embodiments, the particular effect may be or comprise, for example, a mean SCr that is about 0.70 mg/dL less than that of an appropriate reference as described herein at about 28 days after renal transplantation. In some embodiments, the particular effect may be or comprise, for example, a mean SCr that is about 0.29 mg/dL less than that of an appropriate reference as described herein at about 6 months after renal transplantation. In some embodiments, the particular effect may be or comprise, for example, a mean SCr that is about 0.43 mg/dL less than that of an appropriate reference as described herein at about 12 months after renal transplantation.

In some embodiments, a particular effect may be or comprise, for example, a particular mean decrease in SCr from baseline at a particular time point. In some embodiments, the particular effect may be or comprise, for example, a greater mean decrease in SCr from baseline at a particular time point, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration); in some embodiments, such time point may be, for example, about 7 days, about 14 days, about 28 days, about 6 months and/or about 12 months after renal transplantation. In some embodiments, the particular effect may be or comprise, for example, a greater mean decrease in SCr from baseline at about 6 months after renal transplantation, relative to an appropriate reference as described herein. In some embodiments, the particular effect may be or comprise, for example, a greater mean decrease in SCr from baseline at about 12 months after renal transplantation, relative to an appropriate reference as described herein.

In some embodiments, a particular effect may be or comprise, for example, a mean decrease in SCr from baseline of greater than about 1.5 mg/dL, greater than about 1.8 mg/dL, or greater than about 2.0 mg/dL at about 7 days after renal transplantation. In some embodiments, a particular effect may be or comprise, for example, a mean decrease in SCr from baseline of greater than about 3.9 mg/dL, greater than about 4.2 mg/dL, or greater than about 4.5 mg/dL at about 14 days after renal transplantation. In some embodiments, a particular effect may be or comprise, for example, a mean decrease in SCr from baseline of greater than about 5.0 mg/dL, greater than about 5.3 mg/dL, or greater than about 5.5 mg/dL at about 28 days after renal transplantation. In some embodiments, a particular effect may be or comprise, for example, a mean decrease in SCr from baseline of greater than about 6.0 mg/dL, greater than about 6.1 mg/dL, or greater than about 6.2 mg/dL at about 6 months after renal transplantation. In some embodiments, a particular effect may be or comprise, for example, a mean decrease in SCr from baseline of greater than about 6.0 mg/dL, greater than about 6.2 mg/dL, or greater than about 6.4 mg/dL at about 12 months after renal transplantation.

In some embodiments, a particular effect may be or comprise, for example, a mean decrease in SCr from baseline of from about 1.4 mg/dL to about 2.5 mg/dL, from about 1.6 mg/dL to about 2.2 mg/dL, or from about 1.9 mg/dL to about 2.1 mg/dL at about 7 days after renal transplantation. In some embodiments, a particular effect may be or comprise, for example, a mean decrease in SCr from baseline of from about 3.9 mg/dL to about 5.0 mg/dL, from about 4.4 mg/dL to about 4.9 mg/dL, or from about 4.5 mg/dL to about 4.7 mg/dL at about 14 days after renal transplantation. In some embodiments, a particular effect may be or comprise, for example, a mean decrease in SCr from baseline of from about 4.9 mg/dL to about 6.0 mg/dL, from about 5.4 mg/dL to about 5.8 mg/dL, or from about 5.5 mg/dL to about 5.7 mg/dL at about 28 days after renal transplantation. In some embodiments, a particular effect may be or comprise, for example, a mean decrease in SCr from baseline of from about 6.0 mg/dL to about 6.8 mg/dL, from about 6.1 mg/dL to about 6.5 mg/dL, or from about 6.2 mg/dL to about 6.4 mg/dL at about 6 months after renal transplantation. In some embodiments, a particular effect may be or comprise, for example, a mean decrease in SCr from baseline of from about 6.0 mg/dL to about 6.8 mg/dL, from about 6.2 mg/dL to about 6.6 mg/dL, or from about 6.3 mg/dL to about 6.5 mg/dL at about 12 months after renal transplantation.

In some embodiments, a particular effect may be or comprise, for example, a mean decrease in SCr from baseline that is about 2.05 mg/dL at about 7 days after renal transplantation. In some embodiments, a particular effect may be or comprise, for example, a mean decrease in SCr from baseline that is about 4.63 mg/dL at about 14 days after renal transplantation. In some embodiments, a particular effect may be or comprise, for example, a mean decrease in SCr from baseline that is about 5.59 mg/dL at about 28 days after renal transplantation. In some embodiments, a particular effect may be or comprise, for example, a mean decrease in SCr from baseline that is about 6.27 mg/dL at about 6 months after renal transplantation. In some embodiments, a particular effect may be or comprise, for example, a mean decrease in SCr from baseline that is about 6.40 mg/dL at about 12 months after renal transplantation.

In some embodiments, a particular effect may be or comprise, for example, a mean decrease in SCr from baseline that is from about 0.50 mg/dL to about 1.0 mg/dL, from about 0.60 mg/dL to about 0.90 mg/dL, or from about 0.65 mg/dL to about 0.85 mg/dL greater than that of an appropriate reference as described herein at a particular time point (e.g., at about 7 days, about 14 days, or about 28 days after renal transplantation). In some embodiments the particular effect may be or comprise, for example, a mean SCr that is from about 0.10 mg/dL to about 0.60 mg/dL, from about 0.20 mg/dL to about 0.50 mg/dL, or from about 0.25 mg/dL to about 0.45 mg/dL greater than that of an appropriate reference as described herein at a particular time point (e.g., at about 6 months or about 12 months after renal transplantation).

In some embodiments, a particular effect may be or comprise, for example, a mean decrease in SCr from baseline that is about 0.85 mg/dL, about 0.80 mg/dL, about 0.75 mg/dL, about 0.70 mg/dL, or about 0.65 mg/dL greater than that of an appropriate reference as described herein at a particular time point (e.g., at about 7 days, about 14 days, or about 28 days after renal transplantation). In some embodiments, the particular effect may be or comprise, for example, a mean decrease in SCr from baseline that is about 0.20 mg/dL, about 0.25 mg/dL, about 0.30 mg/dL, about 0.35 mg/dL, about 0.40 mg/dL, about 0.45 mg/dL, or about 0.50 mg/dL greater than that of an appropriate reference as described herein at a particular time point (e.g., at about 6 months or about 12 months after renal transplantation).

In some embodiments, a particular effect may be or comprise, for example, a particular incidence of achieving 1200 cc urine output over a 24 hour period within 28 days after renal transplantation. In some embodiments, the particular effect may be or comprise, for example, a greater incidence of achieving 1200 cc urine output over a 24 hour period within 28 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration). In some embodiments, the particular effect may be or comprise, for example, at least about 50%, at least about 60%, at least about 70%, or at least about 75% of subjects achieving 1200 cc urine output over a 24 hour period within 28 days after renal transplantation. In some embodiments, the particular effect may be or comprise, for example, from about 45% to about 90%, from about 50% to about 85%, from about 60% to about 80% or from about 70% to about 80% of subjects achieving 1200 cc urine output over a 24 hour period within 28 days after renal transplantation. In some embodiments, the particular effect may be or comprise, for example, a percentage of subjects achieving 1200 cc urine output over a 24 hour period within 28 days after renal transplantation that is about 25, about 30, about 35, about 40, or about 45 percentage points greater than that of an appropriate reference as described herein.

In some embodiments, a particular effect may be or comprise, for example, a particular median time for the subjects to achieve 1200 cc urine output over a 24 hour period. In some embodiments, the particular effect may be or comprise, for example, a shorter median time for the subjects to achieve 1200 cc urine output over a 24 hour period, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration). In some embodiments, the particular effect may be or comprise, for example, a median time for the subjects to achieve 1200 cc urine output over a 24 hour period from about 2 days to about 12 days, from about 3 days to about 10 days, or from about 4 days to about 8 days. In some embodiments, the median time for the subjects to achieve 1200 cc urine output over a 24 hour period is about 2 days, about 4 days, about 5 days, about 6 days, or about 8 days. In some embodiments, the particular effect may be or comprise, for example, a median time for the subjects to achieve 1200 cc urine output over a 24 hour period is about 12 days, about 10 days, about 9 days, about 8 days, or about 6 days shorter than that of an appropriate reference as described herein. In some embodiments, the median time for the subjects to achieve 1200 cc urine output over a 24 hour period is about 9 days shorter than that of an appropriate reference as described herein.

In some embodiments, the particular effect may be or comprise, for example, a particular mean number of dialysis sessions per subject within 28 days after renal transplantation. In some embodiments, the particular effect may be or comprise, for example, a lesser mean number of dialysis sessions per subject within 28 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration). In some embodiments, the particular effect may be or comprise, for example, a number of dialysis sessions of less than about 3.8, less than about 3.5, or less than about 3.0 per subject within 28 days after renal transplantation. In some embodiments, the particular effect may be or comprise, for example, a number of dialysis sessions of from about 2.0 to about 3.8, from about 2.5 to about 3.5, or from about 2.7 to about 3.0 per subject within 28 days after renal transplantation. In some embodiments, the particular effect may be or comprise, for example, a number of dialysis sessions of at least about 0.5, at least about 0.75 or at least about 1.0 fewer per subject within 28 days after renal transplantation, relative to an appropriate reference as described herein. In some embodiments, the particular effect may be or comprise, for example, a number of dialysis sessions of from about 0.25 to about 1.5, from about 0.5 to about 1.25, or from about 0.75 to about 1.25 fewer per subject within 28 days after renal transplantation, relative to an appropriate reference as described herein. In some embodiments, the particular effect may be or comprise, for example, the mean number of dialysis sessions per subject within 28 days after renal transplantation of about 1.0.

In some embodiments, a particular effect may be or comprise, for example, a particular mean duration of dialysis within 28 days after renal transplantation. In some embodiments, the particular effect may be or comprise, for example, a shorter mean duration of dialysis within 28 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration). In some embodiments, the particular effect may be or comprise, for example, a mean duration of dialysis of less than about 10 days, less than about 9 days, or less than about 8 days within 28 days after renal transplantation. In some embodiments, the particular effect may be or comprise, for example, a mean duration of dialysis of from about 6 days to about 10 days, from about 6.5 days to about 9.5 days, or from about 7 days to about 8 days within 28 days after renal transplantation. In some embodiments, the particular effect may be or comprise, for example, a mean duration of dialysis of about 7.6 days within 28 days after renal transplantation. In some embodiments, the particular effect may be or comprise, for example, a mean duration of dialysis that is about 2.8, about 2.6, about 2.4, about 2.2, or about 2.0 days shorter than that of an appropriate reference as described herein. In some embodiments, the particular effect may be or comprise, for example, a mean duration of dialysis that is about 2.4 days shorter than that of an appropriate reference as described herein. In some embodiments, the particular effect may be or comprise, for example, a mean duration of dialysis that is from about 3.0 days to about 2.0 days, from about 2.8 days to about 2.2 days, or from about 2.6 days to about 2.2 days shorter than that of an appropriate reference as described herein.

In some embodiments, a particular effect may be or comprise, for example, a particular mean number of days a patient remains dialysis dependent within the first 30 days after renal transplantation. In some embodiments, a number of days a patient remains dialysis dependent is measured from the first day of treatment (i.e., within about 30 hours of renal transplantation) until either (i) the day of last dialysis session, wherein a last dialysis session is a dialysis session which is followed by 7 consecutive dialysis-free days, or (ii) Day 30. In some embodiments, the particular effect may be or comprise, for example, a fewer number of days a patient remains dialysis dependent within the first 30 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular mean length of hospitalization after renal transplantation. In some embodiments, the particular effect may be or comprise, for example, a shorter mean length of hospitalization after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration). In some embodiments, the particular effect may be or comprise, for example, a mean length of hospitalization after renal transplantation of less than about 11 days, about 10 days, about 9 days, or about 8 days. In some embodiments, the particular effect may be or comprise, for example, a mean length of hospitalization after renal transplantation of from about 6 days to about 11 days, from about 7 days to about 10 days, or from about 7 days to about 8 days. In some embodiments, the particular effect may be or comprise, for example, a mean length of hospitalization after renal transplantation of about 7.6 days. In some embodiments, the particular effect may be or comprise, for example, a mean length of hospitalization after renal transplantation of about 4.4 days, about 4.2 days, about 4.0 days, about 3.8 days, about 3.6 days, or about 3.4 days shorter than that of an appropriate reference, as described herein.

In some embodiments, a particular effect may be or comprise, for example, a particular incidence of graft failure within 12 months after renal transplantation. In some embodiments, the particular effect may be or comprise, for example, a lower incidence of graft failure within 12 months after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration). In some embodiments, the particular effect may be or comprise, for example, less than about 20%, about 15%, about 10%, or about 5% of subjects experiencing graft failure within 12 months after renal transplantation. In some embodiments, the particular effect may be or comprise, for example, from about 30% to about 0%, from about from about 20% to about 0%, from about 10% to about 0% or from about 5% to about 0% of subjects experiencing graft failure within 12 months after renal transplantation. In some embodiments, the particular effect may be or comprise, for example, about 0% of subject experiencing graft failure within 12 months after renal transplantation. In some embodiments, the particular effect may be or comprise, for example, a percentage of subjects experiencing graft failure that is about 20, about 25, about 30, or about 35 percentage points less than that of an appropriate reference as described herein.

In some embodiments, a particular effect may be or comprise, for example, a particular incidence of DGF, as assessed by proportion of patients requiring dialysis within 7 days after renal transplantation. In some embodiments, a particular effect may be or comprise, for example, a lower incidence of DGF, as assessed by proportion of patients requiring dialysis within 7 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular proportion of patients with primary non-function (PNF), defined as a continuous requirement for dialysis for at least 60 days after renal transplantation. In some embodiments, a particular effect may be or comprise, for example, a lesser proportion of patients with primary non-function (PNF), defined as a continuous requirement for dialysis for at least 60 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular proportion of patients with slow graft function (SGF), defined as having a SCr<3 mg/dL within the first 7 days after renal transplantation without dialysis required. In some embodiments, a particular effect may be or comprise, for example, a lesser proportion of patients with slow graft function (SGF), defined as having a SCr<3 mg/dL within the first 7 days after renal transplantation without dialysis required, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular proportion of patients with (1) PNF (defined as a continuous requirement for dialysis for at least 60 days after renal transplantation); (2) DGF (assessed by proportion of patients requiring dialysis within 7 days after renal transplantation); (3) SGF (defined as having a SCr<3 mg/dL within the first 7 days after renal transplantation without dialysis required); or (4) none of (1)-(3). In some embodiments, a particular effect may be or comprise, for example, a lesser proportion of patients with (1) PNF (defined as a continuous requirement for dialysis for at least 60 days after renal transplantation); (2) DGF (assessed by proportion of patients requiring dialysis within 7 days after renal transplantation); and/or (3) SGF (defined as having a SCr<3 mg/dL within the first 7 days after renal transplantation without dialysis required), relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular proportion of patients with acute rejection, e.g., at a particular time point, e.g., after renal transplantation. In some embodiments, a particular effect may be or comprise, for example, a lesser proportion patients with acute rejection, e.g., at a particular time point, e.g., after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular level of C-reactive protein (CRP), e.g., at a particular time point, e.g., within about 1 day or about 3 days after renal transplantation. In some embodiments, a particular effect may be or comprise, for example, a particular decrease from baseline of CRP levels, e.g., at a particular time point, e.g., within about 3 days after renal transplantation. In some embodiments, a particular effect may be or comprise, for example, a greater decrease from baseline of CRP levels, e.g., at a particular time point, e.g., within about 3 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular level of neutrophil gelatinase-associated lipocalin (NGAL), e.g., at a particular time point, e.g., within about 1 day or about 3 days after renal transplantation. In some embodiments, a particular effect may be or comprise, for example, a particular decrease from baseline of NGAL levels, e.g., at a particular time point, e.g., within about 3 days after renal transplantation. In some embodiments, a particular effect may be or comprise, for example, a greater decrease from baseline of NGAL levels, e.g., at a particular time point, e.g., within about 3 days after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, a particular effect may be or comprise, for example, a particular level of kidney injury molecule-1 (KIM-1), e.g., at a particular time point, e.g., after renal transplantation. In some embodiments, a particular effect may be or comprise, for example, a particular difference from baseline of KIM-1 levels, e.g., at a particular time point, e.g., after renal transplantation. In some embodiments, a particular effect may be or comprise, for example, a greater difference from baseline of KIM-1 levels, e.g., at a particular time point, e.g., after renal transplantation, relative to an appropriate reference as described herein (e.g., as is observed in a comparable population who has not received a composition providing Compound 1, which comparable population may, in some embodiments, have received a reference composition that is otherwise comparable but does not provide Compound 1 upon administration).

In some embodiments, the present disclosure encompasses the recognition that administration of a composition providing Compound 1 to particular patient populations may achieve certain desirable results. In some embodiments, a composition providing Compound 1 is administered to a particular patient population according to a regimen established to achieve a particular effect (e.g., at a particular time point).

In some embodiments, a particular effect in a population of subjects who have received a kidney from a donor after brain death (DBD) may be or comprise, for example, a particular incidence of achieving 1200 cc urine output over a 24 hour period within 28 days after renal transplantation. In some embodiments, the particular effect in a population of subjects who have received a kidney from a DBD may be or comprise, for example, a greater incidence of achieving 1200 cc urine output over a 24 hour period within 28 days after renal transplantation, relative to an appropriate reference (e.g., as is observed in a comparable population of subjects who have not received a kidney from a DBD).

The present disclosure also provides methods of administering Compound 1 (e.g., by administering a composition that comprises and/or delivers Compound 1 as described herein) to a subject or population of subjects in need thereof, regardless of the subject's malignancy status. Among other things, HGF/SF is known to stimulate c-MET (e.g., in injured organ tissues), which leads to activation of various cellular pathways, including, e.g., those involved in tissue repair. In addition, it is suspected that uncontrolled activation of c-MET can initiate tumorigenesis and/or stimulate tumor growth. As described above, Compound 1 is a HGF/SF mimetic, and as such, without wishing to be bound by theory, administration of Compound 1 under certain conditions might be expected to promote initiation or growth of cancer and/or other malignancies. Yet, experiments described herein (e.g., Example 5) demonstrate that, surprisingly, this may not be the case when Compound 1 is administered according to methods provided herein. Accordingly, the present disclosure encompasses the recognition that Compound 1 can be administered to a subject or population of subjects in need thereof, regardless of the subject's malignancy status. It will be appreciated that such insight may be applicable not only to methods of treating indications described herein (e.g., treating DGF, improving renal function after kidney transplantation, etc.), but also to methods of treating any indication for which Compound 1 therapy is suitable.

In some embodiments, the present disclosure provides methods comprising administering a composition providing Compound 1 (e.g., as provided herein) to a subject or population of subjects in need thereof, wherein the subject is suffering from an active malignancy or has suffered from a solid, metastatic or hematologic malignancy (e.g., within 5 years prior to administration of the composition). In some such embodiments, the subject has not suffered from a basal or squamous cell carcinoma of the skin that has been treated and/or removed. In some embodiments, the subject is suffering from or has suffered from glioma, colon cancer, or pancreatic cancer.

In some embodiments, the present disclosure provides methods comprising administering a composition providing Compound 1 (e.g., as provided herein) to a subject or population of subjects in need thereof, wherein the subject has not been assessed for an active malignancy or a history of a solid, metastatic or hematologic malignancy. In some embodiments, a subject has not be assessed for an active malignancy or a history of a solid, metastatic or hematologic malignancy if prior to administration of Compound 1 (e.g., within about 1 year, about 6 months, about 3 months, about 2 months, about 1 month, about 2 weeks, or about 1 week), the subject has not been questioned about and/or screened for an active malignancy or a history of a solid, metastatic or hematologic malignancy. In some embodiments, a subject has not been assessed for an active malignancy or a history of a solid, metastatic or hematologic malignancy if a decision to administer Compound 1 therapy (e.g., by a physician) did not depend upon questioning of and/or screening of the subject for an active malignancy or a history of a solid, metastatic or hematologic malignancy.

### Subjects to be Treated

In some embodiments, one or more subjects or populations selected to receive Compound 1 as described herein are characterized by one or more factors such as, for example, one or more of: presence of one or more risk factors for DGF, characteristic(s) of donor source, etc.

In some embodiments, a subject or population thereof has undergone renal transplantation. In some embodiments, a subject or population thereof has end-stage renal disease; alternatively or additionally, in some embodiments, a subject or population thereof has received and/or is receiving dialysis (e.g., chronic dialysis). In some embodiments, a subject or population thereof has not undergone a preemptive renal transplantation. In some embodiments, a subject or population thereof did not exhibit normal urine output, e.g., prior to renal transplantation; alternatively or additionally, in some embodiments, a subject or population thereof did not receive dialysis prior to renal transplantation. In some embodiments, a subject or population thereof has or had renal failure requiring hemodialysis or peritoneal dialysis initiated at least 3 months prior to renal transplantation.

In some embodiments, a subject or population thereof has undergone renal transplantation about 36 hours prior to administration of Compound 1 (e.g., within about 12 hours, about 18 hours, about 24 hours, about 30 hours, or about 36 hours of administration of Compound 1). In some embodiments, a subject or population thereof has undergone renal transplantation about 30 hours prior to administration of Compound 1 (e.g., within about 12 hours, about 18 hours, about 24 hours, or about 30 hours of administration of Compound 1). In some embodiments, blood flow was restored to an engrafted kidney during a subject or population's renal transplantation about 30 hours prior to administration of Compound 1 (e.g., within about 12 hours, about 18 hours, about 24 hours, or about 30 hours of administration of Compound 1).

In some embodiments, a subject or population thereof has undergone renal transplantation and is at risk of DGF. In some embodiments, a subject or population thereof who is at risk of DGF exhibits no urine output or an average urine output of less than 50 cc/h over at least 8 consecutive hours or exhibits normal urine output following transplantation that diminished to an average of less than 50 cc/h over at least 8 consecutive hours. In some embodiments, a subject or population thereof who is at risk of DGF exhibits a creatinine reduction ratio of less than 30% from pre-transplantation to 24 h after transplantation.

In some embodiments, a subject or population thereof has received a kidney from a healthy donor. In some embodiments, a subject or population thereof has received a kidney from a donor with a history of diabetes mellitus (e.g., Type I diabetes or Type II diabetes) or hypertension. In some embodiments, a subject or population thereof has received a kidney from a donor without a history of diabetes mellitus (e.g., Type I diabetes or Type II diabetes) or hypertension.

In some embodiments, a subject or population thereof has not previously undergone a renal transplantation. In some embodiments, a subject or population thereof has undergone only one renal transplantation.

In some embodiments, a subject or population thereof has received a kidney from a live donor. In some embodiments, a subject or population thereof has received a kidney from a deceased donor (i.e., a cadaveric donor). In some embodiments, a subject or population thereof has received a kidney from a donor after brain death (DBD). In some embodiments, a subject or population thereof has received a kidney from a donor after cardiac death (DCD).

In some embodiments, a subject or population thereof has received a kidney from a donor with a terminal serum creatinine concentration (SCr) of less than or equal to about 2.2 mg/dL.

In some embodiments, a subject or population thereof has received a kidney with a cold ischemia time of less than or equal to about 40 h. In some embodiments, a subject or population thereof has received a kidney with a cold ischemia time of less than or equal to about 30 h.

In some embodiments, a subject or population thereof has undergone a renal transplantation which utilized pulsatile machine perfusion (PMP). In some such embodiments, a subject or population thereof has received a kidney with a cold ischemia time of less than or equal to about 40 h.

In some embodiments, a subject or population thereof is not and will not be the recipient of multiple organ transplantation. In some embodiments, a subject or population thereof is not the recipient of a pediatric en-bloc kidney transplantation. In some embodiments, a subject or population thereof is not the recipient of an ABO-incompatible kidney. In some embodiments, a subject or population thereof is not the recipient of a kidney preserved by normothermic machine perfusion.

In some embodiments, a subject or population thereof did not have a measurable donor-specific antibody or positive cross-match requiring deviation from standard immunosuppressive therapy prior to renal transplantation.

In some embodiments, a subject or population thereof is not receiving and has not received a cytochrome P450 1A2 (CYP1A2) inhibitor, ciprofloxacin, or fluvoxamine. In some embodiments, a subject or population thereof is not receiving and has not received a cytochrome P450 1A2 (CYP1A2) inhibitor. In some embodiments, a subject or population thereof is not receiving and has not received ciprofloxacin or fluvoxamine.

In some embodiments, a subject or population thereof is female. In some such embodiments, a subject or population thereof had a negative pregnancy test prior to transplantation. In some such embodiments, a subject or population thereof is not breastfeeding. In some embodiments, a subject or population thereof is male.

In some embodiments, a subject or population thereof is at least 18 years of age.

In some embodiments, a subject or population thereof is not suffering from sepsis or an active bacterial infection. In some embodiments, a subject or population thereof does not have a history of a positive HIV test.

In some embodiments, a subject or population thereof has a dry weight of less than or equal to 120 kg. In some embodiments, a subject or population thereof has a BMI of less than 35. In some embodiments, a subject or population thereof has a BMI of less than 40.

In some embodiments, a subject or population is not suffering from or susceptible to a malignancy (e.g., an active malignancy). In some embodiments, a subject or population is not receiving treatment for a malignancy (e.g., an active malignancy). In some embodiments, a subject or population does not have a history of solid or hematological malignancies (e.g., within the past 5 years). In some embodiments, a subject or population is not suffering from or susceptible to a malignancy (e.g., an active malignancy) other than a basal or squamous cell carcinoma-in-situ of the skin that was diagnosed more than 2 years prior. In some embodiments, a subject or population thereof is not suffering from an active malignancy or has not suffered from a solid, metastatic or hematologic malignancy (e.g., within 5 years prior to administration of Compound 1 therapy). In some embodiments, a subject or population thereof has suffered from a basal or squamous cell carcinoma of the skin that has been treated and/or removed.

In some embodiments, a subject or population is suffering from or susceptible to a malignancy (e.g., an active malignancy). In some embodiments, a subject or population is receiving treatment for a malignancy (e.g., an active malignancy). In some embodiments, a subject or population has a history of solid or hematological malignancies (e.g., within the past 5 years). In some embodiments, a subject or population is suffering from or susceptible to a malignancy (e.g., an active malignancy) other than a basal or squamous cell carcinoma-in-situ of the skin that was diagnosed more than 2 years prior. In some embodiments, a subject or population thereof is suffering from an active malignancy or has suffered from a solid, metastatic or hematologic malignancy (e.g., within 5 years prior to administration of Compound 1 therapy). In some embodiments, a subject or population thereof has not suffered from a basal or squamous cell carcinoma of the skin that has been treated and/or removed. In some embodiments, a subject or population thereof is suffering from or has suffered from glioma, colon cancer, or pancreatic cancer.

In some embodiments, a subject or population thereof has not been assessed for an active malignancy or a history of solid or hematological malignancies. In some embodiments, a subject or population thereof has not been assessed for an active malignancy or a history of a solid, metastatic, or hematologic malignancy. In some embodiments, a subject or population thereof has an unknown malignancy status (i.e., an unknown medical history with respect to malignancies).

### Administration

In some embodiments, a composition providing Compound 1, as described herein, can be administered in accordance with methods (e.g., according to a regimen) provided herein.

In some embodiments, a composition providing Compound 1 is administered intravenously. In some embodiments, a composition providing Compound 1 is administered over about 10 min, about 20 min, about 30 min, or about 40 min. In some embodiments, a composition providing Compound 1 is administered intravenously in an amount suitable to provide about 0.5 mg/kg, about 1 mg/kg, about 2 mg/kg, about 3 mg/kg, about 4 mg/kg, about 6 mg/kg, or about 8 mg/kg Compound 1. In some embodiments, a composition providing Compound 1 is administered intravenously at an infusion rate suitable to provide about 0.5 mg/kg, about 1 mg/kg, about 2 mg/kg, about 3 mg/kg, about 4 mg/kg, about 6 mg/kg, or about 8 mg/kg Compound 1 over about 10 min, about 20 min, about 30 min, or about 40 min. In some embodiments, provided formulations are administered as an infusion over about 30 min in an amount suitable to provide about 2 mg/kg Compound 1.

In some embodiments, methods provided herein comprise periodic administration of Compound 1 (e.g., three infusions of Compound 1 separated by 24 (±2) hours). In some embodiments, methods provided herein comprise administration of one, two, three, four or five infusions of Compound 1 separated by a regular interval. In some such embodiments, a regular interval can be about 24 hours, about 30 hours, or about 36 hours.

In some embodiments, methods provided herein comprise periodic administration of Compound 1 throughout a course of treatment (e.g., a course of treatment of about 1, about 2, about 3, about 4, or about 5 days). In some embodiments, Compound 1 is administered once daily throughout a course of treatment (e.g., a course of treatment of about 1, about 2, about 3, about 4, or about 5 days).

In some embodiments, a composition providing Compound 1 is administered once daily for three days. In some embodiments, a composition providing Compound 1 is administered at 2 mg/kg once daily for three days. In some embodiments, a composition providing Compound 1 is administered once daily for four days. In some embodiments, a composition providing Compound 1 is administered at 2 mg/kg once daily for four days.

In some embodiments, a composition providing Compound 1 is first administered within about 12 hours, about 18 hours, about 24 hours, about 30 hours, about 36 hours, or about 42 hours of renal transplantation. In some embodiments, a composition providing Compound 1 is administered for a second time within about 20 hours, about 22 hours, about 24 hours, about 26 hours, or about 28 hours from the time of first administration. In some embodiments, a composition providing Compound 1 is administered for a third time within about 20 hours, about 22 hours, about 24 hours, about 26 hours, or about 28 hours from the time of second administration.

In some embodiments, the present disclosure provides a method comprising intravenously administering to a subject or population a formulation comprising:
about 6 mg/mL Compound 1;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, the present disclosure provides a method comprising intravenously administering to a subject or population a formulation comprising:
about 6 mg/mL Compound 1;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

In some embodiments, the present disclosure provides a method comprising steps of: (i) providing a first formulation of Compound 1; (ii) diluting the first formulation with normal saline to give a second formulation of Compound 1; and (iii) administering the second formulation to a subject or population in need thereof (e.g., as described herein). In some such embodiments, a first formulation of Compound 1 is more concentrated (e.g., 10 mg/mL) than a second formulation of Compound 1 (e.g., 6 mg/mL). In some embodiments, provided methods further comprise diluting the first formulation under aseptic conditions. In some embodiments, provided methods further comprise diluting the first formulation within 1 day, 2 days, or 3 days prior to administering the second formulation.

In some embodiments, a first formulation of Compound 1 comprises:
about 10 mg/mL Compound 1;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline; and
   a second formulation of Compound 1 comprises:
about 6 mg/mL Compound 1;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, a first formulation of Compound 1 comprises:
about 10 mg/mL Compound 1;
about 50% (w/v) polyethylene glycol (e.g., polyethylene glycol 300);
about 10% (w/v) polysorbate (e.g., polysorbate 80); and
one or more aqueous components selected from phosphate buffered saline and normal saline; and
   a second formulation of Compound 1 comprises:
about 6 mg/mL Compound 1;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

### EXEMPLARY EMBODIMENTS

The following numbered embodiments, while non-limiting, are exemplary of certain aspects of the disclosure:

A1. A method comprising:
administering to a population of subjects who have undergone renal transplantation and are at risk of delayed graft function
a composition providing (E)-3-[2-(2-thienyl)vinyl]-1H-pyrazole,
according to a regimen established to achieve one or more of:
   (i) a greater mean estimated glomerular filtration rate at about 6 months or about 12 months after renal transplantation;
   (ii) a greater mean increase in estimated glomerular filtration rate from baseline at about 6 months or about 12 months after renal transplantation;
   (iii) a lesser mean serum creatinine concentration at about 6 months or about 12 months after renal transplantation; and
   (iv) a greater mean decrease in serum creatinine concentration from baseline at about 6 months or about 12 months after renal transplantation,
      relative to a comparable reference population.
      A2. The method of embodiment A1, wherein the reference population has not received the composition providing (E)-3-[2-(2-thienyl)vinyl]-1H-pyrazole.
      A3. The method of embodiment A1 or A2, wherein the reference population has received an otherwise comparable reference composition that does not provide (E)-3-[2-(2-thienyl)vinyl]-1H-pyrazole.
      A4. The method of any one of embodiments A1-A3, wherein the regimen has been established to achieve a greater mean estimated glomerular filtration rate at about 6 months or about 12 months after renal transplantation, relative to a comparable reference population.
      A5. The method of any one of embodiments A1-A4, wherein the regimen has been established to achieve a greater mean estimated glomerular filtration rate at about 6 months and about 12 months after renal transplantation, relative to a comparable reference population.
      A6. The method of any one of embodiments A1-A4, wherein the regimen has been established to achieve a greater mean estimated glomerular filtration rate at about 12 months after renal transplantation, relative to a comparable reference population.
      A7. The method of any one of embodiments A1-A6, wherein the estimated glomerular filtration rate was calculated using the CKD-EPI equation.
      A8. The method of any one of embodiments A1-A7, wherein the regimen has been established to achieve a mean estimated glomerular filtration rate of from about 45 mL/min/1.73 m² to about 55 mL/min/1.73 m² at about 6 months after renal transplantation.
      A9. The method of any one of embodiments A1-A8, wherein the regimen has been established to achieve a mean estimated glomerular filtration rate of from about 45 mL/min/1.73 m² to about 55 mL/min/1.73 m² at about 12 months after renal transplantation.
      A10. The method of any one of embodiments A1-A9, wherein the regimen has been established to achieve a greater mean increase in estimated glomerular filtration rate from baseline at about 6 months or about 12 months after renal transplantation.

A11. The method of any one of embodiments A1-A10, wherein the regimen has been established to achieve a greater mean increase in estimated glomerular filtration rate from baseline at about 6 months and about 12 months after renal transplantation.

A12. The method of any one of embodiments A1-A11, wherein the regimen has been established to achieve a mean increase in estimated glomerular filtration rate from baseline of from about from about 36 mL/min/1.73 m² to about 45 mL/min/1.73 m² at about 6 months after renal transplantation.
A13. The method of any one of embodiments A1-A12, wherein the regimen has been established to achieve a mean increase in estimated glomerular filtration rate from baseline of from about from about 36 mL/min/1.73 m² to about 45 mL/min/1.73 m² at about 12 months after renal transplantation.
A14. The method of any one of embodiments A1-A13, wherein the regimen has been established to achieve a lesser mean serum creatinine concentration at about 6 months or about 12 months after renal transplantation, relative to a comparable reference population.
A15. The method of any one of embodiments A1-A14, wherein the regimen has been established to achieve a lesser mean serum creatinine concentration at about 6 months and about 12 months after renal transplantation, relative to a comparable reference population.
A16. The method of any one of embodiments A1-A15, wherein the regimen has been established to achieve a mean serum creatinine concentration of from about 1.55 mg/dL to about 1.85 mg/dL at about 6 months after renal transplantation.
A17. The method of any one of embodiments A1-A16, wherein the regimen has been established to achieve a mean serum creatinine concentration of from about 1.45 mg/dL to about 1.75 mg/dL at about 12 months after renal transplantation.
A18. The method of any one of embodiments A1-A17, wherein the regimen has been established to achieve a greater mean decrease in serum creatinine concentration from baseline at about 6 months or about 12 months after renal transplantation.
A19. The method of any one of embodiments A1-A18, wherein the regimen has been established to achieve a greater mean decrease in serum creatinine concentration from baseline at about 6 months and about 12 months after renal transplantation.
A20. The method of any one of embodiments A1-A19, wherein the regimen has been established to achieve a mean decrease in serum creatinine concentration from baseline of from about from about 6.1 mg/dL to about 6.5 mg/dL at about 6 months after renal transplantation.
A21. The method of any one of embodiments A1-A20, wherein the regimen has been established to achieve a mean decrease in serum creatinine concentration from baseline of from about from about 6.1 mg/dL to about 6.5 mg/dL at about 12 months after renal transplantation.
A22. The method of any one of embodiments A1-A21, wherein the regimen has further been established to achieve one or more of:
(v) a greater incidence of achieving 1200 cc urine output over a 24 hour period within 28 days after renal transplantation;
(vi) a shorter median time to achieve 1200 cc urine output over a 24 hour period within 28 days after renal transplantation;
(vii) a lesser mean number of dialysis sessions per subject within 28 days after renal transplantation;
(viii) a shorter mean duration of dialysis within 28 days after renal transplantation; (ix) a shorter mean length of hospitalization after renal transplantation;
(x) a lower incidence of graft failure within 12 months after renal transplantation;
(xi) a lesser mean serum creatinine concentration at about 7, 14, or 28 days after renal transplantation;
(xii) a greater mean decrease in serum creatinine concentration from baseline at about 7 days, about 14 days, or about 28 days after renal transplantation;
(xiii) a greater mean estimated glomerular filtration rate at about 7, 14, or 28 days after renal transplantation; and
(xiv) a greater mean increase in estimated glomerular filtration rate from baseline at about 7 days, about 14 days, or about 28 days after renal transplantation,
   relative to a comparable reference population.
   A23. The method of any one of embodiments A1-A22, wherein the regimen has further been established to achieve one or more of:
(xv) a greater proportion of patients with an estimated glomerular filtration rate greater than about 30 mL/min/1.73 m² at about 30 days, about 90 days, about 6 months or about 12 months after renal transplantation;
(xvi) a lesser proportion of patients with (1) PNF (defined as a continuous requirement for dialysis for at least 60 days after renal transplantation); (2) DGF (assessed by proportion of patients requiring dialysis within 7 days after renal transplantation); or (3) SGF (defined as having a SCr<3 mg/dL within the first 7 days after renal transplantation without dialysis required);
(xvii) a shorter mean length of hospitalization after renal transplantation; and
(xviii) a lesser number of days a patient remains dialysis dependent within the first 30 days after renal transplantation,
   relative to a comparable reference population.
   A24. A method comprising:
administering to a population of subjects who have undergone renal transplantation and are at risk of delayed graft function
a composition providing (E)-3-[2-(2-thienyl)vinyl]-1H-pyrazole,
according to a regimen established to achieve one or more of.
   (i) a greater mean estimated glomerular filtration rate at about 12 months after renal transplantation;
   (ii) a greater proportion of patients with an estimated glomerular filtration rate greater than about 30 mL/min/1.73 m² at about 30 days, about 90 days, about 6 months or about 12 months after renal transplantation;
   (iii) a lesser proportion of patients with (1) PNF (defined as a continuous requirement for dialysis for at least 60 days after renal transplantation); (2) DGF (assessed by proportion of patients requiring dialysis within 7 days after renal transplantation); or (3) SGF (defined as having a SCr<3 mg/dL within the first 7 days after renal transplantation without dialysis required);
   (iv) a shorter mean length of hospitalization after renal transplantation; and (v) a lesser number of days a patient remains dialysis dependent within the first 30 days after renal transplantation,
      relative to a comparable reference population.
      A25. The method of embodiment A24, wherein the reference population has not received the composition providing (E)-3-[2-(2-thienyl)vinyl]-1H-pyrazole.
      A26. The method of embodiment A24 or A25, wherein the reference population has received an otherwise comparable reference composition that does not provide (E)-3-[2-(2-thienyl)vinyl]-1H-pyrazole.
      A27. The method of any one of embodiments A24-A26, wherein the regimen has been established to achieve a greater mean estimated glomerular filtration rate at about 12 months after renal transplantation, relative to a comparable reference population.
      A28. The method of any one of embodiments A1-A27, wherein the population of subjects has undergone renal transplantation with a cadaveric kidney.
      A29. The method of embodiment A28, wherein the population of subjects has undergone renal transplantation with a kidney from a donor after brain death.
      A30. The method of embodiment A29, wherein the donor had a history of diabetes mellitus and/or hypertension.
      A31. The method of embodiment A29, wherein the donor did not have a history of diabetes mellitus and/or hypertension.
      A32. The method of embodiment A28, wherein the population of subjects has undergone renal transplantation with a kidney from a donor after cardiac death.
      A33. The method of any one of embodiments A1-A27, wherein the population of subjects has undergone renal transplantation with a kidney from a live donor.
      A34. The method of any one of embodiments A1-A33, wherein the composition providing (E)-3-[2-(2-thienyl)vinyl-1H-pyrazole is administered within about 36 h after renal transplantation.
      A35. The method of any one of embodiments A1-A34, wherein the composition providing (E)-3-[2-(2-thienyl)vinyl-1H-pyrazole is administered within about 30 h after renal transplantation.
      A36. The method of any one of embodiments A1-A35, wherein the composition providing (E)-3-[2-(2-thienyl)vinyl-1H-pyrazole is administered intravenously.
      A37. The method of any one of embodiments A1-A34, wherein the composition providing (E)-3-[2-(2-thienyl)vinyl-1H-pyrazole is administered in a dose of about 2 mg/kg once a day for three days.
      A38. The method of any one of embodiments A1-A37, wherein the composition providing (E)-3-[2-(2-thienyl)vinyl]-1H-pyrazole comprises:
   (E)-3-[2-(2-thienyl)vinyl]-1H-pyrazole;
   polyethylene glycol; and
   polysorbate.
      A39. The method of any one of embodiments A1-A38, wherein the regimen has further been established to achieve a lower stage on the National Kidney Foundation's CKD predictive scale as measured by estimated glomerular filtration rate at about 6 months or about 12 months after renal transplantation, relative to a comparable population.
      A40. The method of any one of embodiments A1-A39, wherein the regimen has further been established to achieve Stage 3A on the National Kidney Foundation's CKD predictive scale as measured by estimated glomerular filtration rate at about 6 months or about 12 months after renal transplantation, relative to a comparable population.
      A41. The method of any one of embodiments A1-A40, wherein the population of subjects is not suffering from an active malignancy and has not suffered from a solid, metastatic or hematologic malignancy.
      A42. The method of any one of embodiments A1-A41, wherein the population of subjects has suffered from a basal or squamous cell carcinoma of the skin that has been treated and/or removed.
      A43. The method of any one of embodiments A1-A42, wherein the population of subjects has not suffered from a solid, metastatic or hematologic malignancy within 5 years prior to administration of the composition providing (E)-3-[2-(2-thienyl)vinyl]-1H-pyrazole.
      A44. The method of any one of embodiments A1-A40, wherein the population of subjects is suffering from an active malignancy or has suffered from a solid, metastatic or hematologic malignancy.
      A45. The method of any one of embodiments A1-A40 and A44, wherein the population of subjects has not suffered from a basal or squamous cell carcinoma of the skin that has been treated and/or removed.
      A46. The method of any one of embodiments A1-A40, A44 and A45, wherein the population of subjects has suffered from a solid, metastatic or hematologic malignancy within 5 years prior to administration of the composition providing (E)-3-[2-(2-thienyl)vinyl]-1H-pyrazole.
      A47. The method of any one of embodiments A1-A40, wherein the population of subjects has not been assessed for an active malignancy or a history of a solid, metastatic or hematologic malignancy.
      A48. A method comprising:
administering a composition providing (E)-3-[2-(2-thienyl)vinyl]-1H-pyrazole to a subject in need thereof,
wherein the subject is suffering from an active malignancy or has suffered from a solid, metastatic or hematologic malignancy.
   A49. The method of embodiment A48, wherein the subject has not suffered from a basal or squamous cell carcinoma of the skin that has been treated and/or removed.
   A50. The method of embodiment A48 or A49, wherein the subject has suffered from a solid, metastatic or hematologic malignancy within 5 years prior to administration of the composition providing (E)-3-[2-(2-thienyl)vinyl]-1H-pyrazole.
   A51. A method comprising:
administering a composition providing (E)-3-[2-(2-thienyl)vinyl]-1H-pyrazole to a subject in need thereof,
wherein the subject has not been assessed for an active malignancy or a history of a solid, metastatic or hematologic malignancy.
   A52. The method of any one of embodiments A48-A51, wherein the composition providing (E)-3-[2-(2-thienyl)vinyl-1H-pyrazole is administered intravenously.
   A53. The method of any one of embodiments A48-A52, wherein the composition providing (E)-3-[2-(2-thienyl)vinyl-1H-pyrazole is administered in a dose of about 2 mg/kg.
   A54. The method of any one of embodiments A48-A53, wherein the composition providing (E)-3-[2-(2-thienyl)vinyl-1H-pyrazole is administered once daily.
   A55. The method of any one of embodiments A48-A54, wherein the composition providing (E)-3-[2-(2-thienyl)vinyl-1H-pyrazole is administered once daily for three or four days.
   A56. The method of any one of embodiments A48-A55, wherein the composition providing (E)-3-[2-(2-thienyl)vinyl]-1H-pyrazole comprises:
   (E)-3-[2-(2-thienyl)vinyl]-1H-pyrazole;
   polyethylene glycol; and
   polysorbate.
      A57. The method of any one of embodiments A1-A56, wherein the composition providing (E)-3-[2-(2-thienyl)vinyl]-1H-pyrazole was prepared by a process comprising a step of combining:
   crystalline (E)-3-[2-(2-thienyl)vinyl]-1H-pyrazole;
   polyethylene glycol (e.g., polyethylene glycol 300);
   polysorbate (e.g., polysorbate 80); and
   one or more aqueous components (e.g., phosphate buffered saline and/or normal saline) to obtain the composition.
      A58. The method of embodiment A57, wherein the crystalline (E)-3-[2-(2-thienyl)vinyl]-1H-pyrazole is Form A.
      B1. A composition providing (E)-3-[2-(2-thienyl)vinyl]-1H-pyrazole for use in administering to a subject or population of subjects who have undergone renal transplantation.
      B2. A composition providing (E)-3-[2-(2-thienyl)vinyl]-1H-pyrazole for use in a method of improving kidney graft function in a subject or population of subjects who have undergone renal transplantation.
      B3. A composition providing (E)-3-[2-(2-thienyl)vinyl]-1H-pyrazole for use in a method of treating delayed graft function in a subject or population of subjects who have undergone renal transplantation.
      B4. A composition providing (E)-3-[2-(2-thienyl)vinyl]-1H-pyrazole for use in a method of increasing life expectancy in a subject or population of subjects who have undergone renal transplantation.
      B5. The composition for use of any one of embodiments B1-B4, wherein the composition is administered intravenously.
      B6. The composition for use of any one of embodiments B1-B5, wherein the composition is administered in a dose of 2 mg/kg.
      B7. The composition for use of any one of embodiments B1-B6, wherein the composition is administered once daily.
      B8. The composition for use of any one of embodiments B1-B7, wherein the composition is administered once daily for three days.
      B9. The composition for use of any one of embodiments B1-B8, wherein the composition is administered within about 30 h after renal transplantation.
      B10. The composition for use of any one of embodiments B1-B9, wherein the composition comprises:
   about 6 mg/mL (E)-3-[2-(2-thienyl)vinyl]-1H-pyrazole;
   about 20% (w/v) to about 40% (w/v) polyethylene glycol 300;
   about 5% (w/v) to about 15% (w/v) polysorbate 80; and
   one or more aqueous components selected from phosphate buffered saline and normal saline.
      B11. The composition for use of any one of embodiments B1-B10, wherein the subject is at risk of delayed graft function.
      B12. The composition for use of embodiment B11, wherein the subject has an average urine output of less than about 50 mL/hour over any consecutive 8 hours in the first 24 hours after transplantation.
      B13. The composition for use of any one of embodiments B1-B12, wherein the subject has received a kidney from a deceased donor.
      B14. The composition for use of any one of embodiments B1-B13, wherein the composition is administered to a population of subjects according to a regimen established to achieve one or more of:
   (i) a greater mean estimated glomerular filtration rate at about 6 months or about 12 months after renal transplantation;
   (ii) a greater mean increase in estimated glomerular filtration rate from baseline at about 6 months or about 12 months after renal transplantation;
   (iii) a lesser mean serum creatinine concentration at about 6 months or about 12 months after renal transplantation; and
   (iv) a greater mean decrease in serum creatinine concentration from baseline at about 6 months or about 12 months after renal transplantation,
      relative to a comparable reference population.
      B15. The composition for use of embodiment B14, wherein the reference population has received an otherwise comparable reference composition that does not provide (E)-3-[2-(2-thienyl)vinyl]-1H-pyrazole.
      B16. The composition for use of embodiment B14 or B15, wherein the regimen has been established to achieve a greater mean estimated glomerular filtration rate at about 12 months after renal transplantation, relative to a comparable reference population.
      B17. The composition for use of any one of embodiments B14-B16, wherein the estimated glomerular filtration rate was calculated using the CKD-EPI equation.
      B18. The composition for use of any one of embodiments B14-B17, wherein the regimen has been established to achieve a mean estimated glomerular filtration rate of from about 45 mL/min/1.73 m² to about 55 mL/min/1.73 m² at about 12 months after renal transplantation.
      B19. The composition for use of any one of embodiments B14-B18, wherein the regimen has been established to achieve a greater mean increase in estimated glomerular filtration rate from baseline at about 12 months after renal transplantation.
      B20. The composition for use of any one of embodiments B14-B19, wherein the regimen has been established to achieve a mean increase in estimated glomerular filtration rate from baseline of from about from about 36 mL/min/1.73 m² to about 45 mL/min/1.73 m² at about 12 months after renal transplantation.
      B21. The composition for use of any one of embodiments B14-B20, wherein the regimen has been established to achieve a lesser mean serum creatinine concentration at about 12 months after renal transplantation, relative to a comparable reference population.
      B22. The composition for use of any one of embodiments B14-B21, wherein the regimen has been established to achieve a mean serum creatinine concentration of from about 1.45 mg/dL to about 1.75 mg/dL at about 12 months after renal transplantation.
      B23. The composition for use of any one of embodiments B14-B22, wherein the regimen has been established to achieve a greater mean decrease in serum creatinine concentration from baseline at about 12 months after renal transplantation.
      B24. The composition for use of any one of embodiments B14-B23, wherein the regimen has been established to achieve a mean decrease in serum creatinine concentration from baseline of from about from about 6.1 mg/dL to about 6.5 mg/dL at about 12 months after renal transplantation.
      B25. The composition for use of any one of embodiments B14-B24, wherein the regimen has further been established to achieve one or more of:
   (v) a greater incidence of achieving 1200 cc urine output over a 24 hour period within 28 days after renal transplantation;
   (vi) a shorter median time to achieve 1200 cc urine output over a 24 hour period within 28 days after renal transplantation;
   (vii) a lesser mean number of dialysis sessions per subject within 28 days after renal transplantation;
   (viii) a shorter mean duration of dialysis within 28 days after renal transplantation; (ix) a shorter mean length of hospitalization after renal transplantation;
   (x) a lower incidence of graft failure within 12 months after renal transplantation; (xi) a lesser mean serum creatinine concentration at about 7, 14, or 28 days after renal transplantation;
   (xii) a greater mean decrease in serum creatinine concentration from baseline at about 7 days, about 14 days, or about 28 days after renal transplantation;
   (xiii) a greater mean estimated glomerular filtration rate at about 7, 14, or 28 days after renal transplantation; and
   (xiv) a greater mean increase in estimated glomerular filtration rate from baseline at about 7 days, about 14 days, or about 28 days after renal transplantation,
      relative to a comparable reference population.
      B26. The composition for use of any one of embodiments B14-B25, wherein the regimen has further been established to achieve one or more of:
   (xv) a greater proportion of patients with an estimated glomerular filtration rate greater than about 30 mL/min/1.73 m² at about 30 days, about 90 days, about 6 months or about 12 months after renal transplantation;
   (xvi) a lesser proportion of patients with (1) PNF (defined as a continuous requirement for dialysis for at least 60 days after renal transplantation); (2) DGF (assessed by proportion of patients requiring dialysis within 7 days after renal transplantation); or (3) SGF (defined as having a SCr<3 mg/dL within the first 7 days after renal transplantation without dialysis required);
   (xvii) a shorter mean length of hospitalization after renal transplantation; and (xviii) a lesser number of days a patient remains dialysis dependent within the first 30 days after renal transplantation,
      relative to a comparable reference population.
      B27. The composition for use of any one of embodiments B1-B26, wherein the subject or population of subjects is suffering from an active malignancy or has suffered from a solid, metastatic or hematologic malignancy.
      B28. The composition for use of any one of embodiments B1-B27, wherein the subject or population of subjects has not been assessed for an active malignancy or a history of a solid, metastatic or hematologic malignancy.
      B29. A composition providing (E)-3-[2-(2-thienyl)vinyl]-1H-pyrazole for use in administering to a subject who is suffering from an active malignancy or has suffered from a solid, metastatic or hematologic malignancy.
      B30. The composition for use of embodiment B29, wherein the subject has not suffered from a basal or squamous cell carcinoma of the skin that has been treated and/or removed.
      B31. The composition for use of embodiment B29 or B30, wherein the subject has suffered from a solid, metastatic or hematologic malignancy within 5 years prior to administration of the composition providing (E)-3-[2-(2-thienyl)vinyl]-1H-pyrazole.
      B32. A composition providing (E)-3-[2-(2-thienyl)vinyl]-1H-pyrazole for use in administering to a subject who has not been assessed for an active malignancy or a history of a solid, metastatic or hematologic malignancy.
      B33. The composition for use of any one of embodiments B29-B32, wherein the composition is administered intravenously.
      B34. The composition for use of any one of embodiments B29-B33, wherein the composition is administered in a dose of about 2 mg/kg.
      B35. The composition for use of any one of embodiments B29-B34, wherein the composition is administered once daily.
      B36. The composition for use of any one of embodiments B29-B35, wherein the composition is administered once daily for three or four days.
      B37. The composition for use of any one of embodiments B29-B36, wherein the composition comprises:
   about 6 mg/mL (E)-3-[2-(2-thienyl)vinyl]-1H-pyrazole;
   about 20% (w/v) to about 40% (w/v) polyethylene glycol 300;
   about 5% (w/v) to about 15% (w/v) polysorbate 80; and
   one or more aqueous components selected from phosphate buffered saline and normal saline.
      B38. The composition for use of any one of embodiments B1-B37, wherein the composition providing (E)-3-[2-(2-thienyl)vinyl]-1H-pyrazole was prepared by a process comprising a step of combining:
   crystalline (E)-3-[2-(2-thienyl)vinyl]-1H-pyrazole;
   polyethylene glycol (e.g., polyethylene glycol 300);
   polysorbate (e.g., polysorbate 80); and
   one or more aqueous components (e.g., phosphate buffered saline and/or normal saline) to obtain the composition.
      B39. The composition for use of embodiment B38, wherein the crystalline (E)-3-[2-(2-thienyl)vinyl]-1H-pyrazole is Form A.

Many modifications and variations of the embodiments described herein may be made without departing from the scope, as is apparent to those skilled in the art. The specific embodiments described herein are offered by way of example only.

### EXAMPLES

### Example 1. A Hepatocyte Growth Factor Mimetic Improves Outcomes in Patients at Risk of DGF: A Randomized Double-Blind Placebo-Controlled Phase 2 Human Clinical Trial

### Overall Study Design and Plan

The study was a multi-center, randomized, double-blind, placebo-controlled, Phase 2 study.

Subjects undergoing kidney transplant were eligible for enrollment if they met at least one of the following conditions: 1) inadequate urine output (oliguria), defined as <50 cc/h urine output for eight consecutive hours over the first 24 h post-transplant; or 2) low creatinine clearance, defined as a creatinine reduction ratio of <30% from pre-transplantation to 24 h post-transplant. In addition, the kidney donor had to have a terminal creatinine ≤2.2 mg/dL.

Eligible subjects were randomized 2:1 to receive 2 mg/kg Compound 1 or placebo via 30 min intravenous (IV) infusion. Initial infusion occurred within 36 h of renal transplant (Day 1), with two subsequent infusions at 24 (±2) h intervals. An initial cohort of subjects was infused over 10 to 12 minutes. Based upon site feedback, the protocol was amended and the remaining subjects were infused over 30 min.

Subjects were monitored during initial hospitalization, with subsequent clinical visits on Days 7, 14, and 28. Subjects were contacted by phone on Days 5, 6, and 8 to 13. Following completion of the study on Day 28, long-term data on graft survival and renal function were collected approximately 6 and 12 months after renal transplantation.

During a subject's stay in the hospital (Days 1 to 3 and, if applicable, Day 4), blood samples were collected for determination of immunosuppressant levels and for pharmacokinetic (PK) analyses of Compound 1 and its metabolites. The subject's renal output over 24 h was assessed daily for 14 days after first study drug infusion and again on Day 28. Other assessments included standard safety measures, blood and urine biomarkers of renal function, and 24 h urine creatinine clearance.

### Selection of Study Population

Subjects were assessed for eligibility according to the following criteria:
1. Males and females ≥18 years of age
2. Had renal transplantation due to end-stage renal disease (ESRD) requiring chronic dialysis
3. Study drug could be administered within 36 h after transplantation
4. Received kidney from a healthy donor, or a donor with history of diabetes mellitus or hypertension
5. Donor terminal serum creatinine (SCr)≤2.2 mg/dL
6. No urine output, or average urine output of <50 cc/h over eight or more consecutive hours, or normal urine output following transplantation that diminished to an average of <50 cc/h over eight or more consecutive hours; or creatinine reduction ratio <30% from pre-transplantation to 24 h after transplant
7. Reason for low urine output was unlikely due to structural changes. If clinically indicated, an ultrasound was performed.
8. Dry weight ≤120 kg, body mass index (BMI)<35
9. Women of child bearing potential had a negative serum pregnancy test prior to transplantation
10. Women of child bearing potential (including perimenopausal women who had a menstrual period within one year) agreed to use two forms of an effective birth control regimen, including at least one-barrier method, during the 28-day study period. Men agreed to use condoms during the 28-day study period.
11. In the opinion of the investigator, the subject was capable of understanding and complying with the protocol
12. Subjects signed the informed consent document prior to performance of any study-related procedure including screening procedure

Subjects who met any of the following criteria were excluded from study participation:
1. Subject with normal urine output and not requiring dialysis prior to renal transplantation (i.e., had pre-emptive renal transplantation)
2. Signs and symptoms of volume depletion
3. Recipient of multiple organ transplantation or scheduled for multiple organ transplantation
4. Recipient of pediatric en-bloc kidney transplantation
5 Recipient of kidney with cold ischemia time >40 h
6. Had measurable donor-specific antibody or positive cross-match requiring deviation from standard immunosuppressive therapy
7. Was participating in or had participated in an investigational drug or medical device study within 30 days or five half-lives, whichever was longer, prior to enrollment into this study
8. Concurrent sepsis or active bacterial infection
9. Had an active malignancy or history of solid, metastatic or hematologic malignancy with the exception of basal or squamous cell carcinoma of the skin that had been removed
10. Women of child bearing potential who were breast feeding
11. History of positive human immunodeficiency virus (HIV) test
12. History of rheumatoid arthritis
13. Subjects who required the cytochrome P450 1A2 (CYP1A2) inhibitors, ciprofloxacin, and/or fluvoxamine (Luvox©)
14. Subject was unwilling or unable to comply with the protocol or to cooperate fully with the investigator or the site personnel
15. Subject was not deemed medically stable for the study in the opinion of the investigator or the subject's primary nephrologist

Infusions of study drug were pre-specified to be discontinued if a subject:
Withdrew consent; or
Developed a serious adverse event (SAE) attributable to study drug following administration of study drug; or
Had a >3-fold increase in aspartate aminotransferase (AST) or alanine aminotransferase (ALT) from pre-infusion to post-infusion of study drug. If AST or ALT was >3-fold higher than the pre-infusion value, the laboratory test could be repeated the same day to verify an increase. If AST or ALT still was >3-fold greater than the value prior to infusion, study drug was not to be administered.
   In addition, the investigator or medical monitor could decide at any time that infusion of study drug be canceled or delayed if a repeat administration placed the subject at risk for an adverse event (AE).

### Treatment

The active treatment was 2 mg/kg Compound 1 administered in a 30 min IV infusion. The first infusion was administered within 36 h after transplant, with two subsequent infusions at 24±2 h intervals. An initial cohort of subjects was infused over 10 to 12 min. Based upon site feedback, the protocol was amended and the remaining subjects were infused over 30 min. The volume of infusion was controlled by the infusion pump, based upon the subject's baseline weight (0.33 mL/kg) with a maximum infusion of 40 mL. Placebo was normal saline (United States Pharmacopeia [USP]) administered at the same intervals and volumes (0.33 mL/kg).

Eligible subjects were randomized 2:1 to receive either 2 mg/kg Compound 1 or placebo. Sites were provided a set of ordered envelopes with treatment assignment and the pharmacist prepared either active treatment or placebo based upon the ordered card. Subject enrollment was initially stratified by source of donor kidney: 1) without history of diabetes mellitus or hypertension, and 2) with history of diabetes mellitus and/or hypertension. This stratification was subsequently removed.

The dose regimen used in this study (2 mg/kg Compound 1) was selected based on data from nonclinical efficacy studies, as well as clinical studies assessing PK and safety in healthy volunteers and renal failure patients on hemodialysis. Preclinical studies determined that c-Met receptor upregulation occurs within 6 to 48 h following renal injury, and that Compound 1 administration within 24 h following renal ischemia injury improves renal outcome and attenuates mortality at doses up to 2 mg/kg. A dose escalation study in healthy volunteers, and a PK and safety study in patients on and off dialysis, confirmed 2 mg/kg as the optimal dose. Animal studies showed that repeat administration of Compound 1 was associated with improved renal outcomes. In a Phase 1 study of healthy subjects and a clinical study in subjects with renal failure on and off hemodialysis, IV infusions of Compound 1 were well tolerated at doses up to 6 mg/kg, the maximal practical dose.

### Other Medications

All concomitant medications, including dose, were recorded at study entry. Use of new concomitant medications or changes in dose of concomitant medications were recorded until the final study visit. The dose and dosing regimen of all prescriptions and non-prescription therapies and medications were documented.

Subjects could receive concomitant immunosuppressive regimens used as part of standard-of-care by the clinical center. Based upon the known metabolism, serum levels of cyclosporine, tacrolimus, and sirolimus were not expected to be affected by co-administration of study drug. Serum concentrations of immunosuppressants other than MPA were assayed on Days 1 to 4, prior to the morning or evening dose of immunosuppressant and prior to daily dosing of Compound 1. Both Compound 1 and MPA (the active metabolite of MMF) are metabolized by glucuronidation and the potential for drug-drug interaction has not been determined. For the trough concentration assay, blood samples were drawn within 1 h prior to the morning or evening dose of MMF/other immunosuppressant on Day 1, and 1 h prior to the morning dose of MMF/other immunosuppressant on Days 2 to 4.

Based on in vitro data, Compound 1 was considered both a substrate and an inhibitor of CYP1A2. Drugs utilizing the CYP1A2 enzyme were avoided, if possible, on days that study drug was administered and for the 24 h period after the last infusion. Caffeine intake (e.g., tea, coffee, and caffeine-containing drinks) was prohibited within 3 h before and up to 3 h after infusion. In addition, potent CYP1A2 inhibitors ciprofloxacin and fluvoxamine were prohibited during treatment and up to 24 h following the last infusion of study drug.

### Efficacy Variables

The primary efficacy variable was time (in days) until production of ≥1200 cc of urine over a 24 h period. The following secondary outcomes were also measured:
Mean total daily urine output through Day 14
Change from baseline in urine production at Days 2 to 14
Mean serum creatinine (SCr) at Days 3, 7, 10, 14, and 28 and Months 6 and 12
Mean measured 24-h creatinine clearance at Days 3, 7, 14, and 28
Mean serum levels of the biomarkers C-reactive protein (CRP) and neutrophil gelatinase-associated lipocalin (NGAL) at Days 1 and 3
Incidence of DGF, defined as the initiation of dialysis, through Day 7 after transplantation
Number of dialysis sessions through Days 7, 14, and 28
Length of hospitalization following transplantation
Acute rejection episodes
Graft survival at 6 and 12 months post-transplant

In addition, three post hoc analyses were performed:
Mean estimated glomerular filtration rate (eGFR) at screening, Day 3, Day 7, Day 14, Day 28, Month 6 and Month 12 by study arm
Descriptive analysis of duration of dialysis during first 28 days post-transplant by study arm
Log rank test of time to graft failure over the first 12 months after transplant by study arm

Certain efficacy variables were measured as described in the following paragraphs:

Total Daily Urine Output: During hospitalization, daily total urine output for each 24 h period was recorded. Following discharge, subjects recorded their total urine output in a diary each day through Day 14, and again on Day 28. The total urine output was recorded daily and obtained from the subject either by phone or through a scheduled study visit.

24-hour Urine Collection for Creatinine Clearance: Four 24-h urine samples were collected to measure creatinine clearance on Days 3, 7, 14, and 28. The subjects were provided handouts on 24 h urine collections and instructed to collect their urine for a 24 h period before the scheduled visits on Days 3, 7, 14, and 28. The 24-h urine samples were tested for sodium, potassium, chloride, and creatinine. Day 3 was selected as the first time point following transplantation to collect urine for creatinine clearance, since subjects in the study were likely to have unstable SCr at earlier time points due to acute renal injury post-transplantation. If the SCr levels remained unstable at the time of urine collection, clearance was estimated by averaging the SCr at the start and at the end of the collection.

Urine Analysis: A urine sample was collected on Days 14 and 28 to assess for signs of persistent inflammation in the urinary tract. Subjects with recent transplantation may have active sediments and variable findings on microscopic analysis. Subjects with inflammatory findings on urine analysis such as the presence of leukocyte esterase, ≥2-5 white blood cells (WBCs) per high power field, or leukocyte casts were evaluated for causes, such as infection. In subjects with new or increased findings of inflammation on Day 28, a follow-up urine analysis was performed within seven to ten days after the Day 28 evaluation.

Biomarkers: Inflammatory (C-reactive protein; CRP) and renal function (neutrophil gelatinase-associated lipocalin; NGAL) serum assays were performs. One 5 mL sample of blood was collected using heparin as an anticoagulant on Days 1 (pre-dose), 2 (pre-dose), 3 (pre-dose), 4 (if subject remained in hospital), 7, 14, and 28. Urine samples (10 mL each) were collected in the morning on the same days. The total volume and time of collection were recorded for each sample.

Rejection Episodes: On each study day, the investigator recorded any episodes of acute rejection, including how they were identified, documented, and treated.

Dialysis: On Days 1, 2, 3, 4, 5, 6, 7, 10, 14 and 28, the investigator recorded whether a patient received dialysis. Data were retrospectively collected on all dialysis sessions that occurred during the first 28 days.

Graft Survival and Renal Function: Approximately 6 and 12 months after the kidney transplant, the clinical site assessed subjects for graft survival and renal function. eGFR was derived from SCr for all timepoints using the MDRD4 formula. (See Levey, A. S., et al. Ann. Intern. Med. 2006; 145:247-54.)

Adverse Events: An AE was any unfavorable and unintended diagnosis, symptom, sign (including an abnormal laboratory finding), syndrome, or disease which either occurred during the study (if absent at baseline), or appeared to worsen (if present at baseline). The investigator monitored subjects for the occurrence of AEs throughout their participation in the study. All AEs, regardless of seriousness or relationship to study drug, including those occurring during the screening period (where applicable), were recorded. Whenever possible, symptoms were grouped as a single syndrome or diagnosis. The investigator was to specify the date of onset, maximal intensity, corrective therapy given, outcome, and their opinion regarding if there was a reasonable possibility that the AE was caused by the study drug. The causal relationship of an AE to the study drug was the best estimate of the investigator at the time of reporting. AEs were graded as mild, moderate, or severe based on the intensity of symptoms and the degree of limitation of usual daily activities.

Clinical Laboratory Assessments: Samples of venous blood were collected for the measurement of hematology and chemistry parameters at Screening and Days 2, 3, 4, 7, 14, and 28. Hematology parameters included hematocrit, hemoglobin, total leukocyte count, differential blood count (neutrophils, lymphocytes, monocytes, eosinophils, basophils), and platelet count. Chemistry parameters included glucose, sodium, potassium, chloride, bicarbonate, blood urea nitrogen (BUN), creatinine, calcium, phosphorus, albumin, AST, ALT, gamma-glutamyl transpeptidase (GGT), alkaline phosphatase, creatine kinase, lactate dehydrogenase (LDH), and total and direct bilirubin. Venous blood was drawn at Screening from female subjects of childbearing potential for pregnancy testing if results prior to transplantation were not available. Laboratory results were also evaluated by the investigator to determine if any abnormal results were clinically significant for an individual and were considered an AE.

MPA and Immunosuppressant Drug Levels: Samples of venous blood were collected daily on Days 1 to 4 for the analysis of levels of MPA (in subjects receiving the immunosuppressant MMF) and other immunosuppressants (cyclosporine, tacrolimus, or sirolimus; unless the subject had been discharged from the hospital before Day 4). The blood samples were drawn within 1 h prior to the morning or evening dose of MMF/other immunosuppressant on Day 1, and 1 h prior to the morning dose of MMF/other immunosuppressant on Days 2 to 4. The time of blood sampling, the time of administration of the previous MMF or immunosuppressant dose, and the previous MMF or immunosuppressant dose level were recorded in source documents and in the case report form (CRF).

### Disposition of Subjects

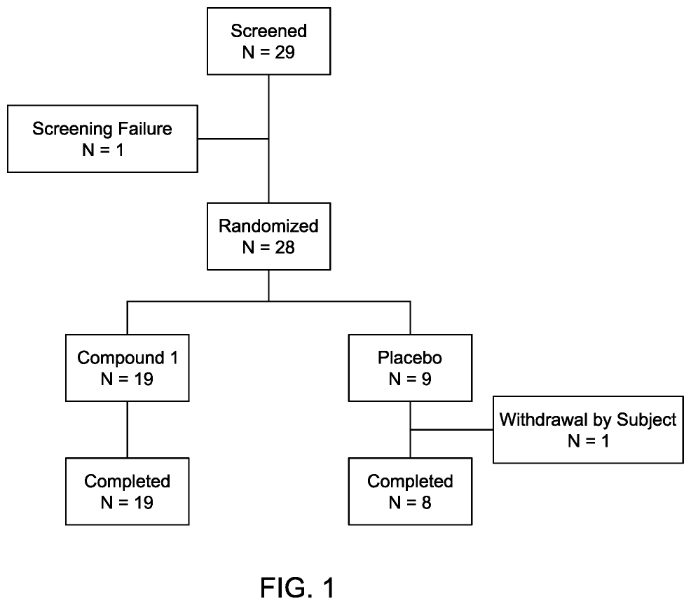

FIG. 1 shows the disposition of subjects. Twenty-nine subjects were screened and consented to participate in the study. One subject was judged by their physician to be too medically fragile to participate and was withdrawn from participation before randomization. The remaining 28 subjects were randomized: 19 to Compound 1 and 9 to placebo. Of the 28 subjects randomized, one subject in the placebo arm withdrew consent after the second infusion and did not complete the study. Withdrawal occurred after a moderate left bundle branch block, assessed as unrelated to the study product. All other subjects completed the study. The mean±SD duration of the study across subjects was 29.0±7.79 days (range: 4 to 50 days).

Seven subjects were retrospectively identified as not meeting all eligibility criteria, but received study drug and subsequently completed the study. Three subjects in the Compound 1 arm received their initial infusions more than 28 h after transplantation: at 29 h 14 min; 29 h 14 min; 30 h 20 min. One subject in the placebo arm received the first infusion within the prescribed 36 h window (35 h 35 min), but there was a note in the subject record stating that the initial infusion time was extended from 36 h to 38 h.

One subject in the Compound 1 arm was identified as having low urine output that a physician determined may have been due to structural issues associated with subsequent, non-kidney-related surgery. One subject in each study arm was discovered to have a history of prostate cancer, 3 years (Compound 1 arm) and 18 years (placebo arm) before enrollment. One subject in the placebo arm received a donor kidney with terminal creatinine ≤2.2 mg/dL.

There were 12 deviations from the planned dosing regimen. In the Compound 1 arm, there were six dosing deviations in four subjects. One subject failed to receive an infusion on Day 2 due to issues with dialysis that resulted in arm pain. One subject had Day 3 infusion delayed outside the 24-hour window. One subject had Day 3 infusion stopped and restarted. One subject had reduced infusion time on Day 3, while one subject had increased infusion times on Days 1 and 3. In the placebo arm, there were five dosing deviations in three subjects. One subject had infusions stopped and restarted on Days 1, 2, and 3. One subject had Day 3 infusion delayed to Day 5. One subject did not have their line flushed after infusion on Day 1.

### Demographic Baseline Characteristics

The demographic characteristics of the subjects in the clinical trial are shown in Table 1. Compared to the placebo arm, subjects in the Compound 1 arm were younger (mean±SD=54.7±13.65 vs 65.7±12.84 years), more likely to be female (21.1% vs 11.1%), and more likely to be black or African American (42.1% vs 22.2%).

TABLE 1
Demographics

| | Compound 1 | Placebo | Total |
|---|---|---|---|
| | (N = 19) | (N = 9) | (N = 28) |
| Age at signing informed | | | |
| consent (years) | | | |
| Mean (SD) | 54.7 (13.65) | 65.7 (12.84) | 58.3 (14.14) |
| Median | 52.0 | 65.0 | 58.5 |
| Min, Max | 29, 75 | 46, 84 | 29, 84 |
| Gender, n (%) | | | |
| Male | 15 (78.9) | 8 (88.9) | 23 (82.1) |
| Female | 4 (21.1) | 1 (11.1) | 5 (17.9) |
| Of childbearing potential | 1 (25.0) | 0 | 1 (20.0) |
| Post-menopausal | 1 (25.0) | 1 (100.0) | 2 (40.0) |
| Surgically sterile | 2 (50.0) | 0 | 2 (40.0) |
| Race, n (%) | | | |
| White | 9 (47.4) | 5 (55.6) | 14 (50.0) |
| Black or African | 8 (42.1) | 2 (22.2) | 10 (35.7) |
| American | | | |
| Asian | 1 (5.3) | 1 (11.1) | 2 (7.1) |
| American Indian or | 1 (5.3) | 0 (0.0) | 1 (3.6) |
| Alaska Native | | | |
| Other | 0 (0.0) | 1 (11.1) | 1 (3.6) |

Abbreviations: Max = Maximum; Min = Minimum; SD = Standard deviation.

The baseline clinical characteristics and medical history of the subjects are shown in Table 2 and Table 3, respectively. Compared to subjects in the placebo arm, subjects in the Compound 1 arm were higher in body weight (Compound 1=90.92 kg; placebo=79.78 kg) and dry weight (Compound 1=86.85 kg; placebo=80.85 kg), as well as BMI (Compound 1=30.1; Placebo=28.0). The percentage of subjects who reported currently consuming alcohol was similar between study arms (Compound 1=26%; placebo=33%), though subjects in the Compound 1 arm who drank reported an average of 4.0±4.24 drinks per week, vs 1.7±1.15 in the placebo arm. Three subjects (15.8%) in the Compound 1 arm were current smokers vs none in the placebo arm.

TABLE 2
Baseline Clinical Characteristics

| | Compound 1 | Placebo | Total |
|---|---|---|---|
| | (N = 19) | (N = 9) | (N = 28) |
| Height (cm), n | 19 | 9 | 28 |
| Mean (SD) | 173.5 (9.25) | 168.2 (7.19) | 171.8 (8.87) |
| Median | 173.0 | 168.0 | 172.0 |
| Min, Max | 157, 191 | 154, 180 | 154, 191 |
| Body Weight (kg), n | 19 | 9 | 28 |
| Mean (SD) | 90.92 (17.108) | 79.78 (16.204) | 87.34 (17.350) |
| Median | 88.60 | 76.20 | 88.20 |
| Min, Max | 51.3, 124.2 | 57.1, 108.0 | 51.3, 124.2 |
| Dry Weight (kg), n | 15 | 6 | 21 |
| Mean (SD) | 86.85 (16.587) | 80.85 (17.955)¹ | 85.13 (16.760) |
| Median | 87.90 | 82.05 | 87.90 |
| Min, Max | 51.3, 118.0 | 57.1, 108.0 | 51.3, 118.0 |
| BMI (kg/m²), n | 19 | 9 | 28 |
| Mean (SD) | 30.11 (4.659) | 27.98 (3.980) | 29.42 (4.494) |
| Median | 30.73 | 27.00 | 30.52 |
| Min, Max | 19.3, 36.4 | 23.2, 33.3 | 19.3, 36.4 |
| Alcohol Consumption, n (%) | | | |
| Never Consumed | 5 (26.3) | 4 (44.4) | 9 (32.1) |
| Currently Consumed | 5 (26.3) | 3 (33.3) | 8 (28.6) |
| Ex-user | 9 (47.4) | 2 (22.2) | 11 (39.3) |
| Average Alcohol Consumption per | 2 | 3 | 5 |
| Week² | | | |
| Mean (SD) | 4.0 (4.24) | 1.7 (1.15) | 2.6 (2.61) |
| Smoking Habits, n (%) | | | |
| Currently Smoking | 3 (15.8) | 0 | 3 (10.7) |
| Ex-Smoker | 5 (26.3) | 2 (22.2) | 7 (25.0) |
| Non-Smoker | 11 (57.9) | 7 (77.8) | 18 (64.3) |

Abbreviations: BMI = Body mass index; Max = Maximum; Min = Minimum; SD = Standard deviation.
¹In the Placebo arm, mean dry weight exceeded mean weight due to incomplete dry weight data (n = 9 for wet weight, n = 6 for dry weight).
²1 unit of alcohol is equivalent to 1 ounce of hard liquor or 4 ounces of wine or 12 ounces of beer.

Medical history was generally similar between study arms (Table 3). Hypertension was nearly universal across both groups (Compound 1=94.7%; placebo=100.0%). Diabetes/hyperglycemia was highly prevalent in both arms (Compound 1=68.4%; placebo=66.7%). Prevalence of gastrointestinal disease, musculoskeletal, urogenital, eye, and other surgeries were similar between groups.

In a few areas, there was a higher prevalence of disease in the placebo vs Compound 1 arm. Though highly prevalent in both groups, cardiovascular disease was higher in the placebo arm (100.0%) vs Compound 1 (78.9%). This was driven by higher prevalence of atherosclerotic disease/procedures, arrhythmia, heart failure, valve disease/disorder, and myocardial infarct in the placebo arm. The incidence of neuropsychiatric disorders and respiratory disorders were higher in the placebo arm (55.6% vs 26.3%; 33.3% vs 21.1% respectively), but not driven by any specific diagnoses.

TABLE 3
Medical History

| | Compound 1 | Placebo | Total |
|---|---|---|---|
| | (N = 19) | (N = 9) | (N = 28) |
| Hypertension | 18 (94.7) | 9 (100.0) | 27 (96.4) |
| Diabetes and hyperglycemia | 13 (68.4) | 6 (66.7) | 19 (67.9) |
| Diabetes | 10 (52.6) | 5 (55.6) | 15 (53.6) |
| Hyperglycemia | 3 (15.8) | 1 (11.1) | 4 (14.3) |
| Cardiovascular | 15 (78.9) | 9 (100.0) | 24 (85.7) |
| Atherosclerotic disease/ | 5 (26.3) | 7 (77.8) | 12 (42.9) |
| procedure | | | |
| Hyperlipidemia | 7 (36.8) | 3 (33.3) | 10 (35.7) |
| Cardiomegaly/cardiomyopathy | 5 (26.3) | 3 (33.3) | 8 (28.6) |
| Arrythmia | 2 (10.5) | 5 (55.6) | 7 (25.0) |
| Heart failure | 3 (15.8) | 3 (33.3) | 6 (21.4) |
| Myocardial infarct | 2 (10.5) | 2 (22.2) | 4 (14.3) |
| Valve disease/disorder | 1 (5.3) | 2 (22.2) | 3 (10.7) |
| Deep vein thrombosis | 2 (10.5) | 0 (0.0) | 2 (7.1) |
| Other | 2 (10.5) | 2 (22.2) | 4 (14.3) |
| Gastrointestinal | 10 (52.6) | 5 (55.6) | 15 (53.6) |
| GERD | 6 (31.6) | 2 (22.2) | 8 (28.6) |
| Appendectomy | 2 (10.5) | 0 (0.0) | 2 (7.1) |
| Other | 7 (36.8) | 3 (33.3) | 10 (35.7) |
| Musculoskeletal | 7 (36.8) | 4 (44.4) | 11 (39.3) |
| Gout | 3 (15.8) | 3 (33.3) | 6 (21.4) |
| Arthritis | 4 (21.1) | 1 (11.1) | 5 (17.9) |
| Joint surgery | 3 (15.8) | 1 (11.1) | 4 (14.3) |
| Other | 2 (10.5) | 2 (22.2) | 4 (14.3) |
| Other Surgeries | 7 (36.8) | 4 (44.4) | 11 (39.3) |
| Tonsillectomy | 3 (15.8) | 1 (11.1) | 4 (14.3) |
| Amputation below knee | 1 (5.3) | 1 (11.1) | 2 (7.1) |
| Hernia | 0 (0.0) | 3 (33.3) | 3 (10.7) |
| Other | 3 (15.8) | 1 (11.1) | 4 (14.3) |
| Neuropsychiatric | 5 (26.3) | 5 (55.6) | 10 (35.7) |
| Neuropathy | 3 (15.8) | 1 (11.1) | 4 (14.3) |
| Depression | 2 (10.5) | 1 (11.1) | 3 (10.7) |
| Sleep disturbance | 1 (5.3) | 2 (22.2) | 3 (10.7) |
| Other | 0 (0.0) | 3 (33.3) | 3 (10.7) |
| Urogenital | 6 (31.6) | 3 (33.3) | 9 (32.1) |
| Vasectomy/hysterectomy | 3 (15.8) | 1 (11.1) | 4 (14.3) |
| Erectile dysfunction | 1 (5.3) | 2 (22.2) | 3 (10.7) |
| Other | 1 (5.3) | 1 (11.1) | 2 (7.1) |
| Liver | 5 (26.3) | 3 (33.3) | 8 (28.6) |
| Cholecystitis | 3 (15.8) | 3 (33.3) | 6 (21.4) |
| Other | 2 (10.5) | 1 (11.1) | 3 (10.7) |
| Respiratory (COPD, | 4 (21.1) | 3 (33.3) | 7 (25.0) |
| Asthma, infection) | | | |
| Eye | 5 (26.3) | 2 (22.2) | 7 (25.0) |
| Cataracts | 4 (21.1) | 0 (0.0) | 4 (14.3) |
| Diabetic retinopathy | 2 (10.5) | 2 (22.2) | 4 (14.3) |
| Other | 1 (5.3) | 0 (0.0) | 1 (3.6) |
| Other | 4 (21.1) | 0 (0.0) | 4 (14.3) |

Abbreviations: COPD = Chronic Obstructive Pulmonary Disease; GERD = Gastroesophageal Reflux Disease.
Data shown are n (%). Percentages are based on N.

As per protocol, all 28 randomized subjects received a renal transplant. The characteristics of donors and donor kidneys are presented in Table 4. The majority of kidneys in both groups were from donors after brain death (DBD) (Compound 1=68.4%; placebo=77.8%). However, there were slightly more donors after cardiac death (DCD) in the Compound 1 arm (Compound 1=21.1%; placebo=11.1%). Donors in the Compound 1 arm were younger (43.0±21.2 years) than donors in the placebo arm (56.3±10.8 years) and had a lower prevalence of diabetes/hypertension (Compound 1=63.2%; placebo=77.8%). The mean time from organ procurement to transplant was nearly identical between study arms (Compound 1=23.3 h; placebo=23.7 h), as was total transplant time (Compound 1=3.2 h; placebo=3.0 h).

TABLE 4
Donor/Kidney and Renal Transplantation Characteristics

| | Compound 1 | Placebo | Total |
|---|---|---|---|
| | (N = 19) | (N = 9) | (N = 28) |
| Donation Type, n (%) | | | |
| Donor by Brain Death | 13 (68.4) | 7 (77.8) | 20 (71.4) |
| Donor by Cardiac Death | 4 (21.1) | 1 (11.1) | 5 (17.9) |
| Live | 2 (10.5) | 0 | 2 (7.1) |
| Unknown | 0 | 1 (11.1) | 1 (3.6) |
| History of Diabetes | | | |
| Mellitus or Hypertension | | | |
| Yes | 12 (63.2) | 7 (77.8) | 19 (67.9) |
| No | 7 (36.8) | 2 (22.2) | 9 (32.1) |
| Donor Age (years), n | 19 | 8 | 27 |
| Mean (SD) | 43.0 (21.20) | 56.3 (10.77) | 46.9 (19.51) |
| Median | 49.0 | 59.0 | 51.0 |
| Min, Max | 0, 72 | 37, 68 | 0, 72 |
| Was a Renal Transplantation | 19 (100.0) | 9 (100.0) | 28 (100.0) |
| Performed? Yes | | | |
| Time from Organ Harvest | | | |
| to Transplant (h) | | | |
| n (%) | 17 (90) | 8 (89) | 25 (89) |
| Unknown/non-calculable, n (%) | 2 (10) | 1 (11) | 3 (11) |
| Mean (SD) | 23.3 (9.2) | 23.7 (10.3) | 23.4 (9.3) |
| Median | 27.1 | 25.2 | 27.1 |
| Min, Max | 5.9, 36.1 | 10.1, 37.3 | 5.9, 37.3 |
| Total Transplant Time (h) | | | |
| n (%) | 16 (84.2) | 8 (88.9) | 24 (85.7) |
| Unknown/non-calculable, n (%) | 3 (15.8) | 1 (11.1) | 4 (14.3) |
| Mean (SD) | 3.2 (1.59) | 3.0 (0.94) | 3.1 (1.39) |
| Median | 2.9 | 3.0 | 2.9 |
| Min, Max | 1, 7 | 1, 4 | 1, 7 |

Abbreviations: Max = Maximum; Min = Minimum; SD = Standard deviation.
Data shown are n (%). Percentages are based on N.

### Results

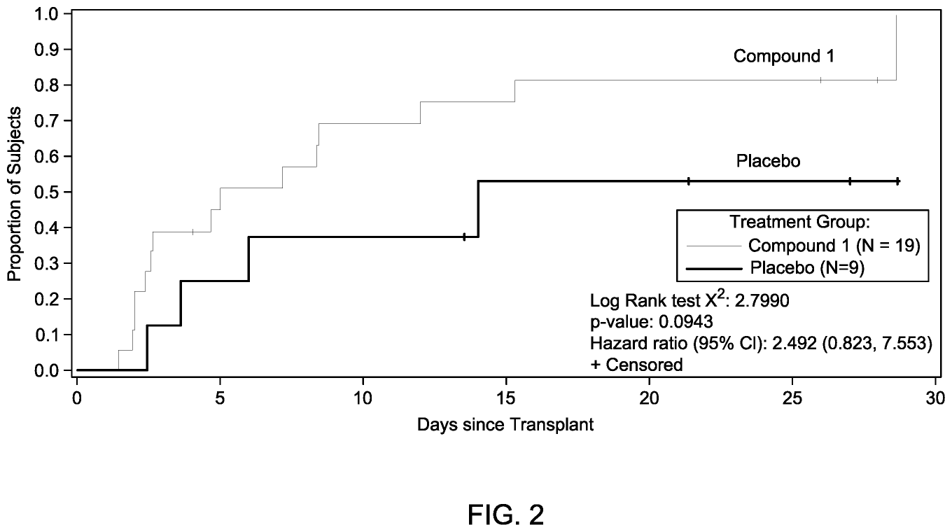

Time to Production of ≥1200 cc Urine over 24 Hours: Kaplan-Meier curves for time to production of ≥1200 cc urine over 24 h by study arm are presented in FIG. 2. At Day 28, 78.9% of subjects in the Compound 1 arm achieved ≥1200 cc urine output over 24 h vs 44.4% of subjects in the placebo arm (Table 5). The time from transplantation to production of ≥1200 cc of urine over 24 h was longer in the placebo arm than the Compound 1 arm (Log Rank test: χ²=2.799, p=0.094; Hazard ratio=2.49, 95% CI=0.82, 7.55).

The median number of days from transplantation to production of ≥1200 cc of urine over 24 h was 5 days for the Compound 1 arm (95% CI: 2.4, 12.0) and 14 days for the placebo arm (95% CI: 2.44, -; Table 5Table). The upper limit of the median could not be computed for the placebo arm, as <50% of subjects achieved the endpoint. Two subjects were excluded from the Survival Analysis as they reached 1200 cc urine over 24 h prior to the start of first infusion of investigational medicinal product.

TABLE 5
Time to Production of ≥1200 cc of Urine Over 24 Hours

| | Compound 1 | Placebo | Total |
|---|---|---|---|
| | (N = 19) | (N = 9) | (N = 28) |
| N (%) | 18 (94.7%) | 8 (88.9%) | 26 (92.9%) |
| Failed | 15 (78.9%) | 4 (44.4%) | 19 (67.9%) |
| Censored | 3 (15.8%) | 4 (44.44%) | 7 (25.0%) |
| Median Time in Days | 5.0 (2.4, 12.0) | 14.0 (2.4, -)¹ | 7.2 (2.6, 15.3) |
| (95% CI) | | | |

Abbreviations: CI = confidence interval
¹The upper limit could not be calculated as <50% of subjects achieved the outcome.

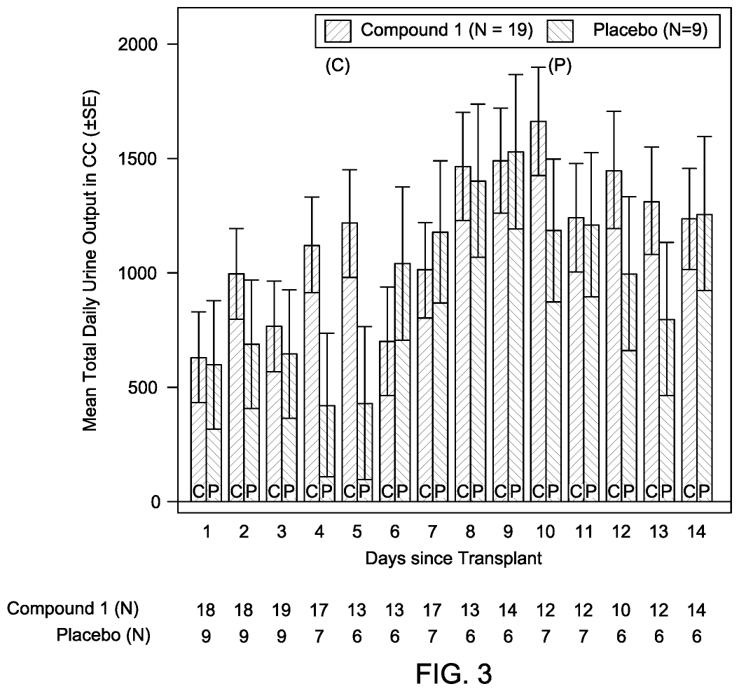

Mean Total Daily Urine Output: FIG. 3 shows MMRM LS mean and SE for total daily urine output (in cc) from post-transplant Day 1 to Day 14 by study arm. Day 1 urine output was higher (+90 cc) in the Compound 1 arm vs placebo arm. On 8 of the next 13 days, urine output was higher in the Compound 1 arm vs placebo: Day 2=+313 cc; Day 3=+127 cc; Day 4=+703 cc; Day 5=+782 cc; Day 8=+70 cc; Day 10=+471 cc; Day 12=+459 cc; Day 13=+529 cc. On days 9, 11 and 13, urine output differed by <50 cc between study arms. On Days 6 and 7, urine output was lower in the Compound 1 arm: Day 6=−353 cc; Day 7=−162 cc.

On Day 28, urine output was lower in the Compound 1 arm: Compound 1=972 cc; placebo=1652 cc; difference=−680 cc. However, this difference may have been largely driven by one placebo patient with urine output of 3500 cc. The removal of this subject reduced the difference between groups to 116 cc higher in the placebo arm.

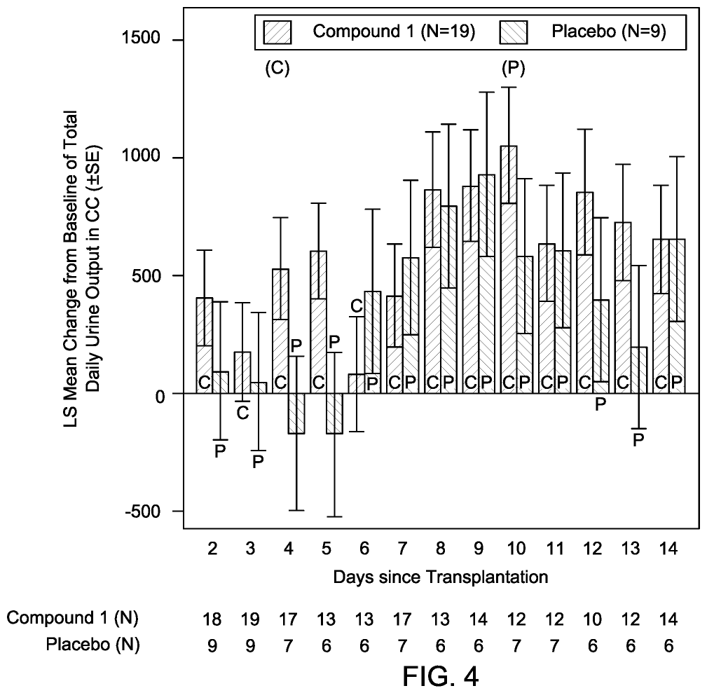

Change from Day 1 Urine Production through Day 14. FIG. 4 shows MMRM LS mean and SE for change from baseline (Day 1) in total daily urine output (in cc) from post-transplant Day 2 to Day 14 by study arm. On Days 2 to 5, the Compound 1 arm showed increases from baseline urine production (+408; +178; +531; +606, respectively), while the placebo arm showed small increases or decreases from baseline (+95; +51; −171; −176, respectively). On Days 8, 10, 12 and 13, the change from baseline total daily urine production was greater in the Compound 1 arm vs Placebo (+869 vs +799; +1056 vs +585; +860 vs +400; +729 vs +200, respectively). The differences between study arms was <50 cc on Days 9, 11 and 14. On Days 6 and 7, the Compound 1 arm showed less increase from baseline than placebo (+82 vs +436; +417 vs +479, respectively).

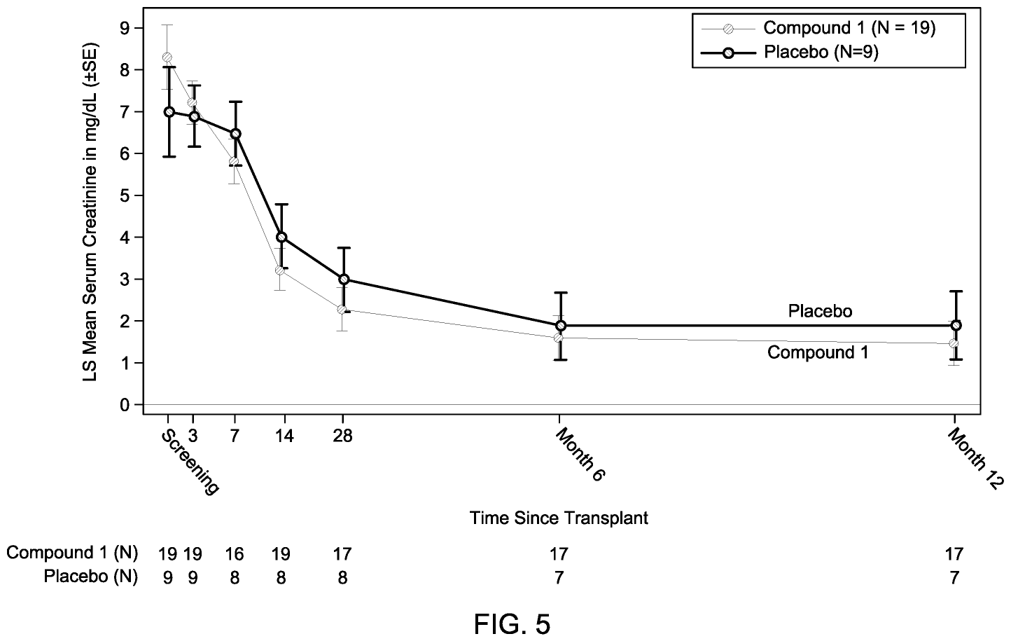

Serum Creatinine: FIG. 5 shows MMRM LS mean and SE for serum creatinine (mg/dL) over time by study arm. Compared to placebo, subjects in the Compound 1 arm had higher SCr at screening (Compound 1 mean=8.32; placebo mean=7.01; difference=+1.31 mg/dL) and Day 3 (Compound 1 mean=7.23, SE=0.50; placebo mean=6.90, SE=0.73; difference=+0.33 mg/dL). On Days 7, 14, 28, Months 6 and 12, subjects in the Compound 1 arm had lower SCr than subjects in the Placebo arm (difference=−0.67; −0.79; −0.70; −0.29; −0.43 mg/dL, respectively). Table 6 summarizes MMRM LS means and SE for SCr.

TABLE 6

| | | Compound 1 | Placebo |
|---|---|---|---|
| Time | Statistic | (N = 19) | (N = 9) |
| Day 7 | n | 16 | 8 |
| | LS Means (SE) | 5.82 (0.535) | 6.49 (0.765) |
| Day 14 | n | 19 | 8 |
| | LS Means (SE) | 3.24 (0.504) | 4.03 (0.765) |
| Day 28 | n | 17 | 8 |
| | LS Means (SE) | 2.29 (0.524) | 2.99 (0.765) |
| Month 6 | n | 17 | 7 |
| | LS Means (SE) | 1.60 (0.523) | 1.89 (0.801) |
| Month 12 | n | 17 | 7 |
| | LS Means (SE) | 1.48 (0.523) | 1.91 (0.801) |

Table 7 summarizes results of mean SCr change from baseline over time for the Compound 1 arm and placebo arm.

TABLE 7

| | | Compound 1 | Placebo |
|---|---|---|---|
| Time | Statistic | (N = 19) | (N = 9) |
| Day 7 | n | 16 | 8 |
| | LS Means (SE) | −2.05 (0.535) | −1.39 (0.765) |
| Day 14 | n | 19 | 8 |
| | LS Means (SE) | −4.63 (0.504) | −3.85 (0.765) |
| Day 28 | n | 17 | 8 |
| | LS Means (SE) | −5.59 (0.524) | −4.88 (0.765) |
| Month 6 | n | 17 | 7 |
| | LS Means (SE) | −6.27 (0.523) | −5.98 (0.801) |
| Month 12 | n | 17 | 7 |
| | LS Means (SE) | −6.40 (0.523) | −5.96 (0.801) |

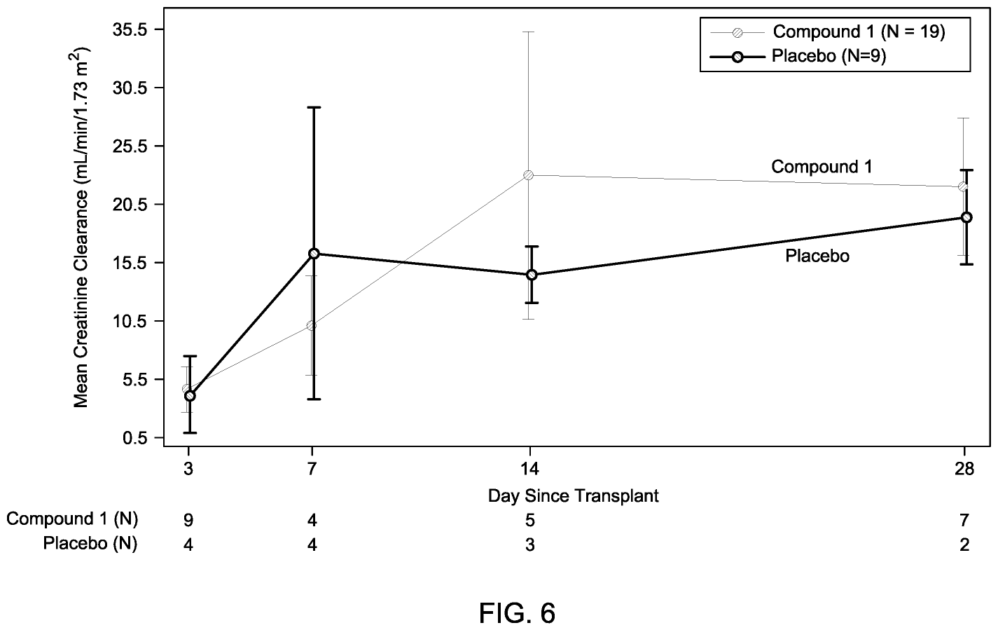

Mean 24-Hour Creatinine Clearance: FIG. 6 shows mean and SD of the mean for 24-hour urine creatinine clearance (mL/min/1.73 m²) over time by study arm. As shown, creatinine clearance was equivalent across study arms at Day 3 (Compound 1=4.7; placebo=4.2); higher in the placebo arm at Day 7 (Compound 1=10.1; placebo=16.3); and higher in the Compound 1 arm on Day 14 (Compound 1=23.0; placebo=14.5) and Day 28 (Compound 1=22.1; placebo=19.4).

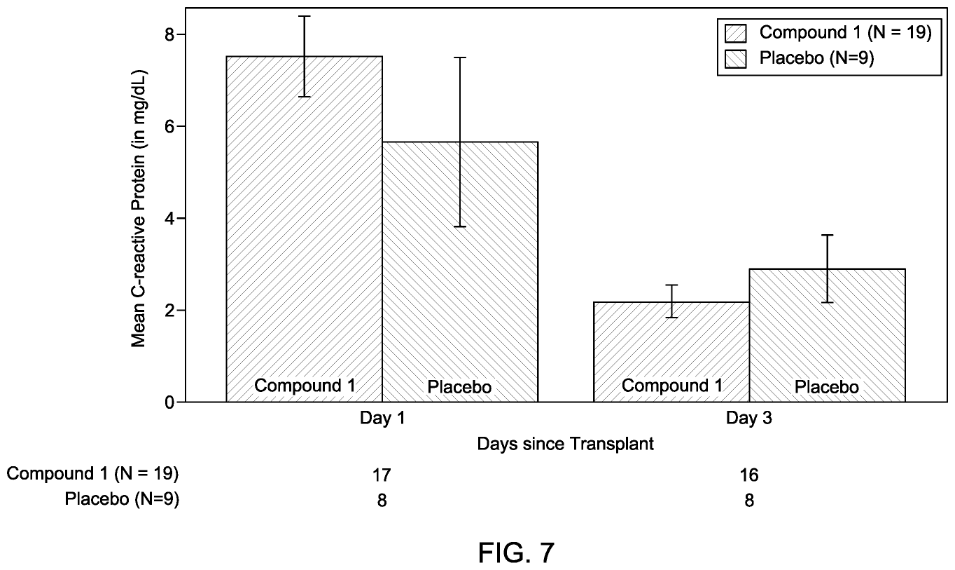

C-Reactive Protein (CRP): FIG. 7 shows serum CRP (mg/dL) by study arm at Day 1 and Day 3 post-transplant. On Day 1, CRP was higher in the Compound 1 arm (mean=7.5, SE=0.9) vs placebo (mean=5.7, SE=1.8). On Day 3, CRP was lower in the Compound 1 arm (mean=2.2, SE=0.4) vs placebo (mean=2.9, SE=0.7). There was a greater decrease in CRP in the Compound 1 arm (−68.6% from baseline) than in the placebo arm (−17.5% from baseline).

Neutrophil Gelatinase-Associated Lipocalin (NGAL): FIG. 8 shows NGAL (ng/mL) by study arm at Day 1 and Day 3 post-transplant. On Day 1, NGAL was higher in the Compound 1 arm (mean=1152, SE=121) vs placebo (mean=646, SE=102). On Day 3, NGAL was nearly equivalent between the Compound 1 arm (mean=614, SE=85) vs placebo (mean=571, SE=101). There was a greater decrease in NGAL in the Compound 1 arm (−43.5% from baseline) vs the placebo arm (−12.5% from baseline).

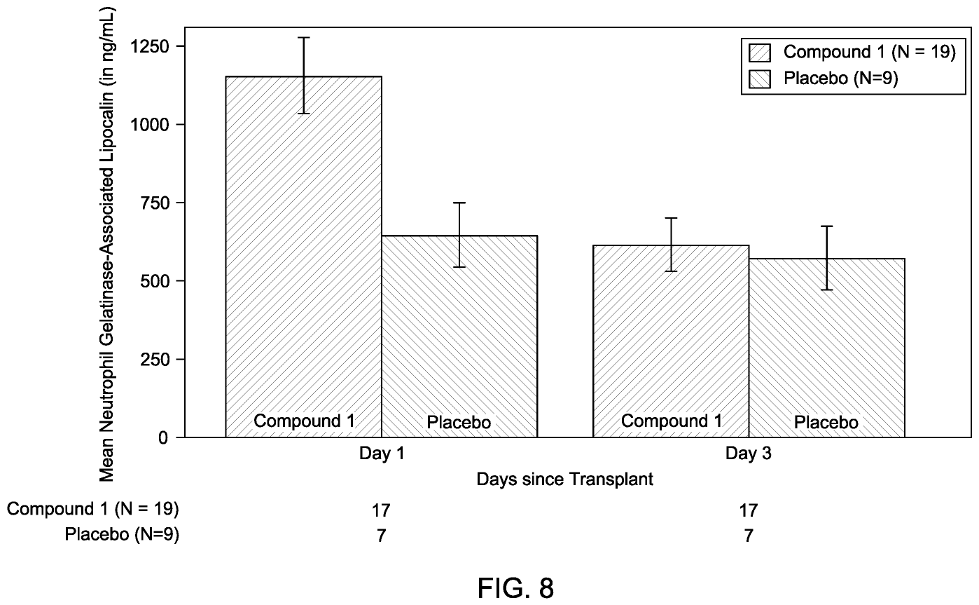

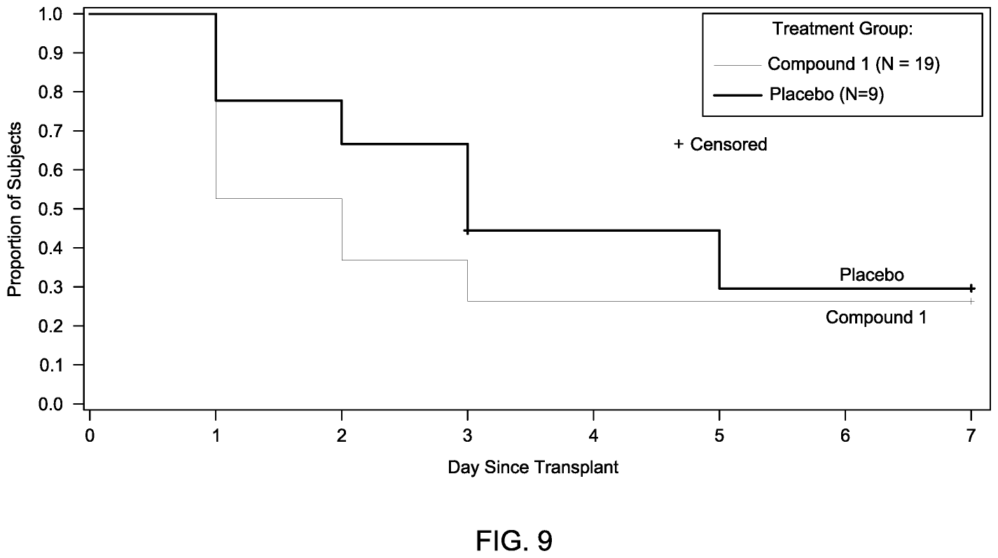

Incidence of DGF: DGF was defined by the initiation of dialysis during the first seven days after transplantation. As shown in FIG. 9, the percentage of subjects receiving dialysis in the first 7 days was slightly higher in the Compound 1 group (73.6%) vs placebo (66.6%). It should be noted that 10 of the 14 (71%) subjects in the Compound 1 arm who progressed to DGF had their first dialysis session before study drug was administered, versus 2 of 6 (33%) subjects in the placebo arm. All patients who received dialysis had their first session within the first 7 days after transplantation. The reason listed for dialysis for most subjects was “transplant DGF” or “DGF”. Other reasons included uremia, anuria, metabolic acidosis, hyperkalemia, fluid overload, and end stage renal disease. An AE was associated with the dialysis session due to DGF for three subjects.

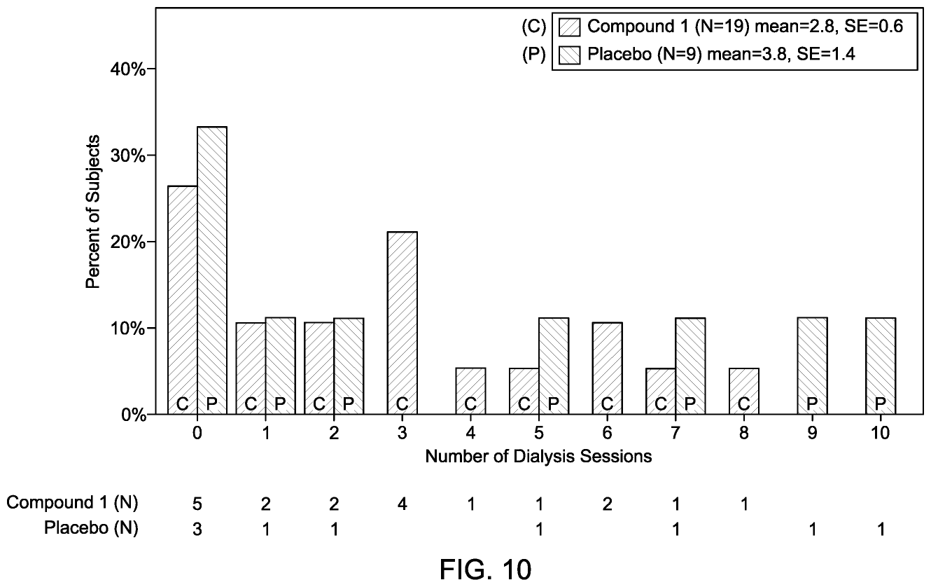

Number of Dialysis Sessions through Day 28: The cumulative number of dialysis sessions through Day 28 by treatment group are shown in FIG. 10. As shown, on average there were 1.0 more dialysis sessions during the first 28 days after transplant in the placebo arm (mean=3.8, SE=1.4) versus the Compound 1 arm (mean=2.8, SE=0.6).

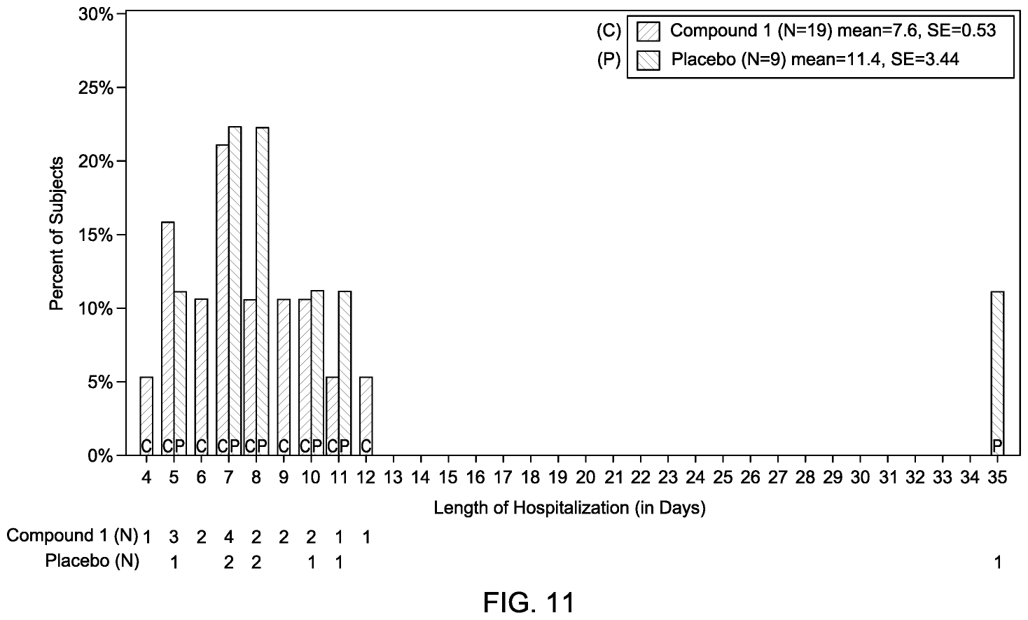

Length of Hospitalization Following Transplantation: FIG. 11 shows the mean length of transplant hospitalization (in days) by treatment arm. The mean length of hospitalization was 7.6 days (SE=0.5) for the Compound 1 arm and 11.4 days (SE=3.4) for the placebo arm, a difference of 3.8 days less for the Compound 1 arm. It should be noted that there was an outlier in the placebo arm with a hospitalization length of 35 days. Removing this patient from the analysis reduced the incremental hospital days for placebo to 0.5 days.

Number of Acute Rejection Episodes: There was no incidence of acute rejection in either study arm.

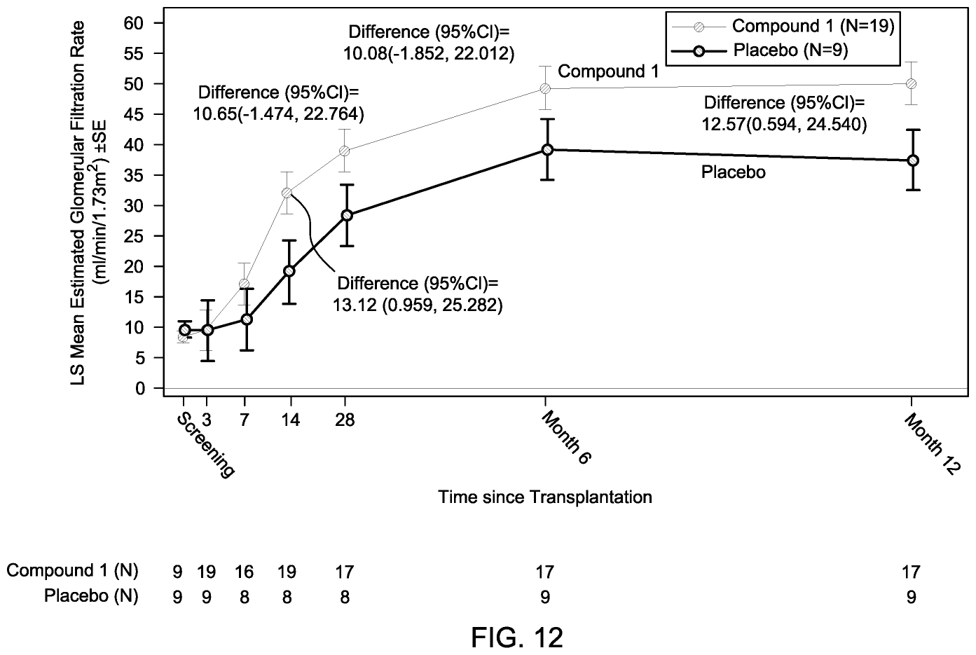

Mean Estimated Glomerular Filtration Rate (eGFR): FIG. 12 shows mean and SE from a mixed effects repeated measures model with an autoregressive covariance matrix for eGFR (mL/min/1.73 m²) by study arm at screening, Day 3, Day 7, Day 14, Day 28, Month 6 and Month 12. Two subjects in the placebo arm experienced graft failure and resumed maintenance dialysis. Their 6-month and 12-month eGFR values were set to zero. One subject in the placebo arm had 12-month eGFR collected at Month 15; this value was included in the 12-month analysis.

As shown, eGFR was higher in the Compound 1 arm vs placebo arm on Day 14 (Compound 1 mean=32.1, SE=3.4; placebo=20.6, SE=5.1); Day 28 (Compound 1 mean=39.0, SE=3.5; placebo=30.6, SE=5.1); Month 6 (Compound 1 mean=50.0, SE=3.5; placebo=39.5, SE=5.0); and Month 12 (Compound 1 mean=50.1, SE=3.5; placebo=37.8, SE=5.0). At Month 12, eGFR was 25% higher in the Compound 1 vs placebo. The difference between groups in difference of LS means from baseline at Day 14, Day 28, Month 6, and Month 12 were 13.1 (95% CI=0.96, 25.28), 10.7 (95% CI=−1.47, 22.76), 10.1 (95% CI=−1.85, 22.01), and 12.6 (95% CI=0.60, 24.54) mL/min/1.73 m² respectively. The p-values from the MMRM at Days 14, 28, Month 6, and Month 12 were p=0.035; p=0.085; p=0.097 and p=0.040, respectively.

TABLE 8

| | | Compound 1 | Placebo |
|---|---|---|---|
| Time | Statistic | (N = 19) | (N = 9) |
| Screening | n | 19 | 9 |
| | Mean | 8.43 | 9.65 |
| | SD | 4.390 | 4.128 |
| | Median | 7.48 | 9.11 |
| | Min, Max | 3.02, 22.20 | 4.93, 16.42 |
| Day 7 | n | 16 | 8 |
| | Mean | 15.11 | 11.96 |
| | SD | 12.303 | 6.454 |
| | Median | 12.85 | 9.99 |
| | Min, Max | 4.09, 53.71 | 5.71, 22.12 |
| | LS Means difference | 8.25 (3.499) | 2.46 (5.163) |
| | from baseline (SE) | | |
| Day 14 | n | 19 | 8 |
| | Mean | 32.08 | 20.61 |
| | SD | 20.372 | 9.346 |
| | Median | 28.20 | 19.82 |
| | Min, Max | 4.38, 90.49 | 7.46, 34.70 |
| | LS Means difference | 23.36 (3.399) | 10.24 (5.136) |
| | from baseline (SE) | | |
| Day 28 | n | 17 | 8 |
| | Mean | 38.96 | 30.60 |
| | SD | 20.792 | 15.532 |
| | Median | 34.37 | 28.35 |
| | Min, Max | 9.08, 90.49 | 11.14, 53.47 |
| | LS Means difference | 30.26 (3.455) | 19.61 (5.071) |
| | from baseline (SE) | | |
| Month 6 | n | 17 | 9 |
| | Mean | 49.97 | 39.49 |
| | SD | 20.580 | 27.365 |
| | Median | 51.87 | 51.02 |
| | Min, Max | 18.40, 90.49 | 0.00, 68.75 |
| | LS Means difference | 40.47 (3.469) | 30.39 (4.945) |
| | from baseline (SE) | | |
| Month 12 | n | 17 | 9 |
| | Mean | 50.13 | 37.75 |
| | SD | 18.082 | 26.048 |
| | Median | 53.36 | 41.01 |
| | Min, Max | 24.51, 93.32 | 0.00, 68.75 |
| | LS Means difference | 41.23 (3.506) | 28.66 (4.945) |
| | from baseline (SE) | | |

Results are summarized in Table 8.

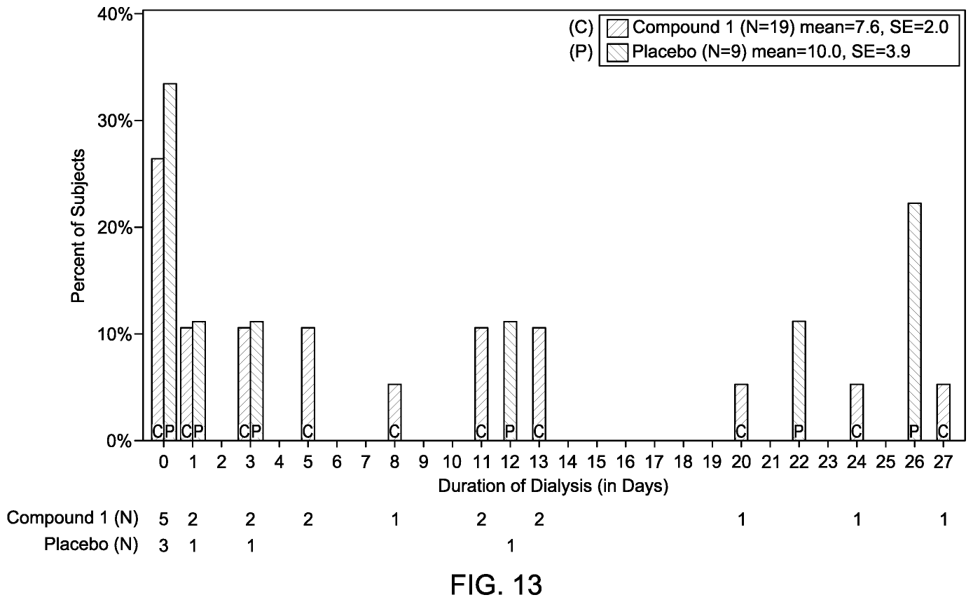

Duration of Dialysis: The duration of dialysis through Day 28 by treatment arm is shown in FIG. 13. As shown, on average duration of dialysis was 2.4 days shorter for the Compound 1 arm (mean=7.6, SE=2.0) vs placebo (mean=10.0, SE=3.9).

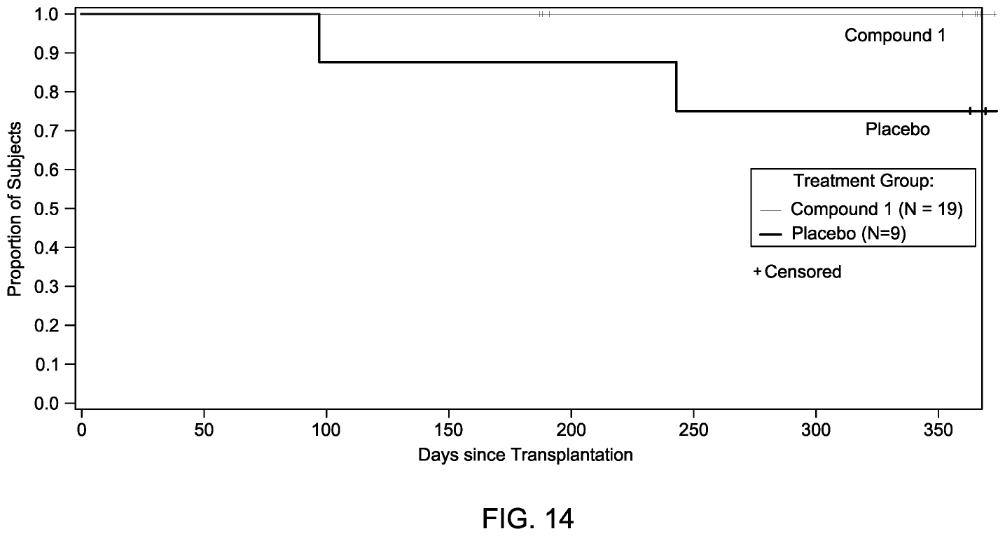

Graft Failure at 6 and 12 Months: FIG. 14 shows incidence of graft failure over time by study arm. Two subjects (22%) in the placebo arm experienced graft failure within the first 12 months after transplant vs no subjects in the Compound 1 arm (χ=4.66, P=0.03). Because there were no events in the Compound 1 arm, a hazard ratio could not be calculated. There was a discrepancy in the date of graft failure for one subject. At the 6-month visit, the subject reported that graft failure had already occurred and eGFR was not reported for this subject at 6 months. However, the investigator noted graft failure as an AE at 8 months, when the subject was hospitalized. For the purpose of this analysis, the more conservative 8-month hospitalization date reported by the investigator was used.

Adverse Events (AE): There were no deaths nor discontinuations of study drug due to adverse events. While the percent of subjects experiencing an AE was nearly equivalent across study arms, the number of AEs per subject was twice as high in the placebo arm than in the Compound 1 arm. In the Compound 1 arm, there was a total of 99 AEs reported in 17 (89.5%) subjects, an average of 5.8 events per subject. In the placebo arm, there was a total of 89 AEs reported in 8 (88.9%) subjects, an average of 11.1 events per subject.

Treatment-emergent adverse events (TEAEs) followed a similar pattern: while a slightly lower percent of subjects in the Compound 1 group experienced a TEAE, the number of events per subject was twice as high in the placebo group. In the Compound 1 arm, 83 TEAEs were reported in 15 (78.9%) subjects, an average of 5.5 TEAEs per subject (Table 9). Six of these TEAEs were assessed by the investigator as related to study drug. In the placebo arm, 78 TEAEs were reported in 8 (88.9%) subjects, an average of 9.8 TEAEs per subject (Table 9). None of the TEAEs in the placebo arm were assessed by the investigator as related to study drug. The majority of TEAEs were rated as mild: 72.3% in the Compound 1 arm, 78.2% in the placebo arm. However, there were 11 TEAEs in the Compound 1 arm rated as severe, vs 1 in the placebo arm. In 7 of the 12 system organ classes (SOCs) with TEAEs in >5% of subjects, the event rate in the placebo arm exceeded that in the Compound 1 arm by >10%. This was largely driven by higher dyspnoea and oedema in the placebo arm. In the one SOC in which the event rate in the Compound 1 arm exceeded the placebo arm (renal and urinary disorders), the difference was due to higher nocturia in the Compound 1 arm.

Serious adverse events (SAEs) followed a similar pattern (note: all SAEs were treatment emergent; Table 9). In the Compound 1 arm, 8 (42.1%) subjects reported a total of 16 TESAEs, an average of 2.0 TESAEs per subject. In the placebo arm, 4 (44.4%) subjects reported 17 TESAEs, an average of 4.3 TESAEs per subject. None of the TESAEs in either study arm were assessed by the investigator as related to study drug. The most common SOC for TESAEs was renal and urinary disorders, which included 2 incidents of acute renal failure in the placebo group. There was a pronounced difference in when TESAEs occurred. In the Compound 1 arm, 75% of TESAEs occurred ≤28 days after treatment. In the placebo arm, 76% of TESAEs occurred >28 days after treatment.

An overview of all AEs is shown in Table 9:

TABLE 9
Adverse Events-Overview

| | Compound 1 | | | Placebo | | | Total | | |
|---|---|---|---|---|---|---|---|---|---|
| | (N = 19) | | | (N = 9) | | | (N = 28) | | |
| Subjects with | n (%) | | Events | n (%) | | Events | n (%) | | Events |
| At least one AE | 17 | (89.5) | 99 | 8 | (88.9) | 89 | 25 | (89.3) | 188 |
| At least one TEAE¹ | 15 | (78.9) | 83 | 8 | (88.9) | 78 | 23 | (82.1) | 161 |
| TEAEs related to IMP² | 3 | (15.8) | 6 | 0 | | 0 | 3 | (10.7) | 6 |
| At least one severe TEAE | 6 | (31.6) | 11 | 1 | (11.1) | 1 | 7 | (25.0) | 12 |
| At least one SAE | 8 | (42.1) | 16 | 4 | (44.4) | 17 | 12 | (42.9) | 33 |
| At least one TESAE | 8 | (42.1) | 16 | 4 | (44.4) | 17 | 12 | (42.9) | 33 |

| | | | | | | |
|---|---|---|---|---|---|---|
| with TESAEs related to IMP | 0 | 0 | 0 | 0 | 0 | 0 |
| Deaths-All Causes | 0 | 0 | 0 | 0 | 0 | 0 |

Abbreviations: AE = adverse event; IMP = investigational medicinal product; SAE = serious adverse event; TEAE = treatment-emergent adverse event; TESAE = treatment-emergent serious adverse event.
Percentages are based on N.
¹An AE was considered treatment-emergent if the date of onset was on or after the date of the first dose of study treatment through 30 days after the last dose of study treatment, or, if applicable, those with onset prior to the first administration of study medication but that worsened while on therapy.
²Related = possibly related, probably related, or definitely related to IMP (study drug).

### Efficacy Conclusions

The totality of the efficacy data indicated that Compound 1 improves short-term and long-term renal function in patients who have undergone kidney transplantation and shown signs and symptoms of significant renal injury immediately post-transplantation. In the short-term, initial oliguria (<50 cc urine per hour for 8 consecutive hours post-transplant) was more likely to resolve in the Compound 1 arm in the 28 days post-transplant (Compound 1=78.9%; Placebo=44.4%), and resolved more quickly (Compound 1=5 days; placebo=14 days). Consistent with this result, over the first 14 days after transplant, daily urine output was higher, and increased more over baseline, in the Compound 1 arm.

Serologic results also showed a short-term efficacy signal in favor of Compound 1. While SCr was higher in the Compound 1 arm at baseline and Day 3, it was lower than placebo from on Days 7, 14 and 28. Adjusting SCr by gender, race and age to derive estimated glomerular filtration rate, the Compound 1 arm showed better eGFR at Days 14 and 28. CRP, a measure of inflammation, decreased in both study arms, but was higher in the Compound 1 arm at baseline and lower in the Compound 1 arm at Day 3 (68.6% reduction for Compound 1; 17.5% reduction for placebo). NGAL, a measure of kidney tissue damage, was higher in the Compound 1 arm at baseline, then decreased to a level similar to placebo at Day 3 (43.5% reduction). There was little change in the placebo arm (−12.5%).

There were also differences in clinical treatment indicating in favor of Compound 1. While the percentage of patients requiring dialysis in the first week, and number of dialysis sessions in the first 28 days did not differ between groups, duration of dialysis was 2.2 days shorter in the Compound 1 arm. Hospitalization for transplant was 3.8 days shorter in the Compound 1 arm.

There was a signal in favor of Compound 1 on both long-term outcome measures. eGFR was 11 to 13 mL/min/1.73 m² higher in the Compound 1 arm at 6 and 12 months. At one year, there were more graft failures in the placebo arm (n=2; 22%) than the Compound 1 arm (n=0; 0%).

Overall, urologic, serologic and clinical treatment measures manifested a pattern in which patients in the Compound 1 arm showed improved kidney function when compared to placebo.

### Example 2. Synthesis of Compound 1

As described in Example 7 of WO 2004/058721, Compound 1 was synthesized according to Scheme 1.

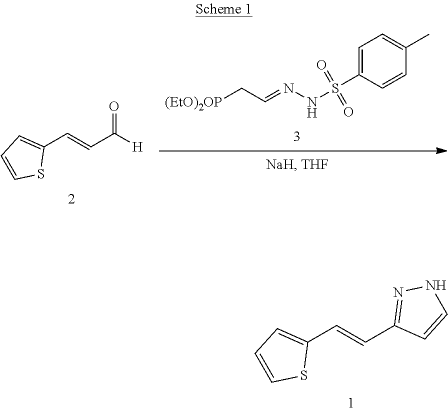

To a solution of diethoxyphosphorylacetaldehyde tosylhydrazone (3, 75 g) in 400 mL of THE was added 11.6 g of 60% NaH in portions, and the solution was stirred for 15 min. The solution was cooled to 0° C., and then a solution of 3-(2-thienyl)acrylaldehyde (2) in 100 mL THE was added dropwise. The reaction was then stirred at room temperature for 1 hour, then at reflux for 1 hour. The reaction mixture was partitioned between 5% NaH₂PO₄ and ethyl acetate. The organic layer was separated, washed with water and brine, dried over magnesium sulfate, filtered and concentrated to provide the crude product as a brown oil. Purification via silica gel column chromatography afforded 8.3 g of a yellow powder. Trituration with dichloromethane/hexane afforded 4.4 g of yellow powder having >98% purity: ¹H NMR (CDCl₃) δ 6.47 (d, 1H, J=1.5 Hz), 6.93 (d, 1H, J=9.9 Hz), 6.99 (dd, 1H, J=3.9, 2.1 Hz), 7.06 (d, 1H, J=2.1 Hz), 7.20 (d, 1H, J=3.9 Hz), 7.22 (d, 1H, J=9.9 Hz), 7.57 (d, 1H, J=1.5 Hz).

The conversion of 3-arylacrylaldehydes into substituted pyrazoles via treatment with diethoxyphosphorylacetaldehyde tosylhydrazone (3) is described in the literature (Almirante, N.; Cerri, A.; Fedrizzi, G.; Marazzi, G.; Santagostino, M. Tetrahedron Lett. 1998, 39, 3287). 3-(2-thienyl)acrylaldehyde (2) was prepared from 2-thienaldehyde and acetaldehyde as described in Heskin, H., Miller, R. E., Nord, F. F. J. Org. Chem. 1951, 16, 199.

### Example 3. Mean eGFR Results Suggest Increased Life Expectancy in Subjects Receiving Compound 1

The National Kidney Foundation has set guidelines for classification of chronic kidney disease (CKD) based in part on a subject's eGFR and summarized in Table 10 below (Levey, A. S., et al. National Kidney Foundation Practice Guidelines for Chronic Kidney Disease: Evaluation, Classification, and Stratification. Ann. Intern. Med. 2003; 139:137-147; UK and Scottish Renal Associations. Consensus statement on management of early CKD, February 2007. Edinburgh: Royal College of Physicians of Edinburgh; 2007).

TABLE 10

| | eGFR (mL/ | |
|---|---|---|
| Stage | min/1.73 m²) | Description |
| 1 | ≥90 | Kidney damage with normal or increase GFR |
| 2 | 60-89 | Kidney damage with mild decreased GFR |
| 3A | 45-59 | Moderately decreased GFR |
| 3B | 30-44 | |
| 4 | 15-29 | Severely decreased GFR |
| 5 | <15 | Kidney failure |

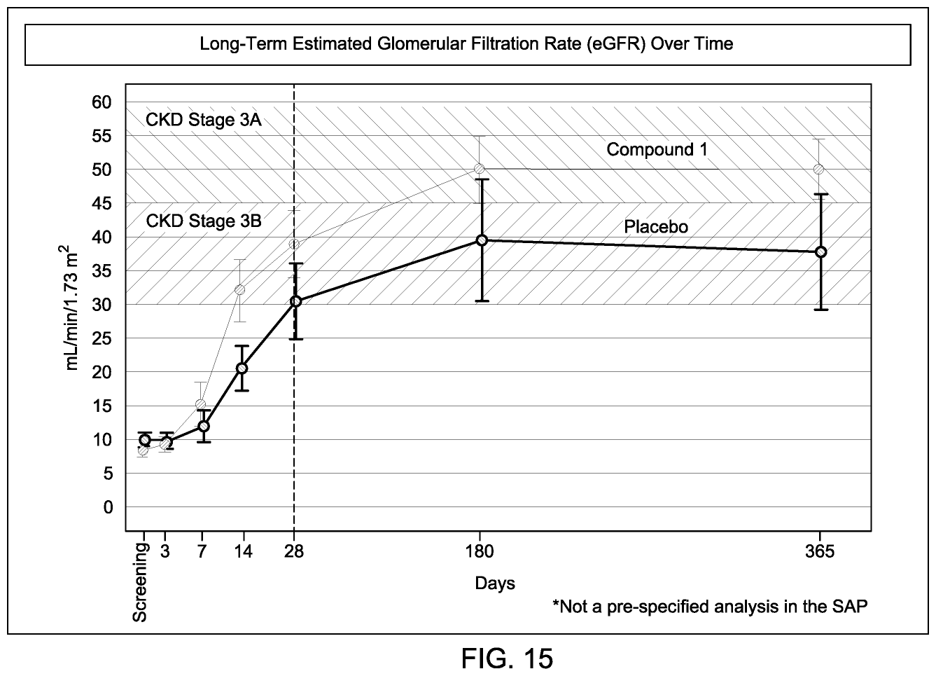

FIG. 15 shows mean eGFR (mL/min/1.73 m²) by study arm at screening, Day 3, Day 7, Day 14, Day 28, Month 6 and Month 12, overlaid with the National Kidney Foundation's predictive chronic kidney disease staging criteria for eGFR. As shown in FIG. 15, subjects who received Compound 1 reached one stage lower than those receiving a placebo after 6 months and after 12 months, indicating better outcomes for these subjects.

Evidence suggests that subjects at lower stages on the CKD scale have significant long-term mortality benefits versus patients at higher stages. For example, an average 7.7 year increase in life expectancy, on average, is projected for a 50-year-old male moving from CKD Stage 3B disease down to CKD Stage 3A disease. Similarly, a 9.1 year increase in life expectancy, on average, is projected for a 50-year-old female moving from CKD Stage 3B disease to CKD Stage 3A disease. (Turin, T. C., et al. Chronic kidney disease and life expectancy. Nephrol. Dial. Transplant. 2012; 27:3182-3186.) Thus, administration of Compound 1 to subjects at risk of DGF, as provided herein, is expected to increase life expectancies of those subjects.

### Example 4. A Multicenter, Prospective, Double-Blind, Randomized, Placebo-Controlled, Phase 3 Study of Compound 1 to Improve Graft Function and Reduce the Severity of Kidney Dysfunction or Delayed Graft Function in Recipients of a Deceased Donor Kidney

### Objectives

Primary: To improve graft function and reduce the severity of kidney dysfunction or delayed graft function following kidney transplantation in recipients of a deceased donor kidney.

Secondary:
To demonstrate the safety and efficacy of Compound 1 in recipients of a deceased donor renal allograft with high risk of DGF.
To demonstrate the efficacy of Compound 1 in reducing the intensive requirement for healthcare resource utilization in recipients of a deceased donor renal allograft with DGF.

### Criteria for Evaluation

### Efficacy:

Primary Endpoint: The primary endpoint is renal function assessed by eGFR (using the CKD-EPI equation based on serum creatinine), with a primary analysis time point consisting of eGFR at month 12.

Secondary Endpoint:
The proportion of patients with eGFR>30 at Day 30, Day 90, Day 180, and Day 360 (at each time point).
The proportion of subjects categorized as experiencing (1) Primary Non Function (PNF); (2) DGF (dialysis within the first 7 days); (3) slow graft function (SGF; defined as sCr>3 mg/dL within the first 7 days, without dialysis required); or (4) none of the above.
Length of hospitalization following transplantation.
The severity of DGF assessed by its duration of dialysis as measured by the number of days a patient remains dialysis dependent from the first day of treatment (Day 1) until the day of the last dialysis session, which is followed by 7 consecutive dialysis-free days, or until Day 30, whichever comes first.

For the severity of dialysis secondary endpoint (fourth endpoint above), the last dialysis session is defined as the day when a patient receives his/her last dialysis before achieving 7 consecutive dialysis-free days, or by Day 30, whichever comes first. Patients who stop dialysis in the period from Day 24 to Day 29 are followed for 7 days, even though that requires following the patient beyond Day 30, to be sure they have met the requirement for 7 consecutive dialysis-free days. For example, patients whose last dialysis session is on Day 29, must remain dialysis-free to Day 36 in order for Day 29 to be considered the last day of dialysis dependency. The maximal day of the assessment for the duration of dialysis endpoint is 36 days. If dialysis is reinstituted after a dialysis-free period of at least 7 days, but still within the first 30 days post-transplant, it is recorded, but is considered to be a new episode of renal insufficiency and will not be considered to be DGF. If a patient or graft is lost within the first 30 days, it is imputed that the patient required dialysis until Day 30 (i.e., the worst result with respect to the duration of dialysis endpoint). Primary non-function (PNF) is defined as continuous dialysis dependency for at least 60 days post-transplant. Such patients are also counted as having required dialysis out to 30 days, but the incidence of PNF will also be recorded.

Exploratory Endpoints:
Incidence of DGF assessed by proportion of patients requiring dialysis within the first 7 days post-transplant.
The proportion of patients with PNF, defined as a continuous requirement for dialysis for at least 60 days post-treatment.
Proportion of patients with acute rejection.
Plasma and urine biomarkers such as: C-reactive protein (CRP), neutrophil gelatinase-associated lipocalin (NGAL), and kidney injury molecule-1 (KIM-1).

Safety:

Safety will be assessed by the following parameters:
Adverse events (AEs)
Treatment-emergent AEs
Serious AEs (SAEs)
Physical examination
Vital signs
Laboratory tests (clinical blood chemistry, coagulation, hematology, urinalysis)
Electrocardiogram
Pregnancy test
Concomitant medications/treatments

### Methodology

The study is a randomized, placebo-controlled, multicenter, double-blind, Phase 3, two-arm parallel study. The Sponsor and all site personnel, including site monitors, patients, the Medical Monitor, and the Investigators are blinded to individual treatment assignments for the duration of the study.

Patients in this study are at enhanced risk for ischemia-reperfusion injury leading to early poor function or the need for dialysis in the first week following transplantation of a kidney from a deceased donor. Patients who fulfill all other eligibility criteria are randomized in a 1:1 fashion to receive 2 mg/kg Compound 1 or placebo (normal saline). Study drug is administered daily over a 30-minute period (±5 minutes) for a total of 3 intravenous (IV) infusions.

Recipients of a kidney from either a donation after brain death (DBD) donor or donation after cardiac death (DCD) donor are considered eligible and are stratified at randomization. Recipients of DCD kidneys are capped at 20% of enrollment. Kidneys may be preserved by static cold storage or cold pulsatile machine perfusion (PMP), but PMP kidneys are capped at 40% of enrollment. Recipients of normothermic pulsatile machine perfused kidneys, which is still an emerging technology, may not be enrolled in this study. These randomization stratification factors will be included in the primary efficacy analysis.

The primary efficacy outcome is the assessment of renal function by eGFR (as estimated using the CKD-EPI sCr based equation).

All patients enrolled are followed for the assessment of the primary efficacy variable and out to Day 360 for the specified safety and renal function assessments. All adverse events are collected until Day 90. Thereafter, serious adverse events and events including rejection episodes including BPACR [biopsy proven acute cellular rejection] and tACR [treated biopsy proven or clinically suspected acute cellular rejection] and antibody-mediated rejection [AMR], as well as malignancies and clinically significant infections are continued to be collected out to Day 360. Visits past Day 180 collect information either via phone or medical chart/record review. Renal function (SCr, eGFR) and patient and graft survival are also collected out to Day 360. In addition, the calcineurin (CNI) dose and the associated trough level (also collected as standard of care) are collected through Day 360.

### Study Population

The study will be conducted in recipients of a deceased donor renal allograft who are at high risk for ischemia-reperfusion injury leading to early poor function or the need for dialysis in the first week following transplantation, as delineated below.

Inclusion Criteria:
All patients must provide written informed consent using an Institutional Review Board/Independent Ethics Committee approved consent form and must understand and be willing and able to comply with the requirements of the study, including screening procedures and all required study visits.
Males and females greater than or equal to 18 years of age.
Renal failure requiring hemodialysis or peritoneal dialysis initiated at least 3 months prior to transplantation.
Patient is to be the recipient of a first kidney transplant from a deceased donor.
Study drug can be administered starting within 30 hours after restoration of blood flow to the engrafted kidney.
Body mass index <40 based on patient's body weight. Body weight and height parameters obtained within 7 days prior to study entry may be used.
Estimated donor organ cold ischemia time <30 hours (for PMP kidneys <40 hours).
Females of childbearing potential (including perimenopausal women who have had a menstrual period within 1 year):
   Must have a negative serum pregnancy test prior to transplantation.
   Must agree to use 2 forms of effective birth control regimen (at least one-barrier method) during the initial 30-day study period or agree to maintain total abstinence throughout the initial 30-day study period.
Male patients must agree to use condoms or other suitable means of pregnancy prevention such as abstinence during the initial 30-day study period.
Patient has poor renal function in the first 24 hours post-transplantation based on an average urine output (UO) of <50 mL/hour over any 8 consecutive hours, to maximize the likelihood that the patient requires dialysis within the first 7 days post-transplant, irrespective of pre-transplant donor and recipient risk factors.
Reason for low UO is not due to structure or vascular abnormalities which, when indicated should be confirmed with a renal ultrasound with Doppler study and/or vascular or urinary tract contrast studies.

Exclusion Criteria:
Scheduled for multiple organ transplantation or prior recipient of a transplanted organ.
Recipient of an ABO-incompatible kidney.
Recipient of pediatric en bloc kidney transplantation or adult or pediatric planned transplant of dual kidneys (from the same donor) not transplanted en bloc.
Recipient of a kidney preserved by normothermic PMP.
Has measurable donor-specific antibody or positive cross-match requiring desensitization prior to transplantation or deviation from standard immunosuppressive therapy.
Either the donor or the recipient is currently participating in or has participated in an investigational drug or medical device study within 30 days or five drug half-lives, whichever is longer, prior to enrollment into this study. Patients cannot be given another investigational agent during the course of this study (through Day 360). Patients (recipient and donor kidneys) may participate in another concurrent study only if that study is a non-interventional, observational investigation.
Concurrent sepsis or active bacterial infection.
Has an active malignancy or history within 5 years prior to enrollment in the study of solid, metastatic or hematologic malignancy with the exception of basal or squamous cell carcinoma in situ of the skin that has been adequately treated.
Female who is breastfeeding.
History of positive human immunodeficiency virus test.
Requires treatment with the cytochrome P450 (CYP) 1A2 inhibitors, ciprofloxacin and/or fluvoxamine (Luvox®).
Unwilling or unable to comply with the protocol or to cooperate fully with the Investigator or the site personnel.
Not deemed medically appropriate for the study in the opinion of the Investigator.

### Investigational Product, Dosage and Mode of Administration

A Compound 1 stock solution (10 mg/mL) is diluted at the clinical site. Normal saline is used as placebo. Eligible patients receive placebo or 2 mg/kg of Compound 1, via IV infusion over 30 minutes, once daily for 3 days, with the first dose administered within 30 hours after transplantation in patients who meet inclusion criteria for treatment of DGF. The subsequent two doses are administered 24±2 hours after the previous dose.

Patients are randomized to Compound 1 or placebo in a 1:1 ratio using a central randomization process. Allocation to treatment is stratified on type of donor: DBD or DCD, and on method of preservation (cold static storage or cold machine perfusion). No more than 20% of the enrolled patients will have kidneys from DCD donors and no more than 40% of the transplanted kidneys will have been preserved by cold PMP.

### Duration of Treatment

2 mg/kg of Compound 1 or placebo by IV infusion once daily for 3 days.

### Duration of Study

The planned duration of this study is 360 days.

### Reference Therapy, Dosage and Mode of Administration

Placebo (normal saline) IV infusion.

### Concomitant Medications

Induction and maintenance immunosuppression and concomitant medications are administered to all patients according to standard institutional protocol. If approved generic substitutions are available for an agent, they may be used, but the same generic alternative is used for a patient throughout the study until at least Day 90.

All concomitant medications are recorded from the time of study enrollment to Day 90. Thereafter, immunosuppressants, antimicrobials, angiotensin converting enzyme (ACE) inhibitor and angiotensin-receptor blocker (ARBs) usage are recorded through Day 360. Medications specific to the administration of anesthesia for surgery are not recorded.

All patients receive antibody induction therapy, preferably with rabbit polyclonal anti-thymocyte globulin but anti-CD25 monoclonal antibodies are also allowed (anti-IL2R, basiliximab). Thymoglobulin is administered at a dose of 1.5 mg/kg administered over 4 to 6 days as tolerated, preferably with the first dose administered in the operating room. Adjustments in the dose and/or frequency of delivery is permitted to allow for institutional standard of care dosing, however the reasons should be clearly stated in the medical record and appropriate case report form. Alternatively, basiliximab is administered in compliance with the product labeling.

Initial maintenance immunosuppression is tacrolimus in combination with either mycophenolate mofetil (MMF) or mycophenolate sodium (MPS). Tacrolimus is started within 48 hours of transplantation; if a delay is needed for cause, the Medical Monitor should be consulted. Cyclosporine may be used in place of tacrolimus if approved by the medical monitor. Switching from tacrolimus to cyclosporine is allowed for clinical cause.

MMF is administered at 750-1000 mg twice a day. Corresponding doses of MPS as recommended in the package insert are used. In all cases, the dose may be adjusted for clinical cause. Azathioprine may be substituted for mycophenolic acid-based therapy if necessary after consultation with the Medical Monitor. Initial use of belatacept, sirolimus or everolimus is not allowed but if switching of immunosuppression is needed for cause, the medical monitor should be consulted and the reason for the switch should be recorded.

All subjects receive concomitant corticosteroids with a rapid taper from high dose to a maintenance dose of at least 5 mg per day to be maintained until at least Day 30. Full steroid withdrawal is permitted only after 30 days post-transplant. Complete steroid avoidance or immediate minimization is not allowed. Daily dose levels of corticosteroids are recorded. Adjustments in the dose is permitted to allow institutional standard of care dosing, however the reasons should be clearly stated in the medical record and appropriate case report form.

Anti-viral, anti-fungal and anti-microbial prophylaxis is required but is to be based on local standard of care, including regimens for cytomegalovirus and Pneumocystis jiroveci and other antiviral, antifungal, and antimicrobial prophylaxis.

### Statistical Methods

All statistical tests are two-sided and at the 5% level of significance.

### Efficacy Analysis

The primary analysis of the primary and secondary efficacy endpoints are based on the Full Analysis Set (FAS), where all subjects who are randomized (and treated) and who received a deceased donor renal allograft are analyzed. The primary efficacy endpoint is eGFR assessed by the (sCr based) CKD-EPI equation, with a primary time point of eGFR at 12 months estimated using a Mixed Model Repeated Measures (MMRM) approach, adjusting for stratification factors. Analyses for continuous secondary and exploratory endpoints are assessed similarly, except where endpoints do not represent repeated assessments (e.g. duration of dialysis and length of hospitalization) which are assessed in an ANOVA framework.

Analysis of the proportion of subjects with eGFR>30 (over time) is conducted using a Generalized Linear Model (GLM), adjusting for stratification factors.

The difference between treatments with respect to categorical endpoints such as the incidence of Delayed Graft Function (DGF) and Primary Non Function (PNF) and separately for the ordinal categories (the percentage of subjects experiencing 1) PNF, 2) DGF (dialysis within the first 7 days) or 3) SGF (defined as sCr>3 mg/dL within the first 7 days, without dialysis required) or 4) none of the above) are analyzed using Cochran-Mantel-Haenszel (CMH) test stratified by the randomization stratification variables. For the ordinal categories of PNF, DGF, SGF or none, the test of interest is the test of differences in row mean scores.

### Safety Analysis

All subjects receiving any part of at least one infusion of study treatment are evaluated for safety. The safety analyses include evaluation of the incidence of treatment-emergent AEs, Grade 3 or greater AEs, SAEs, and AEs leading to discontinuation of study treatment. Laboratory and vital signs assessments are evaluated over time on study using descriptive statistics. Shift analyses of relevant clinical laboratory parameters are produced showing shifts across low, normal, and high categories.

Incidence rates (e.g., BPACR, tACR, AMR, malignancies) are compared between treatment groups, as well as Graft and subject survival.

### Example 5. Effect of Compound 1 on Growth of c-Met-expressing Human Tumor Cells in Immunocompromised Mice

The effect of Compound 1 on growth of c-Met-expressing human tumor cells was evaluated in immunocompromised mice using xenograft models of pancreatic, colon, and brain cancer. As detailed below, Compound 1 treatment did not adversely affect survival in an in vivo model of c-Met-expressing glioma in immunocompromised mice, nor did it promote tumor growth in in vivo models of c-Met-expressing colon cancer and pancreatic cancer in immunocompromised mice. Thus, exposure to Compound 1 was not associated with increases in tumor size or tumor weight, nor was it associated with a decrease in survival compared to vehicle-treated animals.

### Glioma Orthograft Model

Human glioma cells U87-MG (obtained from American Type Culture Collection [ATCC]), which express c-Met (La, B., et al. Clin. Cancer Res. 2005 Jun. 15; 11(12):4479-86), were used in the glioma orthotopic model. U87-MG cells were maintained and cultured using protocols provided by ATCC and summarized as follows. Cells were grown in culture flasks (T75 and T150) and incubated at 37° C. in an incubator conditioned with 5% C02. The culture medium used was Eagle's minimum essential medium (EMEM) modified with Eagle's balanced salt solution and 2 mM L-glutamine, 1 mM sodium pyruvate, 0.1 mM nonessential amino acids, and 1.5 g/L sodium bicarbonate, supplemented with 10% fetal bovine serum (FBS). To harvest cells, growth medium was removed and cell monolayer was rinsed with phosphate buffered saline (PBS) and treated with trypsin-ethylenediaminetetraacetic acid (EDTA) solution (0.25% w/v trypsin, 0.03% w/v EDTA) until the cell monolayer was detached as monitored under a microscope. Cells were separated from each other by gentle pipetting. Cells in culture were split twice a week at a ratio of 1:3 to 1:5. Harvested cells were suspended in EMEM medium for animal injection. Cells (2×105 cells in 10 μL) were injected intracranially into BALB/c nude mice. Tumors were allowed to grow for 7 days, then animals were treated daily until day 28 with 2 mg/kg Compound 1 or vehicle administered via 50 μL IP injection. Survival of the mice was monitored daily until all mice died.

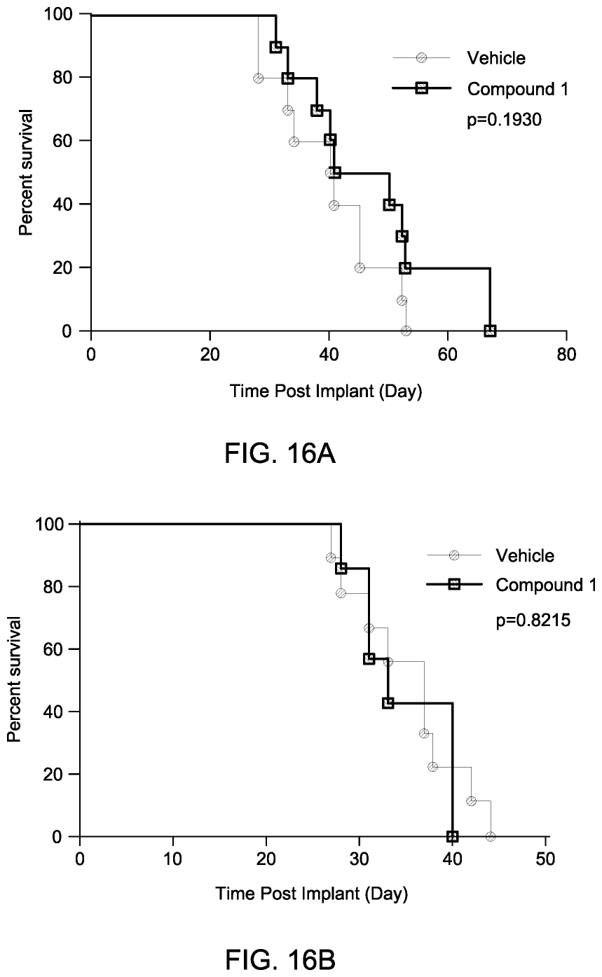

Compound 1 was evaluated in two independent studies in the orthotopic glioma model described above, and the results are summarized in FIG. 16A and FIG. 16B. Compound 1 treatment did not decrease survival rate, as compared with a vehicle control group for each of the two studies (log-rank test, A and B, p=0.1930 and p=0.8215, respectively).

### Colon Tumor Xenograft Model

Human colon cancer cells HT29 (obtained from ATCC), which express c-Met (Wielenga, V. J., et al. Am. J. Pathol. 2000 November; 157(5):1563-73), were used in the colon tumor xenograft model. HT-29 cells were maintained and cultured using protocols provided by ATCC and summarized as follows. Cells were grown in culture flasks (T75 and T150) and incubated at 37° C. in an incubator conditioned with 5% CO2. The culture medium used was McCoy's 5A medium modified to contain 1.5 mM L-glutamine and 2200 mg/L sodium bicarbonate, supplemented with 10% FBS. To harvest cells, growth medium was removed and the cell monolayer was rinsed with PBS and treated with trypsin-EDTA solution (0.25% w/v trypsin, 0.03% w/v EDTA) until the cell monolayer was detached as monitored under a microscope. Cells were separated from each other by gentle pipetting. Cells in culture were split twice a week at a ratio of approximately 1:5. Harvested cells were suspended in growth medium containing no serum for animal injection. Cells (1×106 cells in 100 μL) were injected SC in the right hind flank of BALB/c nude mice. Tumors were allowed to grow for 7 days, then animals were treated daily until Day 27 with 2 mg/kg Compound 1 or vehicle administered via 50 μL IP injection. Tumor size was measured with dial calipers on Days 7, 12, 15, 19, 22, 27 and survival was recorded. Animals were sacrificed on Day 27 and tumor size and weight was recorded.

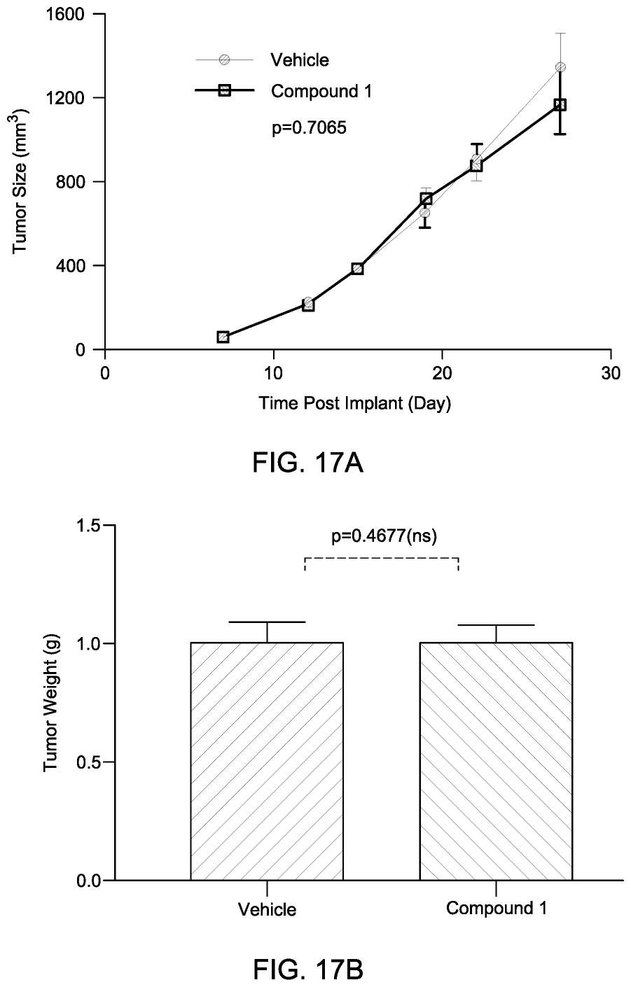

Compound 1 was evaluated in a human colon tumor xenograft model described above, and the results are summarized in FIG. 17A and FIG. 17B. Compound 1 treatment did not increase the size (FIG. 17A) or weight (FIG. 17B) of the colon tumor xenograft (p=0.7065 [ANOVA] and p=0.4677 [T-test], respectively).

### Pancreatic Tumor Xenograft Model

Human pancreatic ductal carcinoma cells SUIT-2 (obtained from the Japanese Collection of Research Bioresources [JCRB] Cell Bank), which express c-Met (Maehara, N., et al. ChBr J. Cancer 2001 Mar. 23; 84(6):864-73), were used in the pancreatic tumor xenograft model. SUIT-2 cells were maintained and cultured using protocols provided by the JCRB Cell Bank and summarized as follows. Cells were grown in culture flasks (T75 and T150) and incubated at 37° C. in an incubator conditioned with 5% C02. The culture medium used was Roswell Park Memorial Institute (RPMI) with 10% FBS. To harvest cells, growth medium was removed and the cell monolayer was rinsed with PBS and treated with 0.25% trypsin in PBS until the cell monolayer was detached as monitored under a microscope. Cells were separated from each other by gentle pipetting. Cells in culture were split weekly. Harvested cells were suspended in growth medium containing no serum for animal injection. Cells (5×106 cells in 100 μL) were injected SC in the right hind flank of male BALB/c nude mice. Tumors were allowed to grow for 12 days, then animals were treated daily, 5 days a week, for 3 weeks with 2 mg/kg Compound 1 or vehicle administered via 50 μL IP injection. Tumor volume was measured twice weekly using dial calipers, and tumor weight was measured at sacrifice. Animals were sacrificed after the last dose administration.

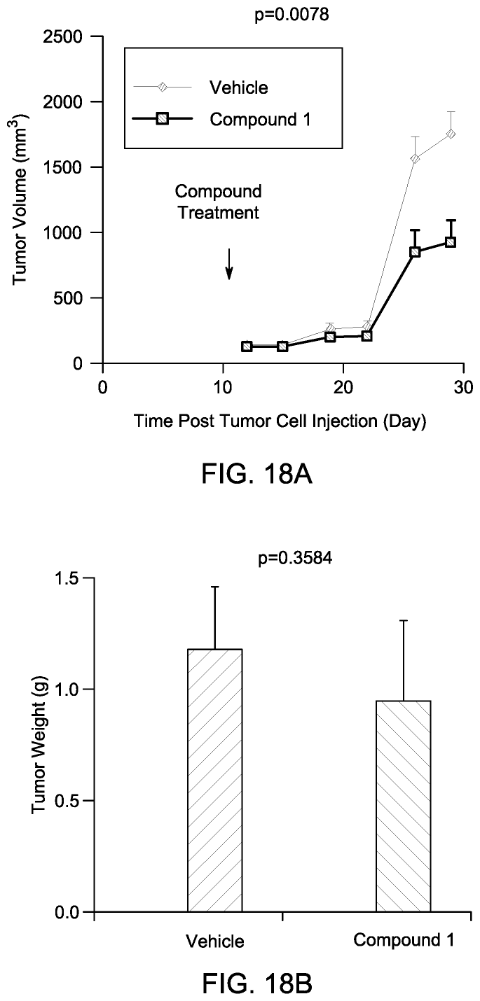

Compound 1 was evaluated in a human pancreatic tumor xenograft model, and the results are summarized in FIG. 18A and FIG. 18B. Compound 1 treatment did not increase the size (FIG. 18A) or weight (FIG. 18B) of the pancreatic tumor. In fact, Compound 1 reduced the size of the tumor (p=0.0078, ANOVA), although it did not significantly reduce the weight of the tumor (p=0.3584, T-test).

### Example 6. Alternative Synthesis of Compound 1

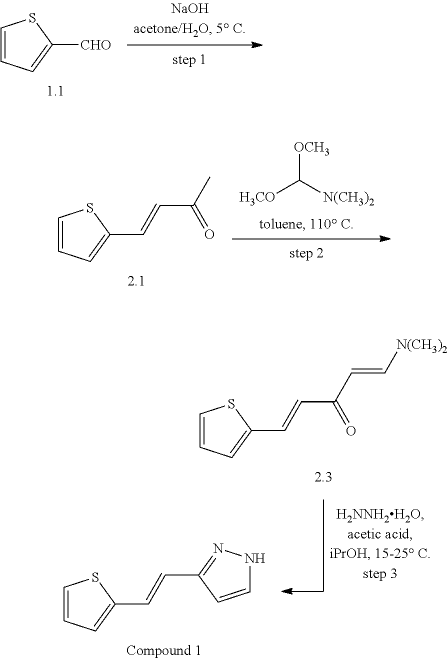

### Step 1. Synthesis of (E)-4-(thiophen-2-yl)but-3-en-2-one (2.1)

To a solution of thiophene-2-carboxaldehyde (1.1, 40 kg) in 70 L of acetone was added 80 kg of 0.4 M aqueous NaOH slowly at 5° C. The solution was warmed to room temperature and stirred for 2-3 hours. Dichloromethane (approx. 4.5 volumes) was then added to the reaction mixture, the layers were allowed to separate, and the organic layer was removed. The aqueous layer was extracted with dichloromethane (approx. 0.7 volumes), the layers were allowed to separate, and the organic layer was combined with the first organic layer. Water (approx. 0.8 volumes) was added, the layers were allowed to separate, and the organic layer was combined with the first and second organic layers. The combined organic layers were then concentrated and diluted with toluene, which was then distilled with a Dean Stark apparatus to remove water. The mixture was then polish filtered and further concentrated to 3-4 volumes to provide a solution of (E)-4-(thiophen-2-yl)but-3-en-2-one (2.1) in toluene, which was carried forward into the next step without further purification.

### Step 2. Synthesis of (1E,4E)-1-(dimethylamino)-5-(thiophen-2-yl)penta-1,4-dien-3-one (2.3)

To a solution of 2.1 in toluene from the previous step, N,N-dimethylformamide dimethylacetal (2.2a, 97 kg), was added and the reaction mixture heated at reflux for 36 hours. The reaction mixture was then concentrated to 2.5-3.5 volumes. The resulting slurry was cooled to room temperature and ethyl acetate was added. The resulting solids were filtered, washed with ethyl acetate, and dried under vacuum to afford (1E,4E)-1-(dimethylamino)-5-(thiophen-2-yl)penta-1,4-dien-3-one (2.3) in 55% yield from thiophene-2-carboxaldehyde (2.1).

In some instances, a slurry of seed material of 2.3 in ethyl acetate was added after concentration of the reaction mixture. Seed material of 2.3 can be prepared using the process described above.

### Step 3. Synthesis of Compound 1

2.3 (40 kg) was dissolved in 130 kg of isopropyl alcohol under nitrogen, and the mixture was cooled to 10° C. Acetic acid (13 kg) was then added at 5-25° C. The solution was then cooled to 5-15° C., followed by slow addition of hydrazine hydrate (11.5 kg) while maintaining a temperature of 10-25° C. The reaction mixture was then stirred at 20° C. Once the reaction was judged to be complete (e.g., using HPLC or thin layer chromatography), water was added, and the resulting solids were filtered, washed with a mixture of isopropyl alcohol and water (1:4), and dried to afford crude Compound 1 in 90% yield.

Crude Compound 1 (26 kg) was dissolved in 42 kg acetonitrile at 65-75° C. and then slowly cooled to 15-25° C. to induce crystallization. The resulting solids were filtered and washed with a mixture of acetonitrile and water, and then dried under vacuum. The solids were then dissolved in ethyl acetate at 55-65° C. and polish filtered. n-Heptane (200 kg) was then added to the mixture at 50-60° C. The mixture was cooled to 15-25° C., the resulting solids were isolated by filtration, washed with n-heptane, and dried under vacuum to afford crystalline Compound 1 in 67% yield.

### Example 7. Preparation and Characterization of Compound 1 Solid Form A

### General Methods:

### X-Ray Powder Diffraction (XRPD)

XRPD patterns of samples from scaled-up preparations were recorded at ambient temperature on a Bruker D8 Advance X-ray diffractometer (Karlsruhe, Germany) using Cu Kα radiation (λ=1.54 Å) at 40 kV, 40 mA passing through a Vario monochromator (Karlsruhe, Germany). The sample was loaded on a zero-background holder and gently pressed by a clean glass slide to ensure co-planarity of the powder surface with the surface of the holder. Data were collected in a continuous scan mode with a step size of 0.05° and dwell time of 1 s over an angular range of 3° to 400 2θ. Obtained diffractograms were analyzed with DIFFRAC.EVA diffraction software (Bruker, Wisconsin, USA).

In some cases, the X-ray intensity data were measured on a Bruker D8 Eco diffractometer system equipped with a graphite monochromator and a Cu Kα Sealed tube (λ=1.54 Å). The sample was loaded in a polyimide capillary and collected data in transmission mode. Bruker's APEX3 software suite (Bruker, Wisconsin, USA) was used to collect and extract the intensity data. Obtained diffractograms were analyzed with TOPAS software (Bruker, Wisconsin, USA). Mercury 4.2.0 software (Build 257471, Cambridge Crystallographic Data Centre, UK) was used to calculate the XRPD patterns from single crystal data.

### Thermogravimetric Analysis (TGA)

TGA was performed using a Discovery TGA 5500 (TA® Instruments, New Castle, Del., USA) instrument operating with TRIOS software (Version 5.0). The sample was placed in an aluminum pan. The sample cell was purged with dry nitrogen at a flow rate of 15 mL/min. A heating rate of 10° C./min from 25° C. to desired temperature was used in all the experiments.

### Differential Scanning Calorimetry (DSC)

Conventional DSC experiments were performed using a Discovery DSC 250 (TA® Instruments, New Castle, Del., USA) instrument equipped with a refrigerated cooling system (RCS90) and operating with TRIOS software (Version 5.0). The sample cell was purged with dry nitrogen at a flow rate of 50 mL/min. Accurately weighed samples (2-5 mg) placed in TZero pans with a pin hole were scanned at a heating rate of 10° C./min over a temperature range of 25° C. to desired temperature was used in all the experiments.

### Compound 1 Lot I

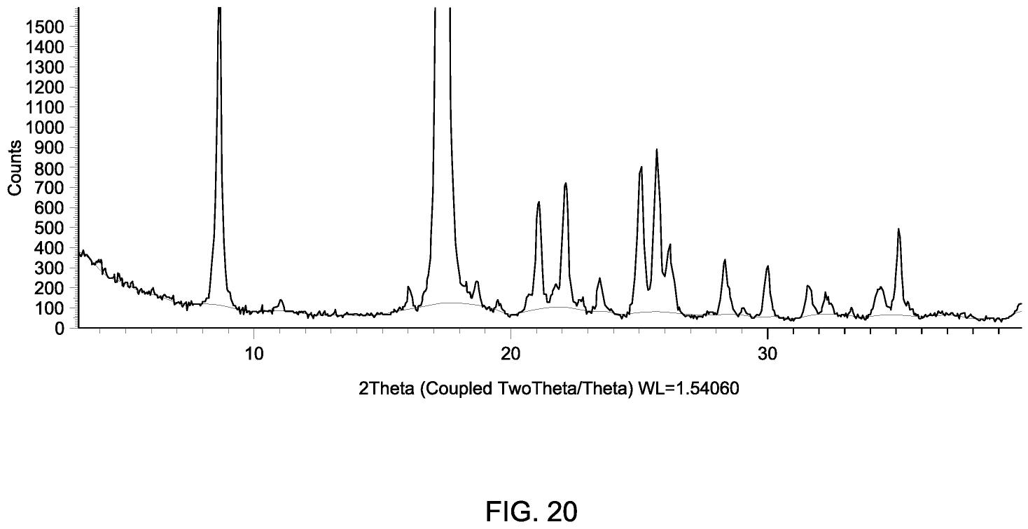

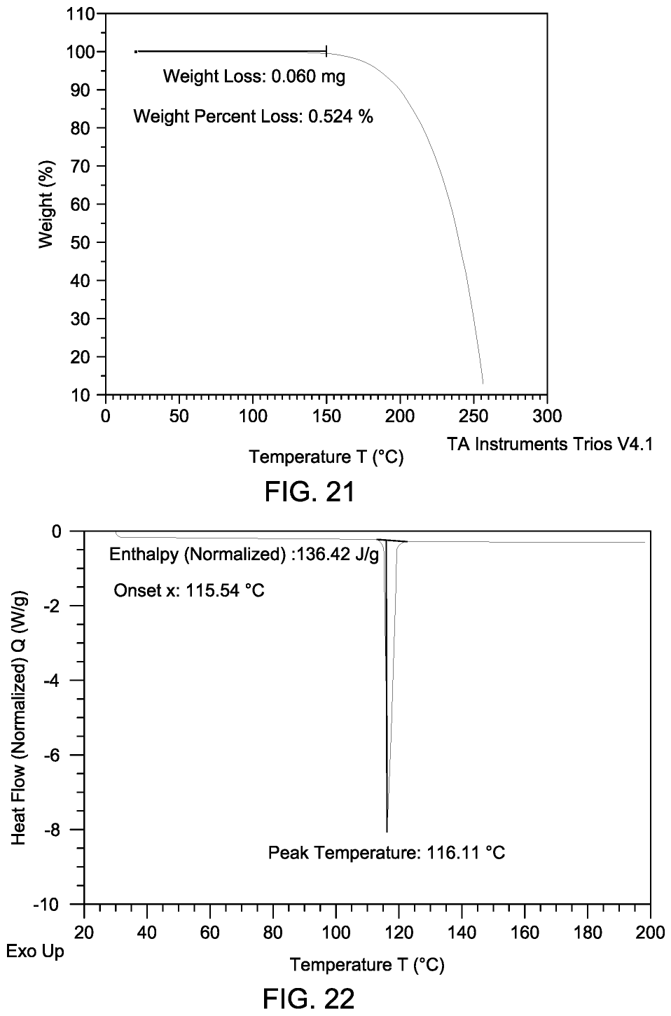

Compound 1 was provided (e.g., via the method of Example 6) in a form with an XRPD as shown in FIG. 20, a TGA as shown in FIG. 21, and a DSC as shown in FIG. 22. Herein, this material is referred to as “Terevalefim Lot I.”

### Form A

Compound 1 Form A was synthesized by recrystallizing Compound 1 Lot I from methanol. In a typical reaction, ˜450 mg of Compound 1 Lot I was dissolved in 2 mL of methanol while heating at 50° C. Resultant solution was kept at room temperature and allowed for slow evaporation of the solvent. Crystals suitable for single crystal X-ray diffraction were obtained within one day.

Compound 1 Form A bulk powder was prepared as follows: ˜5 g of Compound 1 Lot I was suspended in 5 mL of methanol and slurried at room temperature for two days. The resulting solid was filtered using 0.45 μm PTFE syringe filter.

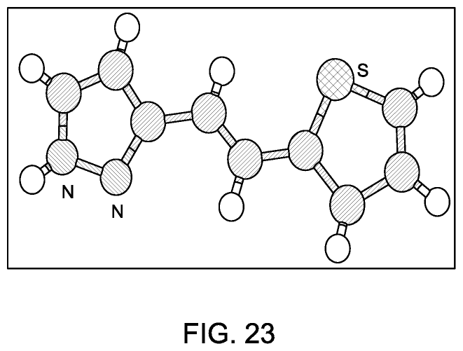

Single crystal X-ray diffraction of Compound 1 Form A was obtained (FIG. 23). Crystal data and structure refinement parameters are summarized below:

| | |
|---|---|
| Temperature (K) | 278.15 |
| Crystal system | Monoclinic |
| Space group | C2/c |
| a (Å) | 21.5572(10) |
| b (Å) | 4.9949(2) |
| c (Å) | 16.8890(8) |
| α (°) | 90 |
| β (°) | 108.445(2) |
| γ (°) | 90 |
| Volume (Å³) | 1725.12(14) |

The XRPD pattern of Compound 1 Form A calculated from single crystal X-ray diffraction data is shown in FIG. 24 and is summarized below:

| 2θ (°) | d-spacing (Å) | Intensity Counts (I) |
|---|---|---|
| 8.64 | 10.22 | 1690 |
| 11.04 | 8.01 | 422.5 |
| 11.67 | 7.57 | 88.3 |
| 16.06 | 5.51 | 380.0 |
| 17.34 | 5.11 | 9972 |
| 18.27 | 4.85 | 464.7 |
| 18.69 | 4.74 | 1532 |
| 19.49 | 4.55 | 387.5 |
| 20.66 | 4.30 | 587.9 |
| 21.09 | 4.21 | 3023 |
| 21.70 | 4.09 | 1257 |
| 22.10 | 4.02 | 5053 |
| 22.76 | 3.90 | 636.7 |
| 23.46 | 3.79 | 723.6 |
| 23.74 | 3.74 | 238.5 |
| 25.06 | 3.55 | 6064 |
| 25.70 | 3.46 | 7656 |
| 26.12 | 3.41 | 301.6 |
| 26.32 | 3.38 | 352.7 |
| 27.64 | 3.23 | 147.8 |
| 27.78 | 3.21 | 140.2 |
| 28.31 | 3.15 | 2450 |
| 28.49 | 3.13 | 300.2 |
| 29.04 | 3.07 | 293.7 |
| 29.95 | 2.98 | 1525 |
| 31.59 | 2.83 | 197.4 |
| 31.82 | 2.81 | 125.7 |
| 32.25 | 2.77 | 707.3 |
| 33.22 | 2.69 | 71.4 |
| 34.21 | 2.62 | 391.5 |
| 34.42 | 2.60 | 415.4 |
| 35.08 | 2.56 | 291.9 |
| 35.53 | 2.52 | 173.3 |
| 36.33 | 2.47 | 100.8 |
| 36.70 | 2.45 | 316.9 |
| 37.16 | 2.42 | 309.8 |
| 37.65 | 2.39 | 53.2 |
| 39.02 | 2.31 | 22.1 |
| 39.60 | 2.27 | 156.6 |
| 39.81 | 2.26 | 392.5 |

The XRPD pattern of Compound 1 Form A bulk powder is shown in FIG. 25. Comparison of observed XRPD pattern of Compound 1 Form A bulk powder corresponds well with the calculated XRPD pattern (FIG. 24).

TGA of Compound 1 Form A is shown in FIG. 26. A weight loss of 0.6% was observed up to 150° C.

DSC of Compound 1 Form A is shown in FIG. 27. One endotherm was observed at 116.42° C.

A comparison of Compound 1 Lot I is shown in FIG. 28 and verifies that Compound 1 Lot I matches Form A.

### Example 8. Preparation of a Compound 1 Formulation

Polyethylene glycol 300 (175 kg) and polysorbate 80 (35 kg) were combined in a 440 L vessel. Compound 1 (3.5 kg, adjusted for purity, water content, and residual solvent) was added and stirred for 60 minutes. Phosphate buffered saline was filtered and added to the solution until the total mass of the compounding mixture was 381.5 kg. 1.0 N HCl and/or 1.0 N NaOH was added to bring pH to 7.5-7.9. The resulting solution was filtered through 0.45 μm and 0.22 μm polish filters in succession, then filled into 20 mL Type 1 glass vials with a target fill weight of 25.12 g per vial and nitrogen over-fill. Vials were capped with B2-40 West stoppers and sealed.

### Example 9. Compound 1 Treatment Attenuates Ischemia-Reperfusion-Induced Renal Injury in Rat and Dog Models

The effect of Compound 1 on reducing ischemia-reperfusion-induced renal injury and mortality in rats and dogs subjected to normothermic renal ischemia and reperfusion (nRIR) was evaluated.

In a first study, 32 male and 20 female Sprague Dawley (SD) rats were subjected to 60-min renal ischemia and 24-hr reperfusion. Compound 1 (2 mg/kg, intravenous [IV]; N=26) or vehicle (N=26) was given pre-ischemia and 18 hours post-reperfusion. At the onset of reperfusion, the contralateral (right) kidney was excised. Blood urea nitrogen (BUN) and creatinine (Cr) levels were assessed at 24 h pre-sacrifice.

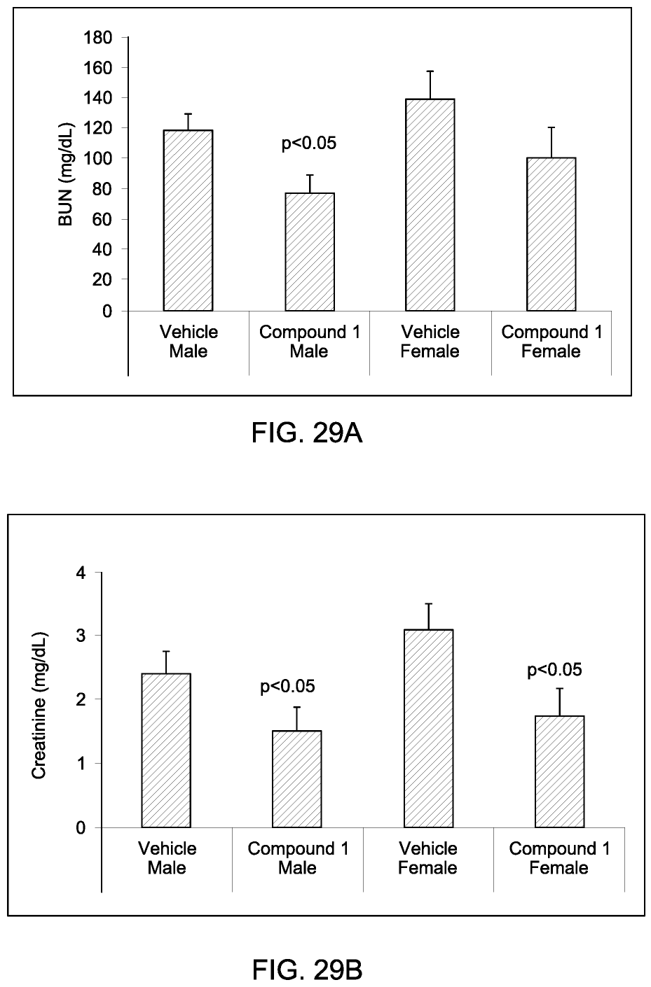

Compound 1 treatment immediately prior to ischemic injury and 18 hours post-reperfusion significantly reduced BUN levels in male rats (FIG. 29A) and SCr levels in both male and female rats (FIG. 29B) subjected to nRIR.

In a second study, 136 SD rats were subjected to 60-min renal ischemia and 96-hr reperfusion. Animals were dosed with Compound 1 IV (0.2, N=48; or 2 mg/kg, N=15) or vehicle (N=70) at 24-hr post-onset of reperfusion, then once daily (QD) for the next four days. Blood and urine were collected daily (24, 48, 72, and 96 hours); mortality was recorded. Normal rats (n=3) not subjected to ischemia and reperfusion, and not treated with Compound 1 nor vehicle, were included as additional controls.

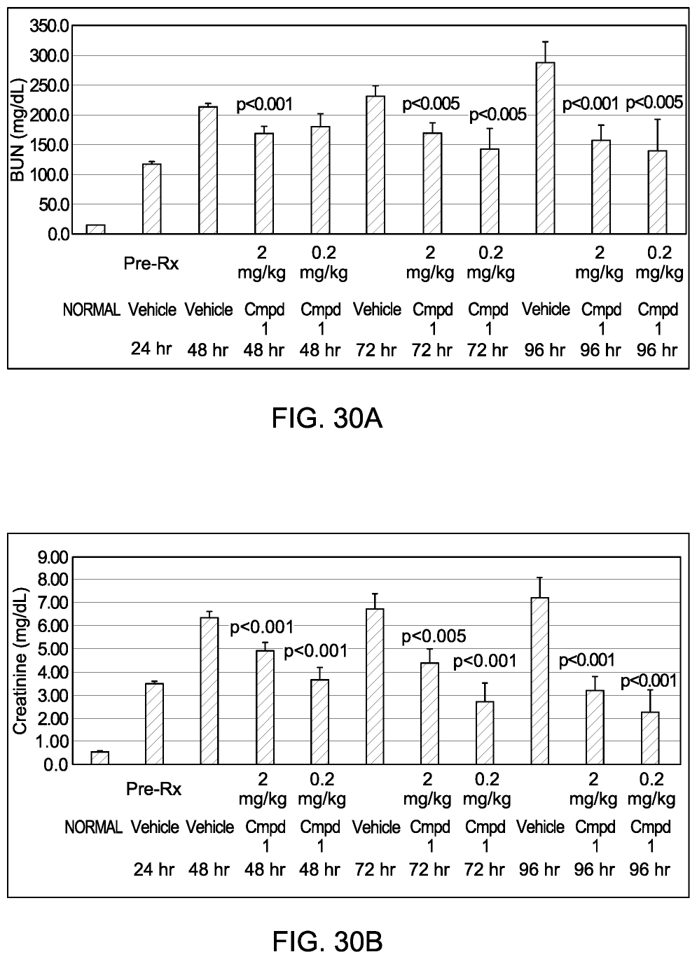

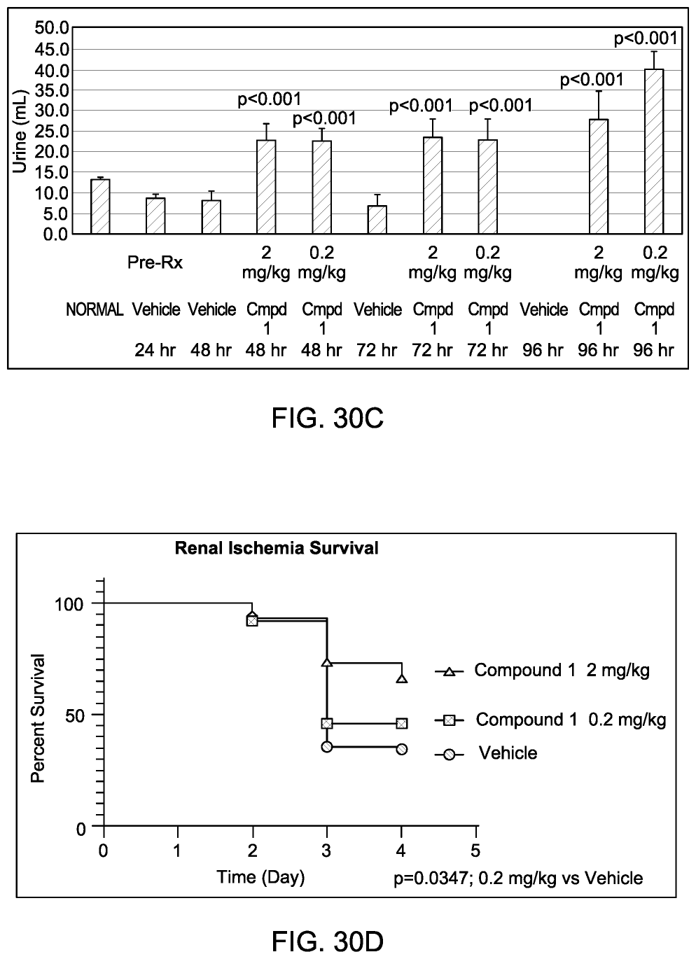

Once daily treatment with Compound 1 for four days, starting 24 hours post-ischemic injury significantly reduced BUN (FIG. 30A) and SCr (FIG. 30B) levels, increased urine output (FIG. 30C), and improved survival (FIG. 30D) in male rats subjected to nRIR.

In a third study, 13 beagle dogs were subjected to 120-min left kidney renal ischemia and 7 days of reperfusion. At the onset of reperfusion, the contralateral kidney was excised. Dogs were randomized to one of the following groups: (1) vehicle (IV, QD, n=4); (2) Compound 1 (10 mg/kg, IV, QD, n=4), both started at the onset of reperfusion (Day 1); or (3) Compound 1 delayed treatment (10 mg/kg, IV, QD, n=5) started one day post ischemia-reperfusion (Day 2). Dogs were dosed QD until and including Day 4. Blood was collected every 24 hours from an indwelling catheter for a total of 8 days that included a day prior to ischemia-reperfusion (Day 0), and SCr and BUN were measured.

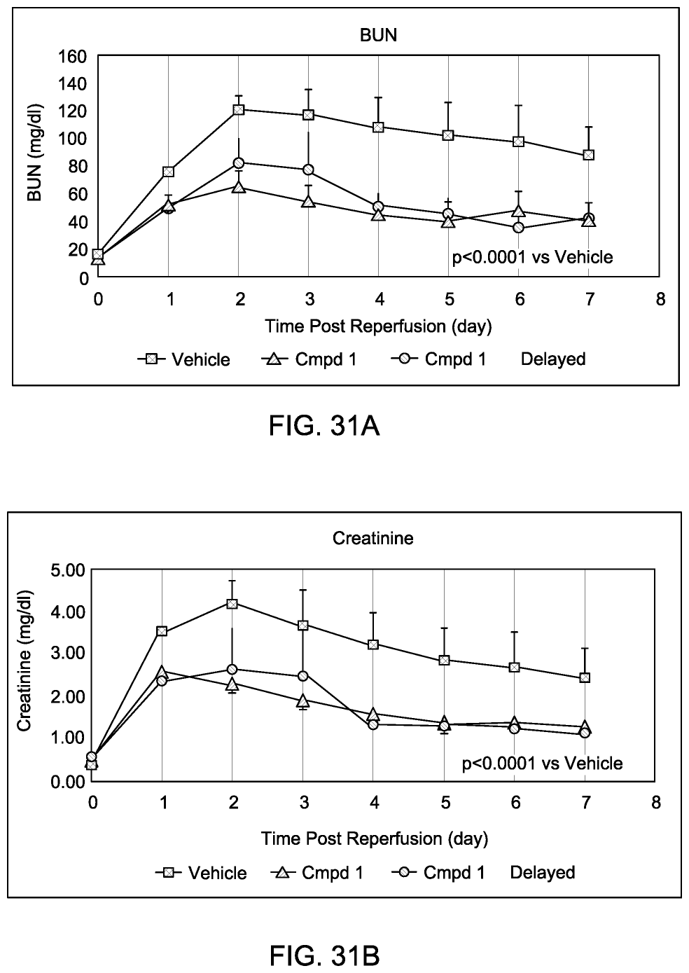

Initiating once daily treatment of Compound 1 at the onset of reperfusion or one day post-reperfusion significantly improved BUN (FIG. 31A) and SCr (FIG. 31B) levels in dogs subjected to nRIR.

Overall, in nRIR animal models, immediate or delayed treatment with Compound 1 resulted in a statistically significant improvement in (1) markers of renal function (SCr and BUN levels) in SD rats and Beagle dogs; and (2) urine output and survival in SD rats. These results provide evidence that Compound 1 can improve kidney function after ischemia reperfusion injury.

### Example 10. Potential Cost-Savings Associated with Compound 1 for the Treatment of DGF in Kidney Transplantation

A medical cost offset model based on results of a Phase 2, double-blind, randomized controlled trial of Compound 1 (e.g., the trial described in Example 1) from the perspective of a US integrated disease networks with a one-year time horizon was used to evaluate potential cost-savings associated with Compound 1 therapy.

The model assessed costs across three time horizons calculated from several data sources. Transplant hospital days, graft failure rates, and eGFRs were collected from the Phase 2 trial. Graft failure in the Compound 1 arm was inflated from an observed 0.0% to 5.58% to reflect epidemiologic norms for non-DGF patients (Butala, N. M., et al. Transplantation. 2013; 95(8):1008-1014). Months 2-12 hospitalizations and costs were based on results from peer-reviewed literature and applied to the phase 2 data (Butala 2013; USRDS Annual Data Report 2018; USRDS Annual Data Report 2013; Kim D W, et al., Clin Transplant. 2020 Jun. 23:e14022. doi: 10.1111/ctr.14022; Keong, F. M., et al., Kidney Int. Reports. 2016; 1(4):269-278). Costs were generated via a Premier Hospital Database analysis and the 2018 United States Renal Data System (USRDS) annual data report. Costs for 2017 were inflated to 2018 dollars and adjusted by 58% to reflect Medicare costs (from USRDS) vs. IDN per USRDS Annual Data Report 2013. The cost of Compound 1 was unknown and therefore was not included in this analysis.

The results of the analysis are summarized in Table 11.

TABLE 11

| | Placebo | Compound 1 |
|---|---|---|
| Model Inputs and Data Sources | Arm | Arm |
| MONTH 1 | | |
| Transplant hospitalization length of stay, mean, days (Phase | 11.4 | 7.6 |
| 2 trial results) | | |
| Transplant hospitalization cost, mean (Kim 2020) | $118,534 | $106,296 |
| MONTHS 2-6 | | |
| 6-month eGFR, mean, mL/min/1.73 m² (Phase 2 trial | 39.5 | 47.5 |
| results) | | |
| Readmissions, mean (Keong 2016) | 40% | 30% |
| Readmission costs, mean (Kim 2020) | $7,748 | $7,748 |
| MONTHS 7-12 | | |
| Graft Failures | | |
| Graft failure rate at 1 year (Phase 2 trial results) | 1/9 (22.2%) | 0/19 (0.0%) |
| Imputed graft failure rate, Compound 1 (Butala 2013) | — | 5.58% |
| Graft failure cost, mean (USRDS, 2013 & 2018) | $162,482 | $162,482 |
| Non-Graft Failures | | |
| Non-graft failure rate at 1 year (Phase 2 trial results) | 8/9 (77.8%) | 19/19 (100%) |
| Imputed non-graft failure rate, Compound 1 (Butala 2013) | — | 94.4% |
| Rehospitalizations, mean (Keong 2016)* | 0.18 | 0.16 |
| Rehospitalization cost, mean (Kim 2020) | $7,748 | $7,748 |
| Results | | |
| Initial transplant hospitalization | $118,535 | $106,296 |
| Readmissions, months 2-6 | $3,099 | $2,324 |
| Graft failure costs, months 7-12 | $32,972 | $6,739 |
| Non-graft failure costs, months 7-12 | $1,059 | $1,144 |
| TOTAL COSTS | $155,665 | $116,504 |

*Rehospitalization rates from 7-18 months were adjusted to reflect 7-12 months to fit the model time horizon.

Initial hospitalization (A $12,239), readmissions (A $775), and graft failure costs (A $26,233) were higher in the placebo arm of the model. Non-graft failure 7-12 month hospitalization costs were slightly higher in the Compound 1 arm (A $85), due to the fact that fewer patients experienced graft failure. The total calculated, per-patient, cost offset associated with Compound 1 in the first year after transplantation was $39,162.

On an annual basis, treatment with Compound 1 may prevent graft failures and lower total costs. Limitations of the model include a conservative under-estimation of the clinical impact of Compound 1, a one-year time horizon, and no explicit consideration for treatment-emergent adverse events or health-related quality of life. Additionally, from months 0-6, only hospitalization costs were included in this model. Outpatient dialysis and pharmacy costs were not included for either patient group. Therefore, this analysis looks only at cost offset due to hospitalizations and graft failure costs. Yet, even so, potential cost-savings of $39,162 demonstrate economic benefits of Compound 1 therapy in these patients.

1. A method comprising administering a composition providing (E)-3-[2-(2-thienyl)vinyl]-1H-pyrazole to a subject or population of subjects who have undergone renal transplantation.

2. A method of improving kidney graft function comprising administering a composition providing (E)-3-[2-(2-thienyl)vinyl]-1H-pyrazole to a subject or population of subjects who have undergone renal transplantation.

3. A method of treating delayed graft function comprising administering a composition providing (E)-3-[2-(2-thienyl)vinyl]-1H-pyrazole to a subject or population of subjects who have undergone renal transplantation.

4. A method of increasing life expectancy in a subject or population of subjects who have undergone renal transplantation, comprising administering a composition providing (E)-3-[2-(2-thienyl)vinyl]-1H-pyrazole.

5. The method of any one of claims 1-4, wherein the composition is administered intravenously.

6. The method of any one of claims 1-5, wherein the composition is administered in a dose of 2 mg/kg.

7. The method of any one of claims 1-6, wherein the composition is administered once daily.

8. The method of any one of claims 1-7, wherein the composition is administered once daily for three days.

9. The method of any one of claims 1-8, wherein the composition is administered within about 30 h after renal transplantation.

10. The method of any one of claims 1-9, wherein the composition comprises:
about 6 mg/mL (E)-3-[2-(2-thienyl)vinyl]-1H-pyrazole;
about 20% (w/v) to about 40% (w/v) polyethylene glycol 300;
about 5% (w/v) to about 15% (w/v) polysorbate 80; and
one or more aqueous components selected from phosphate buffered saline and normal saline.

11. The method of any one of claims 1-10, wherein the subject is at risk of delayed graft function.

12. The method of claim 11, wherein the subject has an average urine output of less than about 50 mL/hour over any consecutive 8 hours in the first 24 hours after transplantation.

13. The method of any one of claims 1-12, wherein the subject has received a kidney from a deceased donor.

14. The method of any one of claims 1-13, wherein the composition is administered to a population of subjects according to a regimen established to achieve one or more of:
(i) a greater mean estimated glomerular filtration rate at about 6 months or about 12 months after renal transplantation;
(ii) a greater mean increase in estimated glomerular filtration rate from baseline at about 6 months or about 12 months after renal transplantation;
(iii) a lesser mean serum creatinine concentration at about 6 months or about 12 months after renal transplantation; and
(iv) a greater mean decrease in serum creatinine concentration from baseline at about 6 months or about 12 months after renal transplantation,
relative to a comparable reference population.

15. The method of claim 14, wherein the reference population has received an otherwise comparable reference composition that does not provide (E)-3-[2-(2-thienyl)vinyl]-1H-pyrazole.

16. The method of claim 14 or 15, wherein the regimen has been established to achieve a greater mean estimated glomerular filtration rate at about 12 months after renal transplantation, relative to a comparable reference population.

17. The method of any one of claims 14-16, wherein the estimated glomerular filtration rate was calculated using the CKD-EPI equation.

18. The method of any one of claims 14-17, wherein the regimen has been established to achieve a mean estimated glomerular filtration rate of from about 45 mL/min/1.73 m² to about 55 mL/min/1.73 m² at about 12 months after renal transplantation.

19. The method of any one of claims 14-18, wherein the regimen has been established to achieve a greater mean increase in estimated glomerular filtration rate from baseline at about 12 months after renal transplantation.

20. The method of any one of claims 14-19, wherein the regimen has been established to achieve a mean increase in estimated glomerular filtration rate from baseline of from about from about 36 mL/min/1.73 m² to about 45 mL/min/1.73 m² at about 12 months after renal transplantation.

21. The method of any one of claims 14-20, wherein the regimen has been established to achieve a lesser mean serum creatinine concentration at about 12 months after renal transplantation, relative to a comparable reference population.

22. The method of any one of claims 14-21, wherein the regimen has been established to achieve a mean serum creatinine concentration of from about 1.45 mg/dL to about 1.75 mg/dL at about 12 months after renal transplantation.

23. The method of any one of claims 14-22, wherein the regimen has been established to achieve a greater mean decrease in serum creatinine concentration from baseline at about 12 months after renal transplantation.

24. The method of any one of claims 14-23, wherein the regimen has been established to achieve a mean decrease in serum creatinine concentration from baseline of from about from about 6.1 mg/dL to about 6.5 mg/dL at about 12 months after renal transplantation.

25. The method of any one of claims 14-24, wherein the regimen has further been established to achieve one or more of:
(v) a greater incidence of achieving 1200 cc urine output over a 24 hour period within 28 days after renal transplantation;
(vi) a shorter median time to achieve 1200 cc urine output over a 24 hour period within 28 days after renal transplantation;
(vii) a lesser mean number of dialysis sessions per subject within 28 days after renal transplantation;
(viii) a shorter mean duration of dialysis within 28 days after renal transplantation;
(ix) a shorter mean length of hospitalization after renal transplantation;
(x) a lower incidence of graft failure within 12 months after renal transplantation;
(xi) a lesser mean serum creatinine concentration at about 7, 14, or 28 days after renal transplantation;
(xii) a greater mean decrease in serum creatinine concentration from baseline at about 7 days, about 14 days, or about 28 days after renal transplantation;
(xiii) a greater mean estimated glomerular filtration rate at about 7, 14, or 28 days after renal transplantation; and
(xiv) a greater mean increase in estimated glomerular filtration rate from baseline at about 7 days, about 14 days, or about 28 days after renal transplantation,
relative to a comparable reference population.

26. The method of any one of claims 14-25, wherein the regimen has further been established to achieve one or more of:
(xv) a greater proportion of patients with an estimated glomerular filtration rate greater than about 30 mL/min/1.73 m² at about 30 days, about 90 days, about 6 months or about 12 months after renal transplantation;
(xvi) a lesser proportion of patients with (1) PNF (defined as a continuous requirement for dialysis for at least 60 days after renal transplantation); (2) DGF (assessed by proportion of patients requiring dialysis within 7 days after renal transplantation); or (3) SGF (defined as having a SCr<3 mg/dL within the first 7 days after renal transplantation without dialysis required);
(xvii) a shorter mean length of hospitalization after renal transplantation; and
(xviii) a lesser number of days a patient remains dialysis dependent within the first 30 days after renal transplantation,
relative to a comparable reference population.

27. The method of any one of claims 1-26, wherein the subject or population of subjects is suffering from an active malignancy or has suffered from a solid, metastatic or hematologic malignancy.

28. The method of any one of claims 1-27, wherein the subject or population of subjects has not been assessed for an active malignancy or a history of a solid, metastatic or hematologic malignancy.

29. A method comprising:
administering a composition providing (E)-3-[2-(2-thienyl)vinyl]-1H-pyrazole to a subject in need thereof,
wherein the subject is suffering from an active malignancy or has suffered from a solid, metastatic or hematologic malignancy.

30. The method of claim 29, wherein the subject has not suffered from a basal or squamous cell carcinoma of the skin that has been treated and/or removed.

31. The method of claim 29 or 30, wherein the subject has suffered from a solid, metastatic or hematologic malignancy within 5 years prior to administration of the composition providing (E)-3-[2-(2-thienyl)vinyl]-1H-pyrazole.

32. A method comprising:
administering a composition providing (E)-3-[2-(2-thienyl)vinyl]-1H-pyrazole to a subject in need thereof,
wherein the subject has not been assessed for an active malignancy or a history of a solid, metastatic or hematologic malignancy.

33. The method of any one of claims 29-32, wherein the composition is administered intravenously.

34. The method of any one of claims 29-33, wherein the composition is administered in a dose of about 2 mg/kg.

35. The method of any one of claims 29-34, wherein the composition is administered once daily.

36. The method of any one of claims 29-35, wherein the composition is administered once daily for three or four days.

37. The method of any one of claims 29-36, wherein the composition comprises:
about 6 mg/mL (E)-3-[2-(2-thienyl)vinyl]-1H-pyrazole;
about 20% (w/v) to about 40% (w/v) polyethylene glycol 300;
about 5% (w/v) to about 15% (w/v) polysorbate 80; and
one or more aqueous components selected from phosphate buffered saline and normal saline.
